SEQUENCE LISTING (218 sequences)

SEQ ID NO: 1 - protein, 5 aa, synthetic construct
  REGION 1..5: note = CDR sequence
SYSMS

SEQ ID NO: 2 - protein, 17 aa, synthetic construct
  REGION 1..17: note = CDR sequence
HISSGGSSTYYPDTVKG

SEQ ID NO: 3 - protein, 13 aa, synthetic construct
  REGION 1..13: note = CDR sequence
QFYYGSSFWYFDV

SEQ ID NO: 4 - protein, 15 aa, synthetic construct
  REGION 1..15: note = CDR sequence
KASQSVDFDGDSYMN

SEQ ID NO: 5 - protein, 7 aa, synthetic construct
  REGION 1..7: note = CDR sequence
AASNLES

SEQ ID NO: 6 - protein, 9 aa, synthetic construct
  REGION 1..9: note = CDR sequence
QQSNEDPPT

SEQ ID NO: 7 - protein, 5 aa, synthetic construct
  REGION 1..5: note = CDR sequence
SYWMH

SEQ ID NO: 8 - protein, 17 aa, synthetic construct
  REGION 1..17: note = CDR sequence
YINPSTGYTEYNQKFKD

SEQ ID NO: 9 - protein, 8 aa, synthetic construct
  REGION 1..8: note = CDR sequence
GVRYYFDY

SEQ ID NO: 10 - protein, 17 aa, synthetic construct
  REGION 1..17: note = CDR sequence
KSSQSLLNSRTRKNYLA

SEQ ID NO: 11 - protein, 7 aa, synthetic construct
  REGION 1..7: note = CDR sequence
WASTRES

SEQ ID NO: 12 - protein, 8 aa, synthetic construct
  REGION 1..8: note = CDR sequence
KQSYNLLT

SEQ ID NO: 13 - protein, 5 aa, synthetic construct
  REGION 1..5: note = CDR sequence
SYWMH

SEQ ID NO: 14 - protein, 17 aa, synthetic construct
  REGION 1..17: note = CDR sequence
YINPNTGYTEYNQKFKD

SEQ ID NO: 15 - protein, 8 aa, synthetic construct
  REGION 1..8: note = CDR sequence
GTAYYFDY

SEQ ID NO: 16 - protein, 17 aa, synthetic construct
  REGION 1..17: note = CDR sequence
KSSQSLLNSRTRKNYLA

SEQ ID NO: 17 - protein, 7 aa, synthetic construct
  REGION 1..7: note = CDR sequence
WASTRES

SEQ ID NO: 18 - protein, 8 aa, synthetic construct
  REGION 1..8: note = CDR sequence
KQSYNLQT

SEQ ID NO: 19 - protein, 10 aa, synthetic construct
  REGION 1..10: note = CDR sequence
GFTFSSYSMS

SEQ ID NO: 20 - protein, 6 aa, synthetic construct
  REGION 1..6: note = CDR sequence
SSGGSS

SEQ ID NO: 21 - protein, 13 aa, synthetic construct
  REGION 1..13: note = CDR sequence
QFYYGSSFWYFDV

SEQ ID NO: 22 - protein, 15 aa, synthetic construct
  REGION 1..15: note = CDR sequence
KASQSVDFDGDSYMN

SEQ ID NO: 23 - protein, 7 aa, synthetic construct
  REGION 1..7: note = CDR sequence
AASNLES

SEQ ID NO: 24 - protein, 9 aa, synthetic construct
  REGION 1..9: note = CDR sequence
QQSNEDPPT

SEQ ID NO: 25 - protein, 10 aa, synthetic construct
  REGION 1..10: note = CDR sequence
GYTFTSYWMH

SEQ ID NO: 26 - protein, 6 aa, synthetic construct
  REGION 1..6: note = CDR sequence
NPSTGY

SEQ ID NO: 27 - protein, 8 aa, synthetic construct
  REGION 1..8: note = CDR sequence
GVRYYFDY

SEQ ID NO: 28 - protein, 17 aa, synthetic construct
  REGION 1..17: note = CDR sequence
KSSQSLLNSRTRKNYLA

SEQ ID NO: 29 - protein, 7 aa, synthetic construct
  REGION 1..7: note = CDR sequence
WASTRES

SEQ ID NO: 30 - protein, 8 aa, synthetic construct
  REGION 1..8: note = CDR sequence
KQSYNLLT

SEQ ID NO: 31 - protein, 10 aa, synthetic construct
  REGION 1..10: note = CDR sequence
GYTLTSYWMH

SEQ ID NO: 32 - protein, 6 aa, synthetic construct
  REGION 1..6: note = CDR sequence
NPNTGY

SEQ ID NO: 33 - protein, 8 aa, synthetic construct
  REGION 1..8: note = CDR sequence
GTAYYFDY

SEQ ID NO: 34 - protein, 17 aa, synthetic construct
  REGION 1..17: note = CDR sequence
KSSQSLLNSRTRKNYLA

SEQ ID NO: 35 - protein, 7 aa, synthetic construct
  REGION 1..7: note = CDR sequence
WASTRES

SEQ ID NO: 36 - protein, 8 aa, synthetic construct
  REGION 1..8: note = CDR sequence
KQSYNLQT

SEQ ID NO: 37 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 38 - protein, 288 aa, Mus musculus
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 39 - protein, 288 aa, synthetic construct
  REGION 1..288: note = Chimeric PD-1
MWVRQVPWSFTWAVLQLSWQSGWLLEVPNGPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 40 - protein, 122 aa, synthetic construct
  REGION 1..122: note = heavy chain variable domain
EVQLLESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGQGTTVTVSS

SEQ ID NO: 41 - protein, 122 aa, synthetic construct
  REGION 1..122: note = heavy chain variable domain
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSS

SEQ ID NO: 42 - protein, 122 aa, synthetic construct
  REGION 1..122: note = heavy chain variable domain
EVKLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSS

SEQ ID NO: 43 - protein, 111 aa, synthetic construct
  REGION 1..111: note = light chain variable domain
DNVLTQSPATLSLSPGERATLSCKASQSVDFDGDSYMNWYQQKPGQPPRLLIYAASNLESGIPARFSGSGPGTDFTLTISSLEPEDFATYYCQQSNEDPPTFGGGTKVEIK

SEQ ID NO: 44 - protein, 111 aa, synthetic construct
  REGION 1..111: note = light chain variable domain
DNVLTQSPATLSVSPGERATLSCKASQSVDFDGDSYMNWYQQKPGQPPRLLIYAASNLESGIPARFSGSGPGTDFTLTISSLEPEDFATYYCQQSNEDPPTFGGGTKVEIK

SEQ ID NO: 45 - protein, 111 aa, synthetic construct
  REGION 1..111: note = light chain variable domain
DNVLTQSPATLSVSPGERATLSCKASQSVDFDGDSYMNWYQQKPGQPPRLLIYAASNLESGIPARFSGSGSGTDFTLTISSVEPEDFATYYCQQSNEDPPTFGGGTKVEIK

SEQ ID NO: 46 - protein, 117 aa, synthetic construct
  REGION 1..117: note = heavy chain variable domain
QVQLQQSGAEVKKPGASVKMSCKASGYTFTSYWMHWVRQAPGQGLEWIGYINPSTGYTEYNQKFKDRATLTADTSTSTAYMELSSLRSEDTAVYYCARGVRYYFDYWGQGTLVTVSS

SEQ ID NO: 47 - protein, 117 aa, synthetic construct
  REGION 1..117: note = heavy chain variable domain
QVQLQQSGAEVKKPGASVKMSCKASGYTFTSYWMHWVKQRPGQGLEWIGYINPSTGYTEYNQKFKDRATLTADKSTSTAYMELSSLRSEDTAVYYCARGVRYYFDYWGQGTLVTVSS

SEQ ID NO: 48 - protein, 117 aa, synthetic construct
  REGION 1..117: note = heavy chain variable domain
QVQLQQSGAEVAKPGASVKMSCKASGYTFTSYWMHWVKQRPGQGLEWIGYINPSTGYTEYNQKFKDRATLTADTSTSTAYMELSSLRSEDTAVYYCARGVRYYFDYWGQGTLLTVSS

SEQ ID NO: 49 - protein, 117 aa, synthetic construct
  REGION 1..117: note = heavy chain variable domain
QVQLQQSGAEVAKPGASVKMSCKASGYTFTSYWMHWVKQRPGQGLEWIGYINPSTGYTEYNQKFKDRATLTADKSTSTAYMELSSLTSEDTAVYYCARGVRYYFDYWGQGTLLTVSS

SEQ ID NO: 50 - protein, 112 aa, synthetic construct
  REGION 1..112: note = light chain variable domain
DIVMTQSPDSLAVSLGERATISCKSSQSLLNSRTRKNYLAWYQQKPGQSPKLLIYWASTRESGVPDRFSGSGSGTDFTLTISSVQAEDVAVYYCKQSYNLLTFGQGTKLELK

SEQ ID NO: 51 - protein, 112 aa, synthetic construct
  REGION 1..112: note = light chain variable domain
DIVMTQSPDSLAVSLGERATMSCKSSQSLLNSRTRKNYLAWYQQKPGQSPKLLIYWASTRESGVPDRFSGSGSGTDFTLTISSVQAEDVAVYYCKQSYNLLTFGQGTKLELK

SEQ ID NO: 52 - protein, 112 aa, synthetic construct
  REGION 1..112: note = light chain variable domain
DIVMTQSPSSLAVSAGERVTMSCKSSQSLLNSRTRKNYLAWYQQKPGQSPKLLIYWASTRESGVPDRFSGSGSGTDFTLTISSVQAEDVAVYYCKQSYNLLTFGQGTKLELK

SEQ ID NO: 53 - protein, 122 aa, synthetic construct
  REGION 1..122: note = heavy chain variable domain
EVKLVESGGGLVQPGGSLKLSCAASGFTFSSYSMSWVRQTPEKRLEWVAHISSGGSSTYYPDTVKGRFTISRDNARNTLYLQMSSLKSEDTAMYYCTRQFYYGSSFWYFDVWGAGTTVTVSS

SEQ ID NO: 54 - protein, 111 aa, synthetic construct
  REGION 1..111: note = light chain variable domain
DNVLTQSPVSLAVSLGQRATISCKASQSVDFDGDSYMNWYQQKPGQPPKLLIYAASNLESGIPARFSGSGSGTDFTLNIHPVEEEDAATYYCQQSNEDPPTFGGGTKLEIK

SEQ ID NO: 55 - protein, 117 aa, synthetic construct
  REGION 1..117: note = heavy chain variable domain
QVQLQQSGAELAKPGASVKMSCKASGYTFTSYWMHWVKQRPGQGLEWIGYINPSTGYTEYNQKFKDKATLTADKSSSTAYMQLSSLTSEDSAVYYCARGVRYYFDYWGQGTTLTVSS

SEQ ID NO: 56 - protein, 112 aa, synthetic construct
  REGION 1..112: note = light chain variable domain
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLLTFGAGTKLELK

SEQ ID NO: 57 - protein, 117 aa, synthetic construct
  REGION 1..117: note = heavy chain variable domain
QVQLQQSGAELAKPGASLKMSCKASGYTLTSYWMHWVKQRPGQGLEWIGYINPNTGYTEYNQKFKDKATLTADKSSSTAYMQLSSLTSEDSAVYYCARGTAYYFDYWGQGTTLTVSS

SEQ ID NO: 58 - protein, 112 aa, synthetic construct
  REGION 1..112: note = light chain variable domain
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLQTFGGGTKLEIK

SEQ ID NO: 59 - protein, 5 aa, synthetic construct
  REGION 1..5: note = CDR sequence
DYYMY

SEQ ID NO: 60 - protein, 17 aa, synthetic construct
  REGION 1..17: note = CDR sequence
YISYGGDSTFYPDTVRG

SEQ ID NO: 61 - protein, 12 aa, synthetic construct
  REGION 1..12: note = CDR sequence
PAPSAHSYYLDY

SEQ ID NO: 62 - protein, 11 aa, synthetic construct
  REGION 1..11: note = CDR sequence
RASQDISNYLN

SEQ ID NO: 63 - protein, 8 aa, synthetic construct
  REGION 1..8: note = CDR sequence
YYTSRLHS

SEQ ID NO: 64 - protein, 9 aa, synthetic construct
  REGION 1..9: note = CDR sequence
QQGKTLPFT

SEQ ID NO: 65 - protein, 5 aa, synthetic construct
  REGION 1..5: note = CDR sequence
SYYIY

SEQ ID NO: 66 - protein, 17 aa, synthetic construct
  REGION 1..17: note = CDR sequence
GINPRNGGTNFNEKFKS

SEQ ID NO: 67 - protein, 6 aa, synthetic construct
  REGION 1..6: note = CDR sequence
HGNGVY

SEQ ID NO: 68 - protein, 16 aa, synthetic construct
  REGION 1..16: note = CDR sequence
RSSQSLLHSNGNTYLH

SEQ ID NO: 69 - protein, 7 aa, synthetic construct
  REGION 1..7: note = CDR sequence
QVSNRFS

SEQ ID NO: 70 - protein, 9 aa, synthetic construct
  REGION 1..9: note = CDR sequence
SQTTHVPWT

SEQ ID NO: 71 - protein, 5 aa, synthetic construct
  REGION 1..5: note = CDR sequence
SGYWN

SEQ ID NO: 72 - protein, 16 aa, synthetic construct
  REGION 1..16: note = CDR sequence
FISYSGSTYYTPSLKS

SEQ ID NO: 73 - protein, 10 aa, synthetic construct
  REGION 1..10: note = CDR sequence
FRRYDDGVDY

SEQ ID NO: 74 - protein, 11 aa, synthetic construct
  REGION 1..11: note = CDR sequence
RASHEISGYLS

SEQ ID NO: 75 - protein, 7 aa, synthetic construct
  REGION 1..7: note = CDR sequence
AASTLAS

SEQ ID NO: 76 - protein, 9 aa, synthetic construct
  REGION 1..9: note = CDR sequence
LQYSSYPWT

SEQ ID NO: 77 - protein, 10 aa, synthetic construct
  REGION 1..10: note = CDR sequence
GFTFSDYYMY

SEQ ID NO: 78 - protein, 6 aa, synthetic construct
  REGION 1..6: note = CDR sequence
SYGGDS

SEQ ID NO: 79 - protein, 12 aa, synthetic construct
  REGION 1..12: note = CDR sequence
PAPSAHSYYLDY

SEQ ID NO: 80 - protein, 11 aa, synthetic construct
  REGION 1..11: note = CDR sequence
RASQDISNYLN

SEQ ID NO: 81 - protein, 7 aa, synthetic construct
  REGION 1..7: note = CDR sequence
YTSRLHS

SEQ ID NO: 82 - protein, 9 aa, synthetic construct
  REGION 1..9: note = CDR sequence
QQGKTLPFT

SEQ ID NO: 83 - protein, 10 aa, synthetic construct
  REGION 1..10: note = CDR sequence
GYTFISYYIY

SEQ ID NO: 84 - protein, 6 aa, synthetic construct
  REGION 1..6: note = CDR sequence
NPRNGG

SEQ ID NO: 85 - protein, 6 aa, synthetic construct
  REGION 1..6: note = CDR sequence
HGNGVY

SEQ ID NO: 86 - protein, 16 aa, synthetic construct
  REGION 1..16: note = CDR sequence
RSSQSLLHSNGNTYLH

SEQ ID NO: 87 - protein, 7 aa, synthetic construct
  REGION 1..7: note = CDR sequence
QVSNRFS

SEQ ID NO: 88 - protein, 9 aa, synthetic construct
  REGION 1..9: note = CDR sequence
SQTTHVPWT

SEQ ID NO: 89 - protein, 10 aa, synthetic construct
  REGION 1..10: note = CDR sequence
GDSVSSGYWN

SEQ ID NO: 90 - protein, 5 aa, synthetic construct
  REGION 1..5: note = CDR sequence
SYSGS

SEQ ID NO: 91 - protein, 10 aa, synthetic construct
  REGION 1..10: note = CDR sequence
FRRYDDGVDY

SEQ ID NO: 92 - protein, 11 aa, synthetic construct
  REGION 1..11: note = CDR sequence
RASHEISGYLS

SEQ ID NO: 93 - protein, 7 aa, synthetic construct
  REGION 1..7: note = CDR sequence
AASTLAS

SEQ ID NO: 94 - protein, 9 aa, synthetic construct
  REGION 1..9: note = CDR sequence
LQYSSYPWT

SEQ ID NO: 95 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 96 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 97 - protein, 289 aa, synthetic construct
  REGION 1..289: note = Chimeric CD40
MVSLPRLCALWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 98 - protein, 121 aa, synthetic construct
  REGION 1..121: note = heavy chain variable domain
EVQLVESGGGLVQPGGSLRLSCATSGFTFSDYYMYWVRQAPGKRLEWVAYISYGGDSTFYPDTVRGRFTISRDNAKNSLYLQMNSLRAEDTAVYYCARPAPSAHSYYLDYWGQGTLLTVSS

SEQ ID NO: 99 - protein, 121 aa, synthetic construct
  REGION 1..121: note = heavy chain variable domain
EVQLVESGGGLVQPGGSLRLSCATSGFTFSDYYMYWVRQAPGKRLEWVAYISYGGDSTFYPDTVRGRFTISRDNAKNSLYLQMSSLKAEDTAVYYCARPAPSAHSYYLDYWGQGTLLTVSS

SEQ ID NO: 100 - protein, 121 aa, synthetic construct
  REGION 1..121: note = heavy chain variable domain
EVKLVESGGGLVQPGGSLRLSCATSGFTFSDYYMYWVRQAPEKRLEWVAYISYGGDSTFYPDTVRGRFTISRDNAKNALYLQMSRLKAEDTAVYYCARPAPSAHSYYLDYWGQGTTLTVSS

SEQ ID NO: 101 - protein, 107 aa, synthetic construct
  REGION 1..107: note = light chain variable domain
DIQMTQSPSSLSASVGDRVTITCRASQDISNYLNWYQQKPGGAVKLLIYYTSRLHSGLPSRFSGSGSGTDFTFTISSLQPEDIATYYCQQGKTLPFTFASGTKLEIK

SEQ ID NO: 102 - protein, 107 aa, synthetic construct
  REGION 1..107: note = light chain variable domain
DIQMTQSPSSLSASVGDRVTITCRASQDISNYLNWYQQKPGGAVKLLIYYTSRLHSGLPSRFSGSGSGTDYTFTISSLQPEDIATYYCQQGKTLPFTFASGTKLEIK

SEQ ID NO: 103 - protein, 107 aa, synthetic construct
  REGION 1..107: note = light chain variable domain
DIQMTQSTSSLSASVGDRVTITCRASQDISNYLNWYQQKPGGAVKLLIYYTSRLHSGLPSRFSGSGSGTDYTLTISSLQQEDIATYYCQQGKTLPFTFASGTKLEIK

SEQ ID NO: 104 - protein, 107 aa, synthetic construct
  REGION 1..107: note = light chain variable domain
DIQMTQSTSSLSASVGDRVTITCRASQDISNYLNWYQQKPDGTVKLLIYYTSRLHSGLPSRFSGSGSGTDYTLTISSLQQEDIATYYCQQGKTLPFTFASGTKLEIK

SEQ ID NO: 105 - protein, 115 aa, synthetic construct
  REGION 1..115: note = heavy chain variable domain
QVQLVQSGAEVKKPGASVKVSCKASGYTFISYYIYWVRQAPGQGLEWMGGINPRNGGTNFNEKFKSRVTLTVDTSISTAYMELSRLRSEDTAVYYCARHGNGVYWGQGTTVTVSS

SEQ ID NO: 106 - protein, 115 aa, synthetic construct
  REGION 1..115: note = heavy chain variable domain
QVQLVQSGAEVKKPGASVKVSCKASGYTFISYYIYWVRQAPGQGLEWIGGINPRNGGTNFNEKFKSRATLTVDTSISTAYMELSRLRSEDTAVYYCARHGNGVYWGQGTTVTVSS

SEQ ID NO: 107 - protein, 115 aa, synthetic construct
  REGION 1..115: note = heavy chain variable domain
QVQLVQSGAEVKKPGASVKVSCKASGYTFISYYIYWVRQAPGQGLEWIGGINPRNGGTNFNEKFKSRATLTVDTSISTAYMELSRLRSEDTAVYYCTRHGNGVYWGQGTTVTVSS

SEQ ID NO: 108 - protein, 115 aa, synthetic construct
  REGION 1..115: note = heavy chain variable domain
QVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSS

SEQ ID NO: 109 - protein, 112 aa, synthetic construct
  REGION 1..112: note = light chain variable domain
DVVMTQSPLSLPVTLGQPASISCRSSQSLLHSNGNTYLHWFQQRPGQSPRHLIYQVSNRFSGVPDRFSGSGSGTDFTLKISRVEAEDVGVYYCSQTTHVPWTFGGGTKVEIK

SEQ ID NO: 110 - protein, 112 aa, synthetic construct
  REGION 1..112: note = light chain variable domain
DVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIK

SEQ ID NO: 111 - protein, 112 aa, synthetic construct
  REGION 1..112: note = light chain variable domain
DVVMTQSPLSLPVTLGDPASISCRSSQSLLHSNGNTYLHWYQQRPGQSPNHLIYQVSNRFSGVPDRFSGSGSGTDFTLKISRVEAEDVGVYFCSQTTHVPWTFGGGTKLEIK

SEQ ID NO: 112 - protein, 118 aa, synthetic construct
  REGION 1..118: note = heavy chain variable domain
QVQLQESGPGLVKPSQTLSLTCTVSGDSVSSGYWNWIRKHPGKKLEWIGFISYSGSTYYTPSLKSRVTISRDTSKNQFSLKLSSVTAADTAVYYCARFRRYDDGVDYWGQGTLVTVSS

SEQ ID NO: 113 - protein, 118 aa, synthetic construct
  REGION 1..118: note = heavy chain variable domain
EVQLQESGPGLVKPSQTLSLTCTVSGDSVSSGYWNWIRKHPGKKLEWIGFISYSGSTYYTPSLKSRVTISRDTSKNQFSLKLSSVTAADTAVYYCARFRRYDDGVDYWGQGTLVTVSS

SEQ ID NO: 114 - protein, 118 aa, synthetic construct
  REGION 1..118: note = heavy chain variable domain
EVQLQESGPGLVKPSQTLSLTCTVSGDSVSSGYWNWIRKHPGKKLEYMGFISYSGSTYYTPSLKSRITISRDTSKNQFSLKLSSVTAADTAVYFCARFRRYDDGVDYWGQGTLVTVSS

SEQ ID NO: 115 - protein, 118 aa, synthetic construct
  REGION 1..118: note = heavy chain variable domain
EVQLQESGPGLVKPSQTLSLTCTVSGDSVSSGYWNWIRKFPGKKLEYMGFISYSGSTYYTPSLKSRITISRDTSKNQFSLKLSSVTAADTAVYFCARFRRYDDGVDYWGQGTLVTVSS

SEQ ID NO: 116 - protein, 107 aa, synthetic construct
  REGION 1..107: note = light chain variable domain
DIQMTQSPSAMSASVGDRVTITCRASHEISGYLSWFQQKPGGVIKRLINAASTLASGVPSRFSGSRSGTEFTLTISSLQPEDFATYYCLQYSSYPWTFGGGTKLEIK

SEQ ID NO: 117 - protein, 107 aa, synthetic construct
  REGION 1..107: note = light chain variable domain
DIQMTQSPSAMSASVGDRVTITCRASHEISGYLSWIQQKPGGTIKRLINAASTLASGVPSRFSGSRSGTEFTLTISSLQPEDFADYYCLQYSSYPWTFGGGTKLEIK

SEQ ID NO: 118 - protein, 107 aa, synthetic construct
  REGION 1..107: note = light chain variable domain
DIQMTQSPSAMSASVGDRVTITCRASHEISGYLSWIQQKPGGTIKRLINAASTLASGVPSRFSGSRSGTEYTLTISSLQPEDFADYYCLQYSSYPWTFGGGTKLEIK

SEQ ID NO: 119 - protein, 107 aa, synthetic construct
  REGION 1..107: note = light chain variable domain
DIQMTQSPSAMSASVGDRVTLTCRASHEISGYLSWIQQKPGGTIKRLINAASTLASGVPSRFSGSRSGTEYTLTISSLQPEDFADYYCLQYSSYPWTFGGGTKLEIK

SEQ ID NO: 120 - protein, 121 aa, synthetic construct
  REGION 1..121: note = heavy chain variable domain
EVKLVESGGGLVQPGGSLKLSCATSGFTFSDYYMYWVRQTPEKRLEWVAYISYGGDSTFYPDTVRGRFTISRDNAKNALYLQMSRLKSEDTAVYYCARPAPSAHSYYLDYWGQGTTLTVSS

SEQ ID NO: 121 - protein, 107 aa, synthetic construct
  REGION 1..107: note = light chain variable domain
DIQMTQTTSSLSASLGDRVTISCRASQDISNYLNWYQQKPDGTVKLLIYYTSRLHSGLPSRFSGSGSGTDYSLTISNLEQEDIATYYCQQGKTLPFTFASGTKLEIK

SEQ ID NO: 122 - protein, 115 aa, synthetic construct
  REGION 1..115: note = heavy chain variable domain
QVQLQQPGAELVKPGASVKLSCKASGYTFISYYIYWVKQRPGQGLEWIGGINPRNGGTNFNEKFKSKATLTVDKSSSTAYMQLSSLTSEDSAVYYCTRHGNGVYWGQGTTLTVSS

SEQ ID NO: 123 - protein, 112 aa, synthetic construct
  REGION 1..112: note = light chain variable domain
DVVMTQTPLSLPVSLGDQASISCRSSQSLLHSNGNTYLHWYLQKPGQSPNHLIYQVSNRFSGVPDRFSGSGSGTDFTLKISRVEAEDLGVYFCSQTTHVPWTFGGGTKLEIK

SEQ ID NO: 124 - protein, 118 aa, synthetic construct
  REGION 1..118: note = heavy chain variable domain
EVKLQESGPSLVKPSQTLSLTCSVTGDSVSSGYWNWIRKFPGNKLEYMGFISYSGSTYYTPSLKSRISITRDTSKNQFYLQLNSVTTEDTATYFCARFRRYDDGVDYWGQGTTLTVSS

SEQ ID NO: 125 - protein, 107 aa, synthetic construct
  REGION 1..107: note = light chain variable domain
DIQMTQSPSSLSASLGERVSLTCRASHEISGYLSWIQQKPDGTIKRLINAASTLASGVPKRFSGSRSGSEYSLTINSLESEDFADYYCLQYSSYPWTFGGGTKLEIK

SEQ ID NO: 126 - protein, 115 aa, synthetic construct
  REGION 1..115: note = heavy chain variable domain
QVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 127 - protein, 112 aa, synthetic construct
  REGION 1..112: note = light chain variable domain
DVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIK

SEQ ID NO: 128 - protein, 115 aa, synthetic construct
  REGION 1..115: note = heavy chain variable domain
QVQLQQPGAELVKPGASVKLSCKASGYTFISYYIYWVKQRPGQCLEWIGGINPRNGGTNFNEKFKSKATLTVDKSSSTAYMQLSSLTSEDSAVYYCTRHGNGVYWGQGTTLTVSS

SEQ ID NO: 129 - protein, 112 aa, synthetic construct
  REGION 1..112: note = light chain variable domain
DVVMTQTPLSLPVSLGDQASISCRSSQSLLHSNGNTYLHWYLQKPGQSPNHLIYQVSNRFSGVPDRFSGSGSGTDFTLKISRVEAEDLGVYFCSQTTHVPWTFGCGTKLEIK

SEQ ID NO: 130 - protein, 449 aa, synthetic construct
  REGION 1..449: note = Full-length PD-1 heavy chain of Fab-ScFV-IgG4
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 131 - protein, 218 aa, synthetic construct
  REGION 1..218: note = Full-length PD-1 light chain of Fab-ScFV-IgG4
DNVLTQSPATLSVSPGERATLSCKASQSVDFDGDSYMNWYQQKPGQPPRLLIYAASNLESGIPARFSGSGSGTDFTLTISSVEPEDFATYYCQQSNEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 483 aa, synthetic construct
  REGION 1..483: note = Full-length CD40 arm (version 2) of Fab-ScFV-IgG4
QVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSSASTGGGGSGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 133 - protein, 483 aa, synthetic construct
  REGION 1..483: note = Full-length CD40 arm (version 1) of Fab-ScFV-IgG4
QVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSSASTGGGGSGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIKDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 134 - protein, 710 aa, synthetic construct
  REGION 1..710: note = Full-length modified heavy chain (version 2) of ScFv-HC-IgG4
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 135 - protein, 710 aa, synthetic construct
  REGION 1..710: note = Full-length modified heavy chain (version 1) of ScFv-HC-IgG4
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIKGGGGSGGGGSGGGGSGGGGSQVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSS

SEQ ID NO: 136 - protein, 218 aa, synthetic construct; identical to SEQ ID NO: 131
  REGION 1..218: note = Full-length light chain of ScFv-HC-IgG4

SEQ ID NO: 137 - protein, 713 aa, synthetic construct
  REGION 1..713: note = Full-length modified heavy chain (version 2) of ScFv-HC-IgG1-LALA
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 138 - protein, 713 aa, synthetic construct
  REGION 1..713: note = Full-length modified heavy chain (version 1) of ScFv-HC-IgG1-LALA
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIKGGGGSGGGGSGGGGSGGGGSQVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSS

SEQ ID NO: 139 - protein, 218 aa, synthetic construct; identical to SEQ ID NO: 131
  REGION 1..218: note = Full-length light chain of ScFv-HC-IgG1-LALA

SEQ ID NO: 140 - protein, 449 aa, synthetic construct
  REGION 1..449: note = Full length of PD1-1A7-H2K3-IgG4 heavy chain
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 141 - protein, 218 aa, synthetic construct; identical to SEQ ID NO: 131
  REGION 1..218: note = Full length of PD1-1A7-H2K3-IgG4 light chain

SEQ ID NO: 142 - protein, 441 aa, synthetic construct
  REGION 1..441: note = Full length of CD40-6A7-H4K2-IgG2 heavy chain
QVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 143 - protein, 219 aa, synthetic construct
  REGION 1..219: note = Full length of CD40-6A7-H4K2-IgG2 light chain
DVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 452 aa, synthetic construct
  REGION 1..452: note = Full length of Selicrelumab heavy chain
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 145 - protein, 214 aa, synthetic construct
  REGION 1..214: note = Full length of Selicrelumab light chain
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 107 aa, synthetic construct [sequence omitted]

SEQ ID NO: 147 - protein, 330 aa, synthetic construct
  REGION 1..330: note = IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 330 aa, synthetic construct
  REGION 1..330: note = IgG1 heavy chain constant region with LALA mutations
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 327 aa, synthetic construct
  REGION 1..327: note = IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 150 - protein, 327 aa, synthetic construct
  REGION 1..327: note = IgG4 constant region with KIH mutations
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 151 - protein, 233 aa, synthetic construct
  REGION 1..233: note = IgG4 Fc (CH1 region deleted) region with KIH mutations
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 152 - protein, 23 aa, synthetic construct
  REGION 1..23: note = Linker peptide sequence-1
ASTGGGGSGGGGSGGGGSGGGGS

SEQ ID NO: 153 - protein, 15 aa, synthetic construct
  REGION 1..15: note = Linker peptide sequence-2
GGGGSGGGGSGGGGS

SEQ ID NO: 154 - protein, 20 aa, synthetic construct
  REGION 1..20: note = Linker peptide sequence-3
GGGGSGGGGSGGGGSGGGGS

SEQ ID NO: 155 - protein, 250 aa, synthetic construct
  REGION 1..250: note = CD40 ScFV-1 (VH-VL) version 1
QVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSSASTGGGGSGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIK

SEQ ID NO: 156 - protein, 250 aa, synthetic construct
  REGION 1..250: note = CD40 ScFV-2 (VH-VL) version 2
QVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSSASTGGGGSGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIK

SEQ ID NO: 157 - protein, 247 aa, synthetic construct
  REGION 1..247: note = CD40 ScFV-3 (VL-VH version 2
DVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 158 - protein, 247 aa, synthetic construct
  REGION 1..247: note = CD40 ScFV-4 (VL-VH) version 1
DVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIKGGGGSGGGGSGGGGSGGGGSQVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSS

SEQ ID NO: 159 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 160 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 161 - protein, 711 aa, synthetic construct
  REGION 1..711: note = PD1-C40-6A7-FV3A (heavy chain)
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSSGGGGSGGGGSVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 162 - protein, 716 aa, synthetic construct
  REGION 1..716: note = Toripalimab-6A7-HC-IgG1-LALA (heavy chain)
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 163 - protein, 219 aa, synthetic construct
  REGION 1..219: note = Toripalimab-6A7-HC-IgG1-LALA (light chain)
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 711 aa, synthetic construct
  REGION 1..711: note = Pembrolizumab-6A7-HC-IgG1-LALA (heavy chain)
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 165 - protein, 218 aa, synthetic construct
  REGION 1..218: note = Pembrolizumab-6A7-HC-IgG1-LALA (light chain)
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 449 aa, synthetic construct; identical to SEQ ID NO: 130
  REGION 1..449: note = 1A7-selicrelumab-FVKH-IgG4 PD-1 arm (heavy chain)

SEQ ID NO: 167 - protein, 218 aa, synthetic construct; identical to SEQ ID NO: 131
  REGION 1..218: note = 1A7-selicrelumab-FVKH-IgG4 (light chain)

SEQ ID NO: 168 - protein, 717 aa, synthetic construct
  REGION 1..717: note = 1A7-selicrelumab-FV3A-IgG4 (heavy chain)
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 169 - protein, 218 aa, synthetic construct; identical to SEQ ID NO: 131
  REGION 1..218: note = 1A7-selicrelumab-FV3A-IgG4 (light chain)

SEQ ID NO: 170 - protein, 564 aa, synthetic construct
  REGION 1..564: note = 1A7-selicrelumab-DART-IgG4 (heavy chain)
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGSGGGGEVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 171 - protein, 352 aa, synthetic construct
  REGION 1..352: note = 1A7-selicrelumab-DART-IgG4 (light chain)
DNVLTQSPATLSVSPGERATLSCKASQSVDFDGDSYMNWYQQKPGQPPRLLIYAASNLESGIPARFSGSGSGTDFTLTISSVEPEDFATYYCQQSNEDPPTFGGGTKVEIKGGGSGGGGQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 716 aa, synthetic construct
  REGION 1..716: note = 1A7-selicrelumab-FVHC-IgG4 (heavy chain)
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 173 - protein, 486 aa, synthetic construct
  REGION 1..486: note = 1A7-selicrelumab-FVKH-IgG4 CD40 arm (heavy chain)
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 174 - protein, 352 aa, synthetic construct; identical to SEQ ID NO: 171
  REGION 1..352: note = 1A7-selicrelumab-FVHC-IgG4(light chain)

SEQ ID NO: 175 - protein, 714 aa, synthetic construct
  REGION 1..714: note = Pembrolizumab-Seli-FVHC-IgG4(heavy chain)
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 176 - protein, 218 aa, synthetic construct; identical to SEQ ID NO: 165
  REGION 1..218: note = Pembrolizumab-Seli-FVHC-IgG4(light chain)

SEQ ID NO: 177 - protein, 710 aa, synthetic construct
  REGION 1..710: note = Pembrolizumab-APX005M-FVHC-IgG4(heavy chain)
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGGGSGGGGSGGGGSDIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKGGGGSGGGGSGGGGSGGGGSQVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 178 - protein, 218 aa, synthetic construct; identical to SEQ ID NO: 165
  REGION 1..218: note = Pembrolizumab-APX005M-FVHC-IgG4(light chain)

SEQ ID NO: 179 - protein, 117 aa, synthetic construct [sequence omitted]

SEQ ID NO: 180 - protein, 107 aa, synthetic construct [sequence omitted]

SEQ ID NO: 181 - protein, 125 aa, synthetic construct [sequence omitted]

SEQ ID NO: 182 - protein, 112 aa, synthetic construct [sequence omitted]

SEQ ID NO: 183 - protein, 120 aa, synthetic construct
  REGION 1..120: note = Sintilimab VH
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 184 - protein, 107 aa, synthetic construct
  REGION 1..107: note = Sintilimab VL
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 185 - protein, 116 aa, synthetic construct
  REGION 1..116: note = Camrelizumab VH
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 186 - protein, 107 aa, synthetic construct
  REGION 1..107: note = Camrelizumab VL
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 187 - protein, 118 aa, synthetic construct
  REGION 1..118: note = Tislelizumab VH
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 188 - protein, 107 aa, synthetic construct
  REGION 1..107: note = Tislelizumab VL
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 189 - protein, 120 aa, synthetic construct
  REGION 1..120: note = RMP14 VH
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 190 - protein, 106 aa, synthetic construct
  REGION 1..106: note = RMP14 VL
DTVLTQSPALAVSPGERVTISCRASDSVSTLMHWYQQKPGQQPKLLIYLASHLESGVPARFSGSGSGTDFTLTIDPVEADDTATYYCQQSWNDPWTFGGGTKLELK

SEQ ID NO: 191 - protein, 120 aa, synthetic construct
  REGION 1..120: note = APX005M VH
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 192 - protein, 108 aa, synthetic construct
  REGION 1..108: note = APX005M VL
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 193 - protein, 116 aa, synthetic construct
  REGION 1..116: note = FGK4.5 VH (mouse)
EVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSS

SEQ ID NO: 194 - protein, 106 aa, synthetic construct; identical to SEQ ID NO: 190
  REGION 1..106: note = FGK4.5 VL

SEQ ID NO: 195 - protein, 116 aa, synthetic construct
  REGION 1..116: note = Anti-mCD40 VH
VQLQQSGGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGTTVTTVSS

SEQ ID NO: 196 - protein, 109 aa, synthetic construct
  REGION 1..109: note = Anti-mCD40 VL
DIELTQSPSTLPASLGERVTISCRASQSISNSLNWYQQKPDGTVKRLIYSTSTLESGVPSRFSGSGSGTDYSLSISSLESEDFAMYYCLQYATYPHTFGSGTKLEIKRC

SEQ ID NO: 197 - protein, 119 aa, synthetic construct
  REGION 1..119: note = Mitazalimab VH
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSS

SEQ ID NO: 198 - protein, 112 aa, synthetic construct
  REGION 1..112: note = Mitazalimab VL
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 199 - protein, 126 aa, synthetic construct
  REGION 1..126: note = selicrelumab VH
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 200 - protein, 107 aa, synthetic construct
  REGION 1..107: note = selicrelumab VL
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 201 - protein, 721 aa, synthetic construct
  REGION 1..721: note = Selicrelumab-1A7-FVHC-IgG4 (heavy chain)
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGGGSGGGGSGGGGSDNVLTQSPATLSVSPGERATLSCKASQSVDFDGDSYMNWYQQKPGQPPRLLIYAASNLESGIPARFSGSGSGTDFTLTISSVEPEDFATYYCQQSNEDPPTFGGGTKVEIKGGGGSGGGGSGGGGSGGGGSEVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSS

SEQ ID NO: 202 - protein, 214 aa, synthetic construct; identical to SEQ ID NO: 145
  REGION 1..214: note = Selicrelumab-1A7-FVHC-IgG4 (light chain)

SEQ ID NO: 203 - protein, 615 aa, synthetic construct
  REGION 1..615: note = SdAb-6A7-FVHC-IgG1-LALA (heavy chain)
QVQLVESGGGLVQPGGSLRLSCAASGGTLDYYAIGWFRQAPGKEREAVSCISSSDGSTYYADSVKGRFTISRDNSKNTLYLQMNSLRAEDTAVYHCATDRACGSSWLGAESWAQGTLVTVSSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 204 - protein, 352 aa, synthetic construct
  REGION 1..352: note = SdAb VHH
QVQLVESGGGLVQPGGSLRLSCAASGGTLDYYAIGWFRQAPGKEREAVSCISSSDGSTYYADSVKGRFTISRDNSKNTLYLQMNSLRAEDTAVYHCATDRACGSSWLGAESWAQGTLVTVSSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 205 - protein, 8 aa, synthetic construct
  REGION 1..8: note = Linker peptide sequence-4
GGGSGGGG

SEQ ID NO: 206 - protein, 253 aa, synthetic construct
  REGION 1..253: note = selicrelumab ScFV
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 207 - protein, 120 aa, synthetic construct [sequence omitted]

SEQ ID NO: 208 - protein, 111 aa, synthetic construct [sequence omitted]

SEQ ID NO: 209 - protein, 113 aa, synthetic construct [sequence omitted]

SEQ ID NO: 210 - protein, 107 aa, synthetic construct [sequence omitted]

SEQ ID NO: 211 - protein, 118 aa, synthetic construct [sequence omitted]

SEQ ID NO: 212 - protein, 107 aa, synthetic construct [sequence omitted]

SEQ ID NO: 213 - protein, 120 aa, synthetic construct
  REGION 1..120: note = Avelumab VH
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 214 - protein, 110 aa, synthetic construct
  REGION 1..110: note = Avelumab VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 215 - protein, 709 aa, synthetic construct
  REGION 1..709: note = Atezolizumab-6A7-FVHC-IgG1-LALA (heavy chain)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 216 - protein, 214 aa, synthetic construct
  REGION 1..214: note = Atezolizumab-6A7-FVHC-IgG1-LALA (light chain)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 711 aa, synthetic construct
  REGION 1..711: note = Avelumab-6A7-FVHC-IgG1-LALA (heavy chain)
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 218 - protein, 216 aa, synthetic construct
  REGION 1..216: note = Avelumab-6A7-FVHC-IgG1-LALA (light chain)
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2023/0220082 A1
(43) Pub. Date: Jul. 13, 2023

(54) ANTI-PD-1/CD40 BISPECIFIC ANTIBODIES AND USES THEREOF

(71) Applicant: Eucure (Beijing) Biopharma Co., Ltd., Beijing (CN)

(72) Inventors: Baihong Liu, Beijing (CN); Yi Yang, Beijing (CN); Yuelei Shen, Beijing (CN)

(21) Appl. No.: 18/150,428

(22) Filed: Jan. 5, 2023

Related U.S. Application Data
(63) Continuation of application No. PCT/CN2021/123438, filed on Oct. 13, 2021.

(30) Foreign Application Priority Data
Oct. 14, 2020 (WO) PCT/CN2020/120918
Apr. 2, 2021 (WO) PCT/CN2021/085335

(51) Int. Cl. C07K 16/28; A61P 35/00; A61P 1/00; A61P 17/00

(52) U.S. Cl. CPC C07K 16/2818; C07K 16/2878; A61P 35/00; A61P 1/00; A61P 17/00; C07K 2317/31; C07K 2317/55; C07K 2317/622; C07K 2317/92; A61K 2039/505

(57) ABSTRACT

This disclosure relates to antigen-binding protein constructs (e.g., bispecific antibodies or antigen-binding fragments thereof), wherein the antigen-binding protein constructs specifically bind to two different antigens (e.g., PD-1 and CD40).

### CROSS REFERENCE TO RELATED APPLICATIONS

Pursuant to 35 U.S.C. 21 119(e), this application is a continuation of International Application No. PCT/CN2021/123438, filed on Oct. 13, 2021, which claims the benefit of PCT Application No. PCT/CN2020/120918, filed on Oct. 14, 2020, and PCT Application No. PCT/CN2021/085335, filed on Apr. 2, 2021. The entire contents of the foregoing applications are incorporated herein by reference.

### SEQUENCE LISTING

This application contains a Sequence Listing that has been submitted electronically as an XML file named 45124-0029001_SL_ST26.xml. The XML file, created on Dec. 30, 2022, is 303,796 bytes in size. The material in the XML file is hereby incorporated by reference in its entirety.

### TECHNICAL FIELD

This disclosure relates to antigen-binding protein constructs (e.g., bispecific antibodies or antigen-binding fragments thereof).

### BACKGROUND

A bispecific antibody is an artificial protein that can simultaneously bind to two different types of antigens or two different epitopes. This dual specificity opens up a wide range of applications, including redirecting T cells to tumor cells, dual targeting of different disease mediators, and delivering payloads to targeted sites. The approval of catumaxomab (anti-EpCAM and anti-CD3) and blinatumomab (anti-CD19 and anti-CD3) has become a major milestone in the development of bispecific antibodies.

As bispecific antibodies have various applications. There is a need to continue to develop various therapeutics based on bispecific antibodies.

### SUMMARY

This disclosure relates to antigen-binding protein constructs, wherein the antigen-binding protein construct specifically bind to two or more different antigens (e.g., PD-1 and CD40). In some embodiments, the antigen-binding protein constructs are bispecific antibodies targeting both PD-1 and CD40. In some embodiments, the bispecific antibodies described herein is effective in treating cancer by multiple mechanisms. For example, the bispecific antibodies can block the PD-1/PD-L1 pathway thereby activating the immune response. In addition, the bispecific antibodies can bridge T cells and APC cells, to facilitate antigen-presenting and activate CD40 pathway in APC cells. Further, the bispecific antibodies can activate CD40 pathway in a PD-1 dependent manner, thereby amplifying immune response signals in tumor microenvironment or a tumor-draining lymph node. This mechanism can also reduce overall immune activation and reduce side effects, e.g., toxicity in liver. In some embodiments, the bispecific antibodies described herein cannot activate CD40 pathway via Fc receptor-mediated (e.g., FCγRIM-mediated) CD40 clustering, thereby further reducing toxicity e.g., in tissues expressing high level of FCγRIIB, such as liver.

In one aspect, the disclosure provides an antigen-binding protein construct, comprising a first antigen-binding site that specifically binds to PD-1, and a second antigen-binding site that specifically binds to CD40.

In some embodiments, the antigen-binding protein construct induces CD40 pathway activities when the antigen-binding protein construct binds to a cell expressing PD-1. In some embodiments, the antigen-binding protein construct induces CD40 pathway activities in the presence of one or more cells expressing PD-1. In some embodiments, the antigen-binding protein construct induces CD40 pathway activities in a tumor microenvironment or a tumor-draining lymph node. In some embodiments, the antigen-binding protein construct does not induce CD40 pathway activities in the absence of one or more cells expressing PD-1. In some embodiments, the antigen-binding protein construct is capable of activating CD40 pathway, wherein the activation of CD40 pathway depends on the binding of the antigen-binding protein construct to a cell expressing PD-1.

In some embodiments, the first antigen-binding site binds to a cell expressing PD-1. In some embodiments, the cell expressing PD-1 is a T cell, a NK cell, or a bone marrow cell (e.g., a T cell). In some embodiments, the cell expressing PD-1 is a T cell. In some embodiments, the cell is a myeloid cell (e.g., a macrophage, a Myeloid-derived suppressor cell (MDSC), or a T cell).

In some embodiments, the second antigen-binding site binds to a cell expressing CD40. In some embodiments, the cell expressing CD40 is an antigen-presenting cell. In some embodiments, the antigen-binding protein construct comprises an Fc region. In some embodiments, the second antigen-binding site is linked to the Fc region. In some embodiments, the second antigen-binding site is linked to the C-terminal of the Fc region. In some embodiments, the antigen-binding protein construct is incapable of activating CD40 through Fc receptor (e.g., FCγRIIB)-mediated activity or Fc receptor (e.g., FCγRIIB)-mediated clustering. In some embodiments, the antigen-binding protein construct is incapable of inducing Fc receptor (e.g., FCγRIIB)-mediated clustering.

In some embodiments, the antigen-binding protein construct is a bispecific antibody.

In some embodiments, the first antigen-binding site that specifically binds to PD-1 comprises a ScFv or a VHH domain. In some embodiments, the antigen binding site that specifically binds to PD-1 comprises a PD-1 ligand (e.g., PD-L1) or a soluble portion thereof In some embodiments, the second antigen-binding site that specifically binds to CD40 comprises a ScFv or a VHH domain. In some embodiments, the second antigen binding site that specifically binds to CD40 comprises a CD40 ligand (e.g., CD40L) or a soluble portion thereof.

In some embodiments, the first antigen binding site comprises a heavy chain variable region and a light chain variable region as described herein. In some embodiments, the second antigen binding site comprises a heavy chain variable region and a light chain variable region as described herein.

In some embodiments, the PD-1 ligand comprises a sequence that is at least 80%, 85%, 90%, 95%, or 100% identical to amino acids 19 — 238 of SEQ ID NO: 159. In some embodiments, the CD40 ligand comprises a sequence that is at least 80%, 85%, 90%, 95%, or 100% identical to amino acids 47 — 261 of SEQ ID NO: 160.

In one aspect, the disclosure is related to an antigen-binding protein construct, comprising a first heavy chain variable region and a first light chain variable region, in some embodiments, the first heavy chain variable region and the first light chain variable region associate with each other, forming a first antigen binding site that specifically binds to PD-1; and a second heavy chain variable region and a second light chain variable region, in some embodiments, the second heavy chain variable region and the second light chain variable region associate with each other, forming a second antigen binding site that specifically binds to CD40.

In some embodiments, the antigen-binding protein construct comprises a first polypeptide comprising the first heavy chain variable region, a first heavy chain constant region 2 (CH2), and a first heavy chain constant region 3 (CH3); and a second polypeptide comprising the second heavy chain variable region, a second heavy chain constant region 2 (CH2), and a second heavy chain constant region 3 (CH3).

In some embodiments, the antigen-binding protein construct comprises a third polypeptide comprising the first light chain variable region; and a fourth polypeptide comprising the second light chain variable region.

In some embodiments, the second polypeptide further comprises the second light chain variable region.

In some embodiments, the antigen-binding protein construct comprises a third polypeptide comprising the first light chain variable region.

In some embodiments, the first polypeptide further comprises the first light chain variable region.

In some embodiments, the antigen-binding protein construct comprises a first polypeptide comprising the first heavy chain variable region, the second heavy chain variable region, and the second light chain variable region; and a second polypeptide comprising the first light chain variable region.

In some embodiments, the first polypeptide further comprises a heavy chain constant region 1 (CH1), a heavy chain constant region 2 (CH2), and a heavy chain constant region 3 (CH3).

In some embodiments, the first heavy chain variable region (VH1) comprising complementarity determining regions (CDRs) 1, 2, and 3. In some embodiments, the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprising CDRs 1, 2, and 3. In some embodiments, the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence. In some embodiments, the selected VH1 CDRs 1, 2, and 3 amino acid sequences, the selected VL1 CDRs 1, 2, and 3 amino acid sequences are one of the following:

(1) the selected VH1 CDRs 1, 2, 3 amino acid sequences are set forth in SEQ ID NOs: 1, 2, and 3, respectively, and the selected VL1 CDRs 1, 2, 3 amino acid sequences are set forth in SEQ ID NOs: 4, 5, and 6, respectively, according to the Kabat numbering scheme;

(2) the selected VH1 CDRs 1, 2, 3 amino acid sequences are set forth in SEQ ID NOs: 19, 20, and 21, respectively, and the selected VL1 CDRs 1, 2, 3 amino acid sequences are set forth in SEQ ID NOs: 22, 23, and 24, respectively, according to the Chothia numbering scheme;

(3) the selected VH1 CDRs 1, 2, 3 amino acid sequences are set forth in SEQ ID NOs: 7, 8, and 9, respectively, and the selected VL1 CDRs 1, 2, 3 amino acid sequences are set forth in SEQ ID NOs: 10, 11, and 12, respectively, according to the Kabat numbering scheme;

(4) the selected VH1 CDRs 1, 2, 3 amino acid sequences are set forth in SEQ ID NOs: 25, 26, and 27, respectively, and the selected VL1 CDRs 1, 2, 3 amino acid sequences are set forth in SEQ ID NOs: 28, 29, and 30, respectively, according to the Chothia numbering scheme;

(5) the selected VH1 CDRs 1, 2, 3 amino acid sequences are set forth in SEQ ID NOs: 13, 14, and 15, respectively, and the selected VL1 CDRs 1, 2, 3 amino acid sequences are set forth in SEQ ID NOs: 16, 17, and 18, respectively, according to the Kabat numbering scheme; and

(6) the selected VH1 CDRs 1, 2, 3 amino acid sequences are set forth in SEQ ID NOs: 31, 32, and 33, respectively, and the selected VL1 CDRs 1, 2, 3 amino acid sequences are set forth in SEQ ID NOs: 34, 35, and 36, respectively, according to the Chothia numbering scheme.

In some embodiments, the second heavy chain variable region (VH2) comprising CDRs 1, 2, and 3. In some embodiments, the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprising CDRs 1, 2, and 3. In some embodiments, the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence. In some embodiments, the selected VH2 CDRs 1, 2, and 3 amino acid sequences, and the selected VL2 CDRs 1, 2, and 3 amino acid sequences are one of the following:

(1) the selected VH2 CDRs 1, 2, 3 amino acid sequences are set forth in SEQ ID NOs: 59, 60, and 61, respectively, and the selected VL2 CDRs 1, 2, 3 amino acid sequences are set forth in SEQ ID NOs: 62, 63, and 64, respectively, according to the Kabat numbering scheme;

(2) the selected VH2 CDRs 1, 2, 3 amino acid sequences are set forth in SEQ ID NOs: 77, 78, and 79, respectively, and the selected VL2 CDRs 1, 2, 3 amino acid sequences are set forth in SEQ ID NOs: 80, 81, and 82, respectively, according to the Chothia numbering scheme;

(3) the selected VH2 CDRs 1, 2, 3 amino acid sequences are set forth in SEQ ID NOs: 65, 66, and 67, respectively, and the selected VL2 CDRs 1, 2, 3 amino acid sequences are set forth in SEQ ID NOs: 68, 69, and 70, respectively, according to the Kabat numbering scheme;

(4) the selected VH2 CDRs 1, 2, 3 amino acid sequences are set forth in SEQ ID NOs: 83, 84, and 85, respectively, and the selected VL2 CDRs 1, 2, 3 amino acid sequences are set forth in SEQ ID NOs: 86, 87, and 88, respectively, according to the Chothia numbering scheme;

(5) the selected VH2 CDRs 1, 2, 3 amino acid sequences are set forth in SEQ ID NOs: 71, 72, and 73, respectively, and the selected VL2 CDRs 1, 2, 3 amino acid sequences are set forth in SEQ ID NOs: 74, 75, and 76, respectively, according to the Kabat numbering scheme; and

(6) the selected VH2 CDRs 1, 2, 3 amino acid sequences are set forth in SEQ ID NOs: 89, 90, and 91, respectively, and the selected VL2 CDRs 1, 2, 3 amino acid sequences are set forth in SEQ ID NOs: 92, 93, and 94, respectively, according to the Chothia numbering scheme.

In some embodiments, the disclosure is related to the antigen-binding protein construct that

(1) the selected VH1 CDRs 1, 2, 3 amino acid sequences are set forth in SEQ ID NOs: 1, 2, and 3, respectively, and the selected VL1 CDRs 1, 2, 3 amino acid sequences are set forth in SEQ ID NOs: 4, 5, and 6, respectively, the selected VH2 CDRs 1, 2, 3 amino acid sequences are set forth in SEQ ID NOs: 65, 66, and 67, respectively, and the selected VL2 CDRs 1, 2, 3 amino acid sequences are set forth in SEQ ID NOs: 68, 69, and 70, respectively, according to the Kabat numbering scheme; or

(2) the selected VH1 CDRs 1, 2, 3 amino acid sequences are set forth in SEQ ID NOs: 19, 20, and 21, respectively, and the selected VL1 CDRs 1, 2, 3 amino acid sequences are set forth in SEQ ID NOs: 22, 23, and 24, respectively, the selected VH2 CDRs 1, 2, 3 amino acid sequences are set forth in SEQ ID NOs: 83, 84, and 85, respectively, and the selected VL2 CDRs 1, 2, 3 amino acid sequences are set forth in SEQ ID NOs: 86, 87, and 88, respectively, according to the Chothia numbering scheme.

In some embodiments, the first heavy chain variable region comprises a sequence that is at least 80%, 85%, 90%, or 95% identical to SEQ ID NO: 40, 41, 42, or 53, and the first light chain variable region comprises a sequence that is at least 80%, 85%, 90%, or 95% identical to SEQ ID NO: 43, 44, 45, or 54.

In some embodiments, the first heavy chain variable region comprises a sequence that is at least 80%, 85%, 90%, or 95% identical to SEQ ID NO: 46, 47, 48, 49, or 55, and the first light chain variable region comprises a sequence that is at least 80%, 85%, 90%, or 95% identical to SEQ ID NO: 50, 51, 52, or 56.

In some embodiments, the first heavy chain variable region comprises a sequence that is at least 80%, 85%, 90%, or 95% identical to SEQ ID NO: 57, and the first light chain variable region comprises a sequence that is at least 80%, 85%, 90%, or 95% identical to SEQ ID NO: 58.

In some embodiments, the second heavy chain variable region comprises a sequence that is at least 80%, 85%, 90%, or 95% identical to SEQ ID NO: 98, 99, 100, or 120, and the second light chain variable region comprises a sequence that is at least 80%, 85%, 90%, or 95% identical to SEQ ID NO: 101, 102, 103, 104, or 121.

In some embodiments, the second heavy chain variable region comprises a sequence that is at least 80%, 85%, 90%, or 95% identical to SEQ ID NO: 105, 106, 107, 108, 122, 126, or 128, and the second light chain variable region comprises a sequence that is at least 80%, 85%, 90%, or 95% identical to SEQ ID NO: 109, 110, 111, 123, 127, or 129.

In some embodiments, the second heavy chain variable region comprises a sequence that is at least 80%, 85%, 90%, or 95% identical to SEQ ID NO: 112, 113, 114, 115, or 124, and the second light chain variable region comprises a sequence that is at least 80%, 85%, 90%, or 95% identical to SEQ ID NO: 116, 117, 118, 119, or 125.

In some embodiments, the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 45, the second heavy chain variable region comprises a sequence that is at least 80%, 85%, 90%, or 95% identical to SEQ ID NO: 108 or 126, and the second light chain variable region comprises a sequence that is at least 80%, 85%, 90%, or 95% identical to SEQ ID NO: 110 or 127.

In some embodiments, the first polypeptide comprises a sequence that is at least 80%, 85%, 90%, or 95% identical to SEQ ID NO: 130, and the third polypeptide comprise a sequence that is at least 80%, 85%, 90%, or 95% identical to SEQ ID NO: 131.

In some embodiments, the second polypeptide comprises a sequence that is at least 80%, 85%, 90%, or 95% identical to SEQ ID NO: 132 or 133.

In some embodiments, the first polypeptide comprises a sequence that is at least 80%, 85%, 90%, or 95% identical to SEQ ID NO: 130; the second polypeptide comprises a sequence that is at least 80%, 85%, 90%, or 95% identical to SEQ ID NO: 132 or 133; and the third polypeptide comprises a sequence that is at least 80%, 85%, 90%, or 95% identical to SEQ ID NO: 131.

In some embodiments, the first polypeptide comprises a sequence that is at least 80%, 85%, 90%, or 95% identical to SEQ ID NO: 134 or 135, and the second polypeptide comprises a sequence that is at least 80%, 85%, 90%, or 95% identical to SEQ ID NO: 136.

In some embodiments, the first polypeptide comprises a sequence that is at least 80%, 85%, 90%, or 95% identical to SEQ ID NO: 137 or 138, and the second polypeptide comprises a sequence that is at least 80%, 85%, 90%, or 95% identical to SEQ ID NO: 139.

In some embodiments, the antigen-binding protein construct is a bispecific antibody.

In some embodiments, the antigen-binding protein construct is a Fab-scFv-Fc.

In some embodiments, the antigen-binding protein construct is a TrioMab, a bispecific antibody with a common light chain, a CrossMab, a 2:1 CrossMab, a 2:2 CrossMab, a Duobody, a Dual-variable-domain antibody (DVD-Ig), a scFv-IgG, a IgG-IgG format antibody, a Fab-scFv-Fc format antibody, a TF, an ADAPTIR, a Bispecific T cell Engager (BiTE), a BiTE-Fc, a Dual affinity retargeting (DART), a DART-Fc (a DART with Fc), a tetravalent DART, a Tandem diabody (TandAb), a scFv-scFv-scFv, an ImmTAC, a Tri-specific nanobody, or a Trispecific Killer Engager (TriKE).

In some embodiments, the antigen binding protein construct comprises one or more heavy chain constant domains from IgG1 or IgG4.

In some embodiments, the one or more heavy chain constant domain comprises LALA mutations and/or knob-into-hole (KIH) mutations.

In one aspect, the disclosure is related to a bispecific antibody or antigen-binding fragment thereof, comprising a first heavy chain polypeptide comprising a first heavy chain variable region; a first light chain polypeptide comprising a first light chain variable region; a second heavy chain polypeptide comprising a second heavy chain variable region; and a second light chain polypeptide, comprising a second light chain variable region.

In some embodiments, the first heavy chain variable region and the first light chain variable region associate with each other, forming a first antigen binding site that specifically binds to PD-1, and the second heavy chain variable region and the second light chain variable region associate with each other, forming a second antigen binding site that specifically binds to CD40.

In some embodiments, the disclosure is related to the bispecific antibody or antigen-binding fragment thereof that comprises the first heavy chain variable region (VH1) comprising complementarity determining regions (CDRs) 1, 2, and 3, in some embodiments, the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; the first light chain variable region (VL1) comprising CDRs 1, 2, and 3, in some embodiments, the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence; the second heavy chain variable region (VH2) comprising complementarity determining regions (CDRs) 1, 2, and 3, in some embodiments, the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprising CDRs 1, 2, and 3, in some embodiments, the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence.

In some embodiments, the selected VH1 CDRs 1, 2, and 3 amino acid sequences, the selected VL1 CDRs 1, 2, and 3 amino acid sequences, the selected VH2 CDRs 1, 2, and 3 amino acid sequences, and the selected VL2 CDRs 1, 2, and 3 amino acid sequences are one of the following:

(1) the selected VH1 CDRs 1, 2, 3 amino acid sequences are set forth in SEQ ID NOs: 1, 2, and 3, respectively, and the selected VL1 CDRs 1, 2, 3 amino acid sequences are set forth in SEQ ID NOs: 4, 5, and 6, respectively, the selected VH2 CDRs 1, 2, 3 amino acid sequences are set forth in SEQ ID NOs: 65, 66, and 67, respectively, and the selected VL2 CDRs 1, 2, 3 amino acid sequences are set forth in SEQ ID NOs: 68, 69, and 70, respectively, according to the Kabat numbering scheme; and

(2) the selected VH1 CDRs 1, 2, 3 amino acid sequences are set forth in SEQ ID NOs: 19, 20 and 21, respectively, and the selected VL1 CDRs 1, 2, 3 amino acid sequences are set forth in SEQ ID NOs: 22, 23, and 24, respectively, the selected VH2 CDRs 1, 2, 3 amino acid sequences are set forth in SEQ ID NOs: 83, 84, and 85, respectively, and the selected VL2 CDRs 1, 2, 3 amino acid sequences are set forth in SEQ ID NOs: 86, 87, and 88, respectively, according to the Chothia numbering scheme.

In some embodiments, the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 45, the second heavy chain variable region comprises a sequence that is at least 80%, 85%, 90%, or 95% identical to SEQ ID NO: 108 or 126, and the second light chain variable region comprises a sequence that is at least 80%, 85%, 90%, or 95% identical to SEQ ID NO: 110 or 127.

In some embodiments, the first heavy chain polypeptide comprises a sequence that is at least 80%, 85%, 90%, or 95% identical to SEQ ID NO: 130, and the first light chain polypeptide comprise a sequence that is at least 80%, 85%, 90%, or 95% identical to SEQ ID NO: 131.

In one aspect, the disclosure is related to a bispecific antibody or antigen-binding fragment thereof, comprising a heavy chain polypeptide comprising a first heavy chain variable region; a light chain polypeptide comprising a first light chain variable region; and a single-chain variable fragment polypeptide comprising a second heavy chain variable region, and a second light chain variable region. In some embodiments, the first heavy chain variable region and the first light chain variable region associate with each other, forming a first antigen binding site that specifically binds to PD-1, and the second heavy chain variable region and the second light chain variable region associate with each other, forming a second antigen binding site that specifically binds to CD40.

In some embodiments, the single-chain variable fragment polypeptide comprises from N-terminus to C-terminus: the second heavy chain variable region; a linker peptide sequence; the second light chain variable region; a heavy chain constant region 2 (CH2); and a heavy chain constant region 3 (CH3).

In some embodiments, the linker peptide sequence comprises a sequence that is at least 80% identical to ASTGGGGSGGGGSGGGGSGGGGS (SEQ ID NO: 152).

In some embodiments, the disclosure is related to the bispecific antibody or antigen-binding fragment thereof as described herein, comprising the first heavy chain variable region (VH1) comprising complementarity determining regions (CDRs) 1, 2, and 3, in some embodiments, the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; the first light chain variable region (VL1) comprising CDRs 1, 2, and 3, in some embodiments, the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence; the second heavy chain variable region (VH2) comprising complementarity determining regions (CDRs) 1, 2, and 3, in some embodiments, the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprising CDRs 1, 2, and 3, in some embodiments, the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence.

In some embodiments, the selected VH1 CDRs 1, 2, and 3 amino acid sequences, the selected VL1 CDRs 1, 2, and 3 amino acid sequences, the selected VH2 CDRs 1, 2, and 3 amino acid sequences, and the selected VL2 CDRs 1, 2, and 3 amino acid sequences are one of the following:

(1) the selected VH1 CDRs 1, 2, 3 amino acid sequences are set forth in SEQ ID NOs: 1, 2, and 3, respectively, and the selected VL1 CDRs 1, 2, 3 amino acid sequences are set forth in SEQ ID NOs: 4, 5, and 6, respectively, the selected VH2 CDRs 1, 2, 3 amino acid sequences are set forth in SEQ ID NOs: 65, 66, and 67, respectively, and the selected VL2 CDRs 1, 2, 3 amino acid sequences are set forth in SEQ ID NOs: 68, 69, and 70, respectively, according to the Kabat numbering scheme; and

(2) the selected VH1 CDRs 1, 2, 3 amino acid sequences are set forth in SEQ ID NOs: 19, 20, and 21, respectively, and the selected VL1 CDRs 1, 2, 3 amino acid sequences are set forth in SEQ ID NOs: 22, 23, and 24, respectively, the selected VH2 CDRs 1, 2, 3 amino acid sequences are set forth in SEQ ID NOs: 83, 84, and 85, respectively, and the selected VL2 CDRs 1, 2, 3 amino acid sequences are set forth in SEQ ID NOs: 86, 87, and 88, respectively, according to the Chothia numbering scheme.

In some embodiments, the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 45, the second heavy chain variable region comprises a sequence that is at least 80%, 85%, 90%, or 95% identical to SEQ ID NO: 108 or 126, and the second light chain variable region comprises a sequence that is at least 80%, 85%, 90%, or 95% identical to SEQ ID NO: 110 or 127.

In some embodiments, the disclosure is related to the bispecific antibody or antigen-binding fragment thereof that

(1) the heavy chain polypeptide comprises a sequence that is at least 80%, 85%, 90%, or 95% identical to SEQ ID NO: 130, and the light chain polypeptide comprise a sequence that is at least 80%, 85%, 90%, or 95% identical to SEQ ID NO: 131; and

(2) the single-chain variable fragment polypeptide comprises a sequence that is at least 80%, 85%, 90%, or 95% identical to SEQ ID NO: 132 or 133.

In one aspect, the disclosure is related to a bispecific antibody or antigen-binding fragment thereof, comprising a heavy chain polypeptide comprising a first heavy chain variable region, and a light chain polypeptide comprising a first light chain variable region. In some embodiments, a single-chain variable fragment polypeptide is linked to the C-terminus of the heavy chain polypeptide. In some embodiments, the single-chain variable fragment polypeptide comprises a second heavy chain variable region and a second light chain variable region. In some embodiments, the first heavy chain variable region and the first light chain variable region associate with each other, forming a first antigen binding site that specifically binds to PD-1, and the second heavy chain variable region and the second light chain variable region associate with each other, forming a second antigen binding site that specifically binds to CD40.

In some embodiments, the single-chain variable fragment polypeptide is linked to the first heavy chain polypeptide through a first linker peptide sequence.

In some embodiments, the first linker peptide sequence comprises a sequence that is at least 80% identical to GGGSGGGGSGGGGS (SEQ ID NO: 153).

In some embodiments, the single-chain variable fragment polypeptide comprises from N-terminus to C-terminus: the second light chain variable region; a second linker peptide sequence; and the second heavy chain variable region.

In some embodiments, the second linker peptide sequence comprises a sequence that is at least 80% identical to GGGGSGGGGSGGGGSGGGGS (SEQ ID NO: 154).

In some embodiments, the disclosure is related to the bispecific antibody or antigen-binding fragment thereof as described herein, comprising the first heavy chain variable region (VH1) comprising complementarity determining regions (CDRs) 1, 2, and 3, in some embodiments, the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; the first light chain variable region (VL1) comprising CDRs 1, 2, and 3, in some embodiments, the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence; the second heavy chain variable region (VH2) comprising complementarity determining regions (CDRs) 1, 2, and 3, in some embodiments, the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprising CDRs 1, 2, and 3, in some embodiments, the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence;

In some embodiments, the selected VH1 CDRs 1, 2, and 3 amino acid sequences, the selected VL1 CDRs 1, 2, and 3 amino acid sequences, the selected VH2 CDRs 1, 2, and 3 amino acid sequences, and the selected VL2 CDRs 1, 2, and 3 amino acid sequences are one of the following:

(1) the selected VH1 CDRs 1, 2, 3 amino acid sequences are set forth in SEQ ID NOs: 1, 2, and 3, respectively, and the selected VL1 CDRs 1, 2, 3 amino acid sequences are set forth in SEQ ID NOs: 4, 5, and 6, respectively, the selected VH2 CDRs 1, 2, 3 amino acid sequences are set forth in SEQ ID NOs: 65, 66, and 67, respectively, and the selected VL2 CDRs 1, 2, 3 amino acid sequences are set forth in SEQ ID NOs: 68, 69, and 70, respectively, according to the Kabat numbering scheme; and

(2) the selected VH1 CDRs 1, 2, 3 amino acid sequences are set forth in SEQ ID NOs: 19, 20, and 21, respectively, and the selected VL1 CDRs 1, 2, 3 amino acid sequences are set forth in SEQ ID NOs: 22, 23, and 24, respectively, the selected VH2 CDRs 1, 2, 3 amino acid sequences are set forth in SEQ ID NOs: 83, 84, and 85, respectively, and the selected VL2 CDRs 1, 2, 3 amino acid sequences are set forth in SEQ ID NOs: 86, 87, and 88, respectively, according to the Chothia numbering scheme.

In some embodiments, the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 45, the second heavy chain variable region comprises a sequence that is at least 80%, 85%, 90%, or 95% identical to SEQ ID NO: 108 or 126, and the second light chain variable region comprises a sequence that is at least 80%, 85%, 90%, or 95% identical to SEQ ID NO: 110 or 127.

In some embodiments, the disclosure is related to a bispecific antibody or antigen-binding fragment thereof as described herein that

(1) the heavy chain polypeptide comprises a sequence that is at least 80%, 85%, 90%, or 95% identical to SEQ ID NO: 134 or 135; and

(2) the light chain polypeptide comprises a sequence that is at least 80%, 85%, 90%, or 95% identical to SEQ ID NO: 136.

In some embodiments, the disclosure is related to a bispecific antibody or antigen-binding fragment thereof as described herein that

(1) the heavy chain polypeptide comprises a sequence that is at least 80%, 85%, 90%, or 95% identical to SEQ ID NO: 137 or 138; and

(2) the light chain polypeptide comprises a sequence that is at least 80%, 85%, 90%, or 95% identical to SEQ ID NO: 139.

In one aspect, the disclosure provides a bispecific antibody, comprising: an anti-PD-1 antibody comprising an Fc region, and an antigen binding site that specifically binds to CD40. In some embodiments, the antigen binding site is linked to the Fc region of the anti-PD-1 antibody.

In some embodiments, the bispecific antibody induces CD40 pathway activities in an immune cell in the presence of one or more cells expressing PD-1. In some embodiments, the antigen binding site that specifically binds to CD40 comprises a ScFv or a VHH domain. In some embodiments, the antigen binding site that specifically binds to CD40 comprises a CD40 ligand (e.g., CD40L) or a soluble portion thereof In some embodiments, the antigen binding site that specifically binds to CD40 is linked to the C-terminal of the Fc region. In some embodiments, the antigen binding site that specifically binds to CD40 is inserted in the Fc region (e.g., at the 3A site).

In one aspect, the disclosure provides an antigen-binding protein construct (e.g., a bispecific antibody or antigen-binding fragment thereof) comprising or consisting of an antibody comprising an Fc region, wherein the antibody specifically binds to a target, and an antigen binding site (e.g., one or two antigen binding sites) that specifically binds to CD40. In some embodiments, the antigen binding site is linked to the Fc region of the antibody.

In some embodiments, the antigen-binding protein construct induces CD40 pathway activities in an immune cell in the presence of one or more cells expressing the target. In some embodiments, the antigen-binding protein construct is capable of activating CD40 pathway. In some embodiments, the activation of CD40 pathway depends on the binding of the antigen-binding protein construct to a cell expressing the target. In some embodiments, the antigen-binding protein construct induces CD40 pathway activities in a tumor microenvironment or a tumor-draining lymph node. In some embodiments, the antigen-binding protein construct does not induce CD40 pathway activities in the absence of one or more cells expressing the target. In some embodiments, the antigen-binding protein construct is incapable of activating CD40 pathway through Fc receptor-mediated activity or Fc receptor-mediated clustering. In some embodiments, the antigen-binding protein construct is incapable of activating CD40 pathway in the absence of PD-1 expressing cells.

In some embodiments, the target is an immune checkpoint molecule. In some embodiments, the target is a cancer specific antigen or a cancer-associated antigen. In some embodiments, the target is PD-1. In some embodiments, the antigen binding site that specifically binds to CD40 comprises a ScFv or a VHH domain. In some embodiments, the antigen binding site that specifically binds to CD40 comprises a CD40 ligand (e.g., CD40L) or a soluble portion thereof. In some embodiments, the antigen binding site that specifically binds to CD40 is linked to the C-terminal of the Fc region. In some embodiments, the antigen binding site that specifically binds to CD40 is inserted in the Fc region (e.g., at the 3A site).

In one aspect, the disclosure is related to an antibody-drug conjugate comprising the antigen-binding protein construct as described herein, or the bispecific antibody or antigen-binding fragment as described herein, covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antigen-binding protein construct as described herein, the bispecific antibody or antigen-binding fragment as described herein, or the antibody-drug conjugate as described herein, to the subject.

In some embodiments, the subject has a solid tumor.

In some embodiments, the cancer is non-small cell lung cancer (NSCLC), squamous cell carcinoma of the head and neck (SCCHN), head and neck cancer, renal cell carcinoma (RCC), melanoma, bladder cancer, gastric cancer, urothelial cancer, Merkel-cell carcinoma, triple-negative breast cancer (TNBC), or colorectal carcinoma.

In some embodiments, the cancer is melanoma, pancreatic carcinoma, mesothelioma, or a hematological malignancy.

In some embodiments, the method further comprises administering an anti-CTLA4 antibody, an anti-Her2 antibody, or an antibody targeting a tumor-associated antigen (TAA), to the subject.

In some embodiments, the method further comprises administering a chemotherapy to the subject.

In one aspect, the disclosure is related to a method of decreasing the rate of tumor growth, the method comprising administering to a subject in need thereof an effective amount of a composition comprising the antigen-binding protein construct as described herein, the bispecific antibody or antigen-binding fragment as described herein, or the antibody-drug conjugate as described herein, to the subject.

In one aspect, the disclosure is related to a method of killing a tumor cell, the method comprising administering to a subject in need thereof an effective amount of a composition comprising the antigen-binding protein construct as described herein, the bispecific antibody or antigen-binding fragment as described herein, or the antibody-drug conjugate as described herein, to the subject.

In one aspect, the disclosure is related to a pharmaceutical composition comprising the antigen-binding protein construct as described herein, or the bispecific antibody or antigen-binding fragment as described herein, and a pharmaceutically acceptable carrier.

In one aspect, the disclosure is related to a pharmaceutical composition comprising the antibody drug conjugate as described herein, and a pharmaceutically acceptable carrier.

In one aspect, the disclosure is related to a bispecific or multi-specific antibody or antigen-binding fragment thereof, comprising: a single-chain variable fragment polypeptide that specifically binds to CD40. In some embodiments, the single-chain variable fragment polypeptide is linked to the C-terminus of a heavy chain polypeptide of the bispecific or multi-specific antibody or antigen-binding fragment thereof.

In some embodiments, the bispecific or multi-specific antibody or antigen-binding fragment thereof specifically binds to a cancer specific antigen or a cancer-associated antigen. In some embodiments, the bispecific or multi-specific antibody or antigen-binding fragment thereof specifically binds to an immune checkpoint molecule.

In one aspect, the disclosure provides a nucleic acid comprising a polynucleotide encoding any polypeptide as described herein. In some embodiments, the nucleic acid encodes a bispecific antibody. In some embodiments, the nucleic acid is cDNA.

In one aspect, the disclosure provides a vector comprising one or more of the nucleic acids described herein.

In one aspect, the disclosure provides a cell comprising the vector described herein. In some embodiments, the cell is a CHO cell. In one aspect, the disclosure provides a cell comprising one or more of the nucleic acids described herein.

As used herein, the term “antigen-binding protein construct” is (i) a single polypeptide that includes one or more antigen-binding domains or (ii) a complex of two or more polypeptides (e.g., the same or different polypeptides) that together form one or more antigen-binding domains. Non-limiting examples and aspects of antigen-binding protein constructs are described herein.

As used herein, the term “antigen-binding domain,” “antigen-binding region” or “antigen-binding site” refers to one or more protein domain(s) (e.g., formed by amino acids from a single polypeptide or formed by amino acids from two or more polypeptides (e.g., the same or different polypeptides)) that is capable of specifically binding to an antigen. In some embodiments, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by a VH -VL dimer. In some embodiments, an antigen-binding domain can include an alternative scaffold. In some embodiments, the antigen antigen-binding domain is a ligand (e.g., PD-L1, CD154, or the soluble portion thereof). For example, the antigen can be the PD-1 receptor, and the antigen-binding domain (e.g., the soluble portion of PD-L1) can bind specifically to the PD-1 receptor. In some embodiments, the antigen antigen-binding domain is a VHH domain. Non-limiting examples of antigen-binding domains are described herein.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an antigen or an epitope. Non-limiting examples of antibodies include: monoclonal antibodies, polyclonal antibodies, multi-specific antibodies (e.g., bi-specific antibodies), single-chain antibodies, chimeric antibodies, heavy chain antibodies, single-domain antibodies (nanobodies), human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)2, Fv fragments, and VHH. As used herein, the “VHH” is the variable domain of the heavy chain antibodies.

As used herein, the term “human antibody” refers to an antibody that is encoded by a nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “multispecific antigen-binding protein construct” is an antigen-binding protein construct that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multi-specific antigen-binding protein construct binds two different epitopes (i.e., a “bispecific antigen-binding protein construct”). In some aspects, a multi-specific antigen-binding protein construct binds three different epitopes (i.e., a “trispecific antigen-binding protein construct”). In some aspects, a multi-specific antigen-binding protein construct binds four different epitopes (i.e., a “quadspecific antigen-binding protein construct”). In some aspects, a multi-specific antigen-binding protein construct binds five different epitopes (i.e., a “quintspecific antigen-binding protein construct”). Each binding specificity may be present in any suitable valency. Non-limiting examples of multispecific antigen-binding protein constructs are described herein.

As used herein, the term “bispecific antibody” refers to an antibody that binds to two different epitopes. The epitopes can be on the same antigen or on different antigens.

As used herein, when referring to an antibody, the phrases “specifically binding” and “specifically binds” mean that the antibody interacts with its target molecule (e.g., PD-1) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a PD-1 molecule may be referred to as a PD-1-specific antibody or an anti-PD-1 antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic structure of bispecific antibody Fab-ScFV-IgG.

FIG. 1B is a schematic structure of bispecific antibody ScFV-HC-IgG.

FIG. 2A shows a gel electrophoresis result of Fab-ScFV-IgG4. M is marker. Fab-ScFV-IgG4 is in lane 2.

FIG. 2B shows a gel electrophoresis result of ScFV-HC-IgG4. M is marker. ScFV-HC-IgG4 is in lane 3.

FIG. 3 shows luminescence level in Jurkat-Luc-hPD-1 cells activated by anti-PD1 antibody 1A7-H2K3-IgG4 (PD-1), Fab-ScFV-IgG4, or ScFV-HC-IgG4. The antibodies blocked the PD1/PD-L1 pathway thereby activating the Jurkat-Luc-hPD-1 cells. The luminescence signal is expressed as relative light units (Rlu).

FIG. 4A shows luminescence level in Jurkat-Luc-hCD40 cells in the presence of CHO-K1-hPD1 and a selected antibody. The antibody was selected from a 1:1 ratio combination of anti-PD-1 antibody 1A7-H2K3-IgG4 and anti-CD40 antibody 6A7-H4K2-IgG2 (PD-1+CD40), Fab-ScFV-IgG4, and ScFV-HC-IgG4. The luminescence signal is expressed as relative light units (Rlu).

FIG. 4B shows luminescence level in Jurkat-Luc-hCD40 cells in the presence of a selected antibody. The antibody was selected from a 1:1 ratio combination of anti-PD-1 antibody 1A7-H2K3-IgG4 and anti-CD40 antibody 6A7-H4K2-IgG2 (PD-1 +CD40), Fab-ScFV-IgG4, and ScFV-HC-IgG4. The luminescence signal is expressed as relative light units (Rlu).

FIG. 5 shows luminescence level in Jurkat-Luc-hCD40 cells in the presence of CHO-K1-hFcγRIM and a selected antibody. The antibody was selected from anti-CD40 monoclonal antibody 6A7-H4K2-IgG2 (CD40), Fab-ScFV-IgG4, and ScFV-HC-IgG4. The luminescence signal is expressed as relative light units (Rlu).

FIG. 6A shows luminescence level in Jurkat-Luc-hCD40 cells in the presence of a selected antibody. The antibody was selected from anti-CD40 monoclonal antibody 6A7-H4K2-IgG2 (CD40), ScFV-HC-IgG4, and ScFV-HC-IgG1-LALA. The luminescence signal is expressed as relative light units (Rlu).

FIG. 6B shows luminescence level in Jurkat-Luc-hCD40 cells in the presence of Juakat-PD1 cells and a selected antibody. The antibody was selected from anti-CD40 monoclonal antibody 6A7-H4K2-IgG2 (CD40), ScFV-HC-IgG4, and ScFV-HC-IgG1-LALA. The luminescence signal is expressed as relative light units (Rlu).

FIG. 6C shows luminescence level in Jurkat-Luc-hCD40 cells in the presence of CHO-K1-hFcγRIIB cells and a selected antibody. The antibody was selected from anti-CD40 monoclonal antibody 6A7-H4K2-IgG2 (CD40), ScFV-HC-IgG4, and ScFV-HC-IgG1-LALA. The luminescence signal is expressed as relative light units (Rlu).

FIG. 7 is a graph showing body weight over time of B-hCD40 mice that were injected with mouse colon cancer cells MC38, and were treated with bispecific antibodies ScFV-HC-IgG4 or ScFV-HC-IgG1-LALA. Physiological saline (PS) solution was injected as a control.

FIG. 8 is a graph showing percentage change of body weight over time of B-hCD40 mice that were injected with mouse colon cancer cells MC38, and were treated with bispecific antibodies ScFV-HC-IgG4 or ScFV-HC-IgG1-LALA. Physiological saline (PS) solution was injected as a control.

FIG. 9 is a graph showing average tumor volume in different groups of B-hCD40 mice that were injected with mouse colon cancer cells MC38, and were treated with bispecific antibodies ScFV-HC-IgG4 or ScFV-HC-IgG1-LALA. Physiological saline (PS) solution was injected as a control.

FIG. 10 is a graph showing body weight over time of B-hPD-1/hCD40 mice that were injected with B16F10-hPD-L1 cells, and were treated with monospecific or bispecific antibodies. Physiological saline (PS) solution was injected as a control.

FIG. 11 is a graph showing percentage change of body weight over time of B-hPD-1/hCD40 mice that were injected with B16F10-hPD-L1 cells, and were treated with monospecific or bispecific antibodies. Physiological saline (PS) solution was injected as a control.

FIG. 12 is a graph showing average tumor volume in different groups of B-hPD-1/hCD40 mice that were injected with B16F10-hPD-L1 cells, and were treated with monospecific or bispecific antibodies. Physiological saline (PS) solution was injected as a control.

FIG. 13 shows average tumor volume on day 25 post grouping in different groups of B-hPD-1/hCD40 mice that were injected with MC38-hPD-L1 cells, and were treated with monospecific or bispecific antibodies. Physiological saline (PS) solution was injected as a control.

FIG. 14 is a graph showing body weight over time of B-hPD-1/hCD40 mice that were injected with MC38-hPD-L1 cells, and were treated with monospecific or bispecific antibodies. Physiological saline (PS) solution was injected as a control.

FIG. 15 is a graph showing percentage change of body weight over time of B-hPD-1/hCD40 mice that were injected with MC38-hPD-L1 cells, and were treated with monospecific or bispecific antibodies. Physiological saline (PS) solution was injected as a control.

FIG. 16 is a graph showing average tumor volume in different groups of B-hPD-1/hCD40 mice that were injected with MC38-hPD-L1 cells, and were treated with monospecific or bispecific antibodies. Physiological saline (PS) solution was injected as a control.

FIG. 17A shows mouse blood ALT level on day 7 post grouping in different groups of B-hPD-1/hCD40 mice that were injected with MC38-hPD-L1 cells, and were treated with monospecific or bispecific antibodies.

FIG. 17B shows mouse blood ALT level on day 13 post grouping in different groups of B-hPD-1/hCD40 mice that were injected with MC38-hPD-L1 cells, and were treated with monospecific or bispecific antibodies.

FIG. 17C shows mouse blood AST level on day 7 post grouping in different groups of B-hPD-1/hCD40 mice that were injected with MC38-hPD-L1 cells, and were treated with monospecific or bispecific antibodies.

FIG. 17D shows mouse blood AST level on day 13 post grouping in different groups of B-hPD-1/hCD40 mice that were injected with MC38-hPD-L1 cells, and were treated with monospecific or bispecific antibodies.

FIG. 17E shows a representative histological section image of mouse liver and kidney in G1 group B-hPD-1/hCD40 mice that were injected with MC38-hPD-L1 cells, and were treated with PBS.

FIG. 17F shows a representative histological section image of mouse liver and kidney in G2 group B-hPD-1/hCD40 mice that were injected with MC38-hPD-L1 cells, and were treated with Selicrelumab.

FIG. 17G shows a representative histological section image of mouse liver and kidney in G3 group B-hPD-1/hCD40 mice that were injected with MC38-hPD-L1 cells, and were treated with anti-CD40 monoclonal antibody 6A7-H4K2-IgG2.

FIG. 17H shows a representative histological section image of mouse liver and kidney in G4 group B-hPD-1/hCD40 mice that were injected with MC38-hPD-L1 cells, and were treated with anti-PD1 monoclonal antibody 1A7-H2K3-IgG4.

FIG. 17I shows a representative histological section image of mouse liver and kidney in G5 group B-hPD-1/hCD40 mice that were injected with MC38-hPD-L1 cells, and were treated with ScFV-HC-IgG4.

FIG. 17J shows a representative histological section image of mouse liver and kidney in G1 group B-hPD-1/hCD40 mice that were injected with MC38-hPD-L1 cells, and were treated with combination of anti-PD1 monoclonal antibody 1A7-H2K3-IgG4 and anti-CD40 monoclonal antibody 6A7-H4K2-IgG2 (Combo).

FIG. 18A is a schematic structure of bispecific antibody PD1-C40-6A7-FV3A.

FIG. 18B shows a gel electrophoresis result of PD1-C40-6A7-FV3A. M is marker. PD1-C40-6A7-FV3A is in lane 4.

FIG. 18C shows luminescence level in Jurkat-Luc-hCD40 cells in the presence of CHO-K1-hFcγRIIB cells and a selected antibody. The antibody was selected from anti-CD40 monoclonal antibody 6A7-H4K2-IgG2 (CD40), Fab-ScFv-IgG4, and PD1-C40-6A7-FV3A. The luminescence signal is expressed as relative light units (Rlu).

FIG. 18D shows luminescence level in Jurkat-Luc-hCD40 cells in the presence of CHO-K1-hPD-1 cells (CHO PD-1) and a selected antibody. The antibody was selected from a combination of anti-PD-1 monoclonal antibody 1A7-H2K3-IgG4 and anti-CD40 monoclonal antibody 6A7-H4K2-IgG2 (PD-1 +CD40), Fab-ScFv-IgG4, and PD1-C40-6A7-FV3A. The luminescence signal is expressed as relative light units (Rlu).

FIG. 19 lists CDR sequences of anti-PD1 antibodies 25-1A7, 18-3F1, and 3-6G1 as defined by Kabat numbering.

FIG. 20 lists CDR sequences of anti-PD1 antibodies 25-1A7, 18-3F1, and 3-6G1 as defined by Chothia numbering.

FIG. 21 lists amino acid sequences of human, mouse, and chimeric PD-1.

FIG. 22 lists amino acid sequences of heavy chain variable regions and light chain variable regions of humanized and mouse anti-PD1 antibodies.

FIG. 23 lists CDR sequences of anti-CD40 antibodies 03-7F10, 06-6A7, and 07-4H6 as defined by Kabat numbering.

FIG. 24 lists CDR sequences of anti-CD40 antibodies 03-7F10, 06-6A7, and 07-4H6 as defined by Chothia numbering.

FIG. 25 lists amino acid sequences of human, mouse, and chimeric CD40.

FIG. 26 lists amino acid sequences of heavy chain variable regions and light chain variable regions of humanized and mouse anti-CD40 antibodies.

FIG. 27 is a graph showing body weight over time of B-hPD-1/hCD40 mice that were injected with B16F10-hPD-L1 cells, and were treated with monospecific or bispecific antibodies. PBS solution was injected as a control.

FIG. 28 is a graph showing tumor volume over time of B-hPD-1/hCD40 mice that were injected with B16F10-hPD-L1 cells, and were treated with monospecific or bispecific antibodies. PBS solution was injected as a control.

FIG. 29A is a graph showing tumor volume of B-hPD-1/hCD40 mice that were injected with B16F10-hPD-L1 cells, and were treated with 0.1-30 mg/kg bispecific antibody ScFV-HC-IgG1-LALA (G2-G7) over a period of 70 days post grouping. PBS solution was injected as a control (G1).

FIG. 29B is a graph showing percentage of survived B-hPD-1/hCD40 mice that were injected with B16F10-hPD-L1 cells, and were treated with 0.1-30 mg/kg bispecific antibody ScFV-HC-IgG1-LALA (G2-G7) over a period of 70 days post grouping. PBS solution was injected as a control (G1).

FIG. 30A is a set of histograms showing percentage of mouse CD3+ T cells in CD45+ leukocytes (I); percentage of mouse CD8+ T cells in CD45+ leukocytes (II); and specific ratio of CD8+ T cells to Tregs in T cells (III) in a MC38 tumor model.

FIG. 30B is a set of histograms showing percentage of human PD-1 (CD279) positive cells in CD8+ T cells (I); percentage of human PD-1 positive cells in Tregs (II), percentage of human PD-1 positive cells in CD4+ (non-Treg) T cells (III), and percentage of human PD-1 positive cells in NK cells (IV) in a MC38 tumor model.

FIG. 30C is a set of histograms showing percentage of dendritic cells (DC) in mouse CD45+ leukocytes (I); percentage of myeloid-derived suppressor cells (MDSC) in mouse CD45+ leukocytes (II); percentage of CD80+ cells in DC cells (III); and percentage of MHCII+ cells in DC cells (IV) in a MC38 tumor model.

FIG. 30D is a set of histograms showing percentage of mouse CD3+ T cells in CD45+ leukocytes (I); percentage of mouse CD8+ T cells in CD45+ leukocytes (II); and specific ratio of CD8+ T cells to Tregs in T cells (III) in a B16F10 tumor model.

FIG. 30E is a set of histograms showing percentage of human PD-1 (CD279) positive cells in CD8+ T cells (I); percentage of human PD-1 positive cells in Tregs (II), percentage of human PD-1 positive cells in CD4+ (non-Treg) T cells (III), and percentage of human PD-1 positive cells in NK cells (IV) in a B 16F10 tumor model.

FIG. 30F is a set of histograms showing percentage of dendritic cells (DC) in mouse CD45+ leukocytes (I); percentage of myeloid-derived suppressor cells (MDSC) in mouse CD45+ leukocytes (II); percentage of CD80+ cells in DC cells (III); percentage of CD86+ cells in DC cells (IV); and percentage of MHCII+ cells in DC cells (V) in a B16F10 tumor model.

FIGS. 31A-31B show strategies of flow cytometry analysis.

FIG. 32 is a graph showing body weight over time of B-hPD-1/hCD40 mice that were injected with MC38-hPD-L1 cells, and were treated with monoclonal antibodies (G2-G4), bispecific antibodies (G5 and G6), or combination of monospecific antibodies (G7 and G8). PBS solution was injected as a control (G1).

FIG. 33 is a graph showing body weight change over time of B-hPD-1/hCD40 mice that were injected with MC38-hPD-L1 cells, and were treated with monoclonal antibodies (G2-G4), bispecific antibodies (G5 and G6), or combination of monospecific antibodies (G7 and G8). PBS solution was injected as a control (G1).

FIG. 34 is a graph showing tumor volume over time of B-hPD-1/hCD40 mice that were injected with MC38-hPD-L1 cells, and were treated with monoclonal antibodies (G2-G4), bispecific antibodies (G5 and G6), or combination of monospecific antibodies (G7 and G8). PBS solution was injected as a control (G1).

FIG. 35 is a graph showing body weight over time of B-hPD-1/hCD40 mice that were injected with monoclonal antibodies (G2 and G3), bispecific antibodies with different structures (G4-G7), or bispecific antibody ScFV-HC-IgG1-LALA (G8). PBS solution was injected as a control (G1).

FIG. 36 is a graph showing body weight change over time of B-hPD-1/hCD40 mice that were injected with monoclonal antibodies (G2 and G3), bispecific antibodies with different structures (G4-G7), or bispecific antibody ScFV-HC-IgG1-LALA (G8). PBS solution was injected as a control (G1).

FIG. 37 is a DART-Fc schematic structure of bispecific antibody 1A7-selicrelumab-DART-IgG4. DART refers to dual-affinity re-targeting antibody.

FIG. 38A shows mouse blood ALT level on Day 7 post grouping in different groups of B-hPD-1/hCD40 mice that were injected with PBS (G1), monoclonal antibodies (G2 and G3), bispecific antibodies with different structures (G4-G7), or bispecific antibody ScFV-HC-IgG1-LALA (G8).

FIG. 38B shows mouse blood AST level on Day 7 post grouping in different groups of B-hPD-1/hCD40 mice that were injected with PBS (G1), monoclonal antibodies (G2 and G3), bispecific antibodies with different structures (G4-G7), or bispecific antibody ScFV-HC-IgG1-LALA (G8).

FIG. 39 is a graph showing body weight over time of B-hPD-1/hCD40 mice that were injected with MC38-hPD-L1 cells, and were treated with monoclonal antibodies (G2 and G3), combination of monospecific antibodies (G4), or bispecific antibodies (G5-G6), or PBS solution was injected as a control (G1).

FIG. 40 is a graph showing body weight change over time of B-hPD-1/hCD40 mice that were injected with MC38-hPD-L1 cells, and were treated with monoclonal antibodies (G2 and G3), combination of monospecific antibodies (G4), or bispecific antibodies (G5-G6), or PBS solution was injected as a control (G1).

FIG. 41 is a graph showing tumor volume over time of B-hPD-1/hCD40 mice that were injected with MC38-hPD-L1 cells, and were treated with monoclonal antibodies (G2 and G3), combination of monospecific antibodies (G4), or bispecific antibodies (G5-G6), or PBS solution was injected as a control (G1).

FIG. 42A shows luminescence level in Jurkat-Luc-hCD40 cells in the presence of Juakat-PD1 cells and a selected antibody. The antibody was selected from BsAbs Pembrolizumab-seli-HC-IgG4, 1A7-selicrelumab-FV3A-IgG4, 1A7-selicrelumab-FVHC-IgG4, 1A7-selicrelumab-FVKH-IgG4, 1A7-selicrelumab-DART-IgG4, and anti-CD40 monoclonal antibody Selicrelumab-IgG2. The luminescence signal is expressed as relative light units (Rlu).

FIG. 42B shows luminescence level in Jurkat-Luc-hCD40 cells in the presence of Juakat-PD1 cells and a selected antibody. The antibody was selected from BsAbs Pembrolizumab-6A7-HC-IgG1-LALA, SdAb-6A7-FVHC-IgG1-LALA, Pembrolizumab-APX005M-FVHC-IgG4; anti-CD40 monoclonal antibody APX005M-IgG1-S267E and 6A7-H4K2-IgG2. The luminescence signal is expressed as relative light units (Rlu).

FIG. 42C shows luminescence level in Jurkat-Luc-hCD40 cells in the absence of Juakat-luc-PD1 cells and a selected antibody. The antibody was selected from BsAbs Pembrolizumab-self-HC-IgG4, 1A7-selicrelumab-FV3A-IgG4, 1A7-selicrelumab-FVHC-IgG4, 1A7-selicrelumab-FVKH-IgG4, 1A7-selicrelumab-DART-IgG4, and anti-CD40 monoclonal antibody Selicrelumab-IgG2. The luminescence signal is expressed as relative light units (Rlu).

FIG. 42D shows luminescence level in Jurkat-Luc-hCD40 cells in the absence of Juakat-luc-PD1 cells and a selected antibody. The antibody was selected from BsAbs Pembrolizumab-6A7-HC-IgG1-LALA, SdAb-6A7-FVHC-IgG1-LALA, Pembrolizumab-APX005M-FVHC-IgG4; anti-CD40 monoclonal antibody APX005M-IgG1-S267E and 6A7-H4K2-IgG2. The luminescence signal is expressed as relative light units (Rlu).

FIGS. 43A-43B show hypothetical mechanism of PD1/CD40 bispecific antibody-induced CD40 signaling pathway activation.

FIG. 44A is a graph showing body weight over time of B-hPD-1/hCD40 mice that were injected with MC38-hPD-L1 cells, and were treated with monoclonal antibodies (G2, G3 and G4), bispecific antibodies (G5-G7),or combination of monospecific antibodies (G8 and G9). PS solution was injected as a control (G1).

FIGS. 44B is a graph showing body weight change over time of B-hPD-1/hCD40 mice that were injected with MC38-hPD-L1 cells, and were treated with monoclonal antibodies (G2, G3 and G4), bispecific antibodies (G5-G7),or combination of monospecific antibodies (G8 and G9). PS solution was injected as a control (G1).

FIGS. 44C is a graph showing tumor volume over time of B-hPD-1/hPD-L1/hCD40 mice that were injected with MC38-hPD-L1 cells, and were treated with monoclonal antibodies (G2, G3 and G4), bispecific antibodies (G5-G7),or combination of monospecific antibodies (G8 and G9). PS solution was injected as a control (G1).

FIG. 45 is a graph showing body weight over time of B-hPD-1/hPD-L1/hCD40 mice that were injected with MC38-hPD-L1 cells, and were treated with ScFV-HC-IgG1-LALA (G2 and G5), Atezolizumab-6A7-FVHC-IgG1-LALA (G3 and G6), or Avelumab-6A7-FVHC-IgG1-LALA (G4 and G7). PBS solution was injected as a control (G1).

FIG. 46 is a graph showing body weight change over time of B-hPD-1/hPD-L1/hCD40 mice that were injected with MC38-hPD-L1 cells, and were treated with ScFV-HC-IgG1-LALA (G2 and G5), Atezolizumab-6A7-FVHC-IgG1-LALA (G3 and G6), or Avelumab-6A7-FVHC-IgG1-LALA (G4 and G7). PBS solution was injected as a control (G1).

FIG. 47 is a graph showing tumor volume over time of B-hPD-1/hPD-L1/hCD40 mice that were injected with MC38-hPD-L1 cells, and were treated with ScFV-HC-IgG1-LALA (G2 and G5), Atezolizumab-6A7-FVHC-IgG1-LALA (G3 and G6), or Avelumab-6A7-FVHC-IgG1-LALA (G4 and G7). PBS solution was injected as a control (G1).

FIG. 48 lists sequences described in the disclosure.

### DETAILED DESCRIPTION

A bispecific antibody or antigen-binding fragment thereof is an artificial protein that can simultaneously bind to two different epitopes (e.g., on two different antigens).

In some embodiments, a bispecific antibody or antigen-binding fragment thereof can be constructed by modifying an immunoglobulin, e.g., IgG. For example, the Fab region of an anti-PD-1 IgG can be replaced with an scFv domain targeting CD40. Alternatively, an scFv domain targeting CD40 can be linked to the C-terminus of an anti-PD-1 IgG heavy chain.

In some embodiments, a bispecific antibody or antigen-binding fragment thereof can have two arms (Arms A and B). Each arm has one heavy chain variable region and one light chain variable region, forming an antigen-binding domain (or an antigen binding site).

The bispecific antibody or antigen-binding fragment thereof can be IgG-like and non-IgG-like. The IgG-like bispecific antibody can have two Fab arms and one Fc region, and the two Fab arms bind to different antigens. The non-IgG-like bispecific antibody or antigen-binding fragment can be e.g., chemically linked Fabs (e.g., two Fab regions are chemically linked), and single-chain variable fragments (scFVs). For example, a scFV can have two heavy chain variable regions and two light chain variable regions. In some embodiments, one arm is a scFV polypeptide. In some embodiments, both arms are scFV polypeptides.

### Anti-PD-1/CD40 Antigen-Binding Protein Construct

The immune system can differentiate between normal cells in the body and those it sees as “foreign”, which allows the immune system to attack the foreign cells while leaving the normal cells alone. This mechanism sometimes involves proteins called immune checkpoints. Immune checkpoints are molecules in the immune system that either turn up a signal (co-stimulatory molecules) or turn down a signal.

Checkpoint inhibitors can prevent the immune system from attacking normal tissue and thereby preventing autoimmune diseases. Many tumor cells also express checkpoint inhibitors. These tumor cells escape immune surveillance by co-opting certain immune-checkpoint pathways, particularly in T cells that are specific for tumor antigens (Creelan, Benjamin C. “Update on immune checkpoint inhibitors in lung cancer.” Cancer Control 21.1 (2014): 80-89). Because many immune checkpoints are initiated by ligand-receptor interactions, they can be readily blocked by antibodies against the ligands and/or their receptors.

The present disclosure provides anti-PD1/CD40 antigen-binding protein constructs with various formats as described herein. Without wishing to be bound by theory, it is hypothesized in the presence of PD-1 expressing cells, a PD1/CD40 bispecific antibody can effectively activate CD40 signaling pathway, possibly via CD40 clustering at the contact surface of the PD-1 expressing cells and CD40 expressing cells. The bispecific antibody can facilitate the enrichment of CD40 and PD1 molecules at the contact surface. This enrichment further increases CD40 clustering, thereby activating CD40 pathway.

PD-1 (programmed death-1) is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 is mainly expressed on the surfaces of T cells and primary B cells; two ligands of PD-1 (PD-L1 and PD-L2) are widely expressed in antigen-presenting cells (APCs). The interaction of PD-1 with its ligands plays an important role in the negative regulation of the immune response. Inhibition the binding between PD-1 and its ligand can make the tumor cells exposed to the killing effect of the immune system, and thus can reach the effect of killing tumor tissues and treating cancers.

PD-L1 is expressed on the neoplastic cells of many different cancers. By binding to PD-1 on T-cells leading to its inhibition, PD-L1 expression is a major mechanism by which tumor cells can evade immune attack. PD-L1 over-expression may conceptually be due to 2 mechanisms, intrinsic and adaptive. Intrinsic expression of PD-L1 on cancer cells is related to cellular/genetic aberrations in these neoplastic cells. Activation of cellular signaling including the AKT and STAT pathways results in increased PD-L1 expression. In primary mediastinal B-cell lymphomas, gene fusion of the MHC class II transactivator (CIITA) with PD-L1 or PD-L2 occurs, resulting in over expression of these proteins. Amplification of chromosome 9p23-24, where PD-L1 and PD-L2 are located, leads to increased expression of both proteins in classical Hodgkin lymphoma. Adaptive mechanisms are related to induction of PD-L1 expression in the tumor microenvironment. PD-L1 can be induced on neoplastic cells in response to interferon γ. In microsatellite instability colon cancer, PD-L1 is mainly expressed on myeloid cells in the tumors, which then suppress cytotoxic T-cell function.

The use of PD-1 blockade to enhance anti-tumor immunity originated from observations in chronic infection models, where preventing PD-1 interactions reversed T-cell exhaustion. Similarly, blockade of PD-1 prevents T-cell PD-1/tumor cell PD-L1 or T-cell PD-1/tumor cell PD-L2 interaction, leading to restoration of T-cell mediated anti-tumor immunity.

A detailed description of PD-1, and the use of anti-PD-1 antibodies to treat cancers are described, e.g., in Topalian, Suzanne L., et al. “Safety, activity, and immune correlates of anti-PD-1 antibody in cancer.” New England Journal of Medicine 366.26 (2012): 2443-2454; Hirano, Fumiya, et al. “Blockade of B7-H1 and PD-1 by monoclonal antibodies potentiates cancer therapeutic immunity.” Cancer research 65.3 (2005): 1089-1096; Raedler, Lisa A. “Keytruda (pembrolizumab): first PD-1 inhibitor approved for previously treated unresectable or metastatic melanoma.” American health & drug benefits 8.Spec Feature (2015): 96; Kwok, Gerry, et al. “Pembrolizumab (Keytruda).” (2016): 2777-2789; US 20170247454; U.S. Pat. Nos. 9,834,606 B; and 8,728,474; each of which is incorporated by reference in its entirety.

### CD40

CD40 (also known as Tumor Necrosis Factor Receptor Superfamily Member 5 or TNFRSF5) is a tumor necrosis factor receptor superfamily member expressed on antigen presenting cells (APC) such as dendritic cells (DC), macrophages, B cells, and monocytes as well as many non-immune cells and a wide range of tumors. Interaction with its trimeric ligand CD154 (also known as CD40 ligand or CD40L) on activated T helper cells results in APC activation, leading to the induction of adaptive immunity.

Physiologically, signaling via CD40 on APC is thought to represent a major component of T cell help and mediates in large part the capacity of helper T cells to license APC. Ligation of CD40 on DC, for example, induces increased surface expression of costimulatory and MHC molecules, production of proinflammatory cytokines, and enhanced T cell triggering. CD40 ligation on resting B cells increases antigen-presenting function and proliferation.

In pre-clinical models, rat anti-mouse CD40 mAb show remarkable therapeutic activity in the treatment of CD40+ B-cell lymphomas (with 80-100% of mice cured and immune to re-challenge in a CD8 T-cell dependent manner) and are also effective in various CD40-negative tumors. These mAb are able to clear bulk tumors from mice with near terminal disease. CD40 mAb have been investigated in clinical trials and are used for treating melanoma, pancreatic carcinoma, mesothelioma, hematological malignancies, especially Non-Hodgkin's lymphoma, lymphoma, chronic lymphocytic leukemia, and advanced solid tumors.

Therapeutic anti-CD40 antibodies show diverse activities ranging from strong agonism to antagonism. Currently there is no satisfactory explanation for this heterogeneity. The primary mechanistic rationale invoked for agonistic CD40 mAb is to activate host APC in order to induce clinically meaningful anti-tumor T-cell responses in patients. These include T cell-independent but macrophage-dependent triggering of tumor regression. CD40-activated macrophages can become tumoricidal, and least in pancreatic cancer, may also facilitate the depletion of tumor stroma which induces tumor collapse in vivo. Importantly, these mechanisms do not require expression of CD40 by the tumor, which has justified inclusion of patients with a broad range of tumors in many of the clinical trials. Insofar as these strategies aim to activate DC, macrophages, or both, the goal is not necessarily for the CD40 mAb to kill the cell it binds to, for example, via complement mediated cytotoxicity (CDC) or antibody dependent cellular cytoxicity (ADCC). Thus, by design, the strong agonistic antibody does not mediate CDC or ADCC.

In contrast, other human CD40 mAb can mediate CDC and ADCC against CD40+ tumors, such as nearly all B cell malignancies, a fraction of melanomas, and certain carcinomas. Finally, there is some evidence that ligation of CD40 on tumor cells promotes apoptosis and that this can be accomplished without engaging any immune effector pathway. This has been shown for CD40+ B cell malignancies and certain solid tumors such as CD40+ carcinomas and melanomas.

A detailed description of CD40 and its function can be found, e.g., in Vonderheide et al., “Agonistic CD40 antibodies and cancer therapy.” (2013): 1035-1043; Beatty, et al. “CD40 agonists alter tumor stroma and show efficacy against pancreatic carcinoma in mice and humans.” Science 331.6024 (2011): 1612-1616; Vonderheide, et al. “Clinical activity and immune modulation in cancer patients treated with CP-870,893, a novel CD40 agonist monoclonal antibody.” Journal of Clinical Oncology 25.7 (2007): 876-883; each of which is incorporated by reference in its entirety.

In some embodiments, the disclosure provides antigen-binding constructs (e.g., bispecific antibodies) that specifically bind to two difference antigens (e.g., PD-1 and CD40). In some embodiments, the antigen-binding constructs (e.g., bispecific antibodies) can bridge PD-1 expressing cells (e.g., T cells) and CD40-expressing cells (e.g., APC cells), thereby facilitating antigen-presenting, CD40 ligation, and/or T cell activation. In some embodiments, the antigen-binding constructs can block PD-1/PD-L1 pathway thereby activating the immune response. In some embodiments, the antigen-binding constructs (e.g., bispecific antibodies) can activate CD40 pathway in antigen presenting cells (APC) cells (e.g., dendritic cells or microphage), thereby induce anti-tumor responses. In some embodiments, the activation of CD40 induced by the antigen-binding constructs (e.g., bispecific antibodies) can occur only when cells expressing PD-1 are present (e.g., through bridging effects). Therefore, the antigen-binding constructs (e.g., bispecific antibodies) can induce immune responses within the local tumor microenvironment without causing immune activation in the whole body, which can induce side effects, e.g., toxicity in the liver. Furthermore, in some embodiments, the antigen-binding constructs (e.g., bispecific antibodies) cannot activate CD40 pathway via Fc receptor (e.g., FCγRIIB)-mediated CD40 clustering. In some embodiments, the methods as described herein further reduce toxicity in high FCγRIIB expression tissues, e.g., liver and/or kidney.

In addition, lymph nodes that lie immediately downstream of tumors (tumor-draining lymph nodes (TDLNs)) can undergo profound alterations due to the presence of the upstream tumor. The tumor-draining lymph nodes (TDLNs) are enriched for tumor-specific PD-1+ T cells which closely associate with PD-L1+ conventional dendritic cells. TDLN-targeted PD-1 pathway blockade can induce enhanced anti-tumor T cell immunity by seeding the tumor site with progenitor-exhausted T cells, resulting in improved tumor control. In the TDLN, the number and suppressor activity of regulatory T cells (Tregs) are also increased. Some of these Tregs may be generated de novo against specific tumor-derived antigens. In some cases, the presentation of new antigens in TDLNs not only fails to elicit a protective immune response but also actively creates systemic tolerance. Therefore, in one aspect, the antigen-binding constructs (e.g., bispecific antibodies) can also induce immune responses in tumor-draining lymph nodes, inhibit Tregs activities in tumor-draining lymph nodes, and suppress systemic tolerance for tumor antigens.

In some embodiments, the disclosure provides methods to reduce toxicity of an anti-CD40 monoclonal antibody (e.g., Selicrelumab). The methods include making a multispecific (e.g., bispecific) antibody containing the antigen-binding fragment of the anti-CD40 monoclonal antibody. In some embodiments, the multispecific antibody has an antigen-binding fragment for PD-1. In some embodiments, the multispecific (e.g., bispecific) antibody has a structure as described herein. In some embodiments, the toxicity is evaluated by blood biochemical tests (e.g., by measuring ALT and/or AST levels), or histopathological examination of liver. In some embodiments, the methods as described herein can reduce the toxicity of an anti-CD40 monoclonal antibody by to less than 80%, less than 70%, less than 60%, less than 50%, less than 40%, less than 30%, less than 20%, or less than 10% as compared to that of the unmodified anti-CD40 monoclonal antibody.

In some embodiments, the antibody or antigen-binding fragment thereof comprises CH1, CH2, and/or CH3 domains (e.g., CH2 and CH3 domains) of IgG4. In some embodiments, the antibody or antigen-binding fragment thereof comprises knob-into-hole (KIH) mutations. In some embodiments, the antibody or antigen-binding fragment thereof comprises CH1, CH2, CH3 domain of IgG4 in a first polypeptide chain, and CH2, CH3 domain of IgG4 in a second polypeptide chain. In some embodiments, the antibody or antigen-binding fragment thereof comprises an amino acid sequence that is at least 80%, 85%, 90%, or 95% identical to SEQ ID NO: 149, 150, or 151.

In some embodiments, the antibody or antigen-binding fragment thereof comprises CH1, CH2, and/or CH3 domains (e.g., CH1, CH2, and CH3 domains) of IgG1. In some embodiments, the antibody or antigen-binding fragment thereof comprises LALA mutations. In some embodiments, the antibody or antigen-binding fragment thereof comprises CH1, CH2, CH3 domain of IgG1 in a first polypeptide chain, and CH1, CH2, CH3 domain of IgG1 in a second polypeptide chain. In some embodiments, the antibody or antigen-binding fragment thereof comprises an amino acid sequence that is at least 80%, 85%, 90%, or 95% identical to SEQ ID NO: 147, or 148.

In some embodiments, the antibody or antigen-binding fragment thereof comprises an amino acid sequence lacking the lysine residue at the C-terminus of IgG (e.g., IgG1 or IgG4) heavy chain constant domain 3 (CH3). In some embodiments, the lack of C-terminus lysine residue of IgG CH3 domain decreases degradation level of the antibody or antigen-binding fragment thereof, e.g., by at least or about 10%, 20%, 30%, 40%, or 50% as compared to an antibody or antigen-binding fragment comprising the C-terminus lysine residue of IgG CH3.

In some embodiments, the antibody or antigen-binding fragment thereof comprises a light chain constant domain (CL). In some embodiments, the CL comprises an amino acid sequence that is at least 80%, 85%, 90%, or 95% identical to SEQ ID NO: 146.

In one aspect, the disclosure provides a bispecific antibody or antigen-binding fragment thereof, comprising

(a) a heavy chain polypeptide, comprising preferably from N-terminus to C-terminus: a first heavy chain variable region (VH1), a heavy chain constant region 1 (CH1), a heavy chain constant region 2 (CH2), and a heavy chain constant region 3 (CH3);

(b) a light chain polypeptide, comprising preferably from N-terminus to C-terminus: a first light chain variable region (VL1), and a light chain constant region (CL); and (c) a single-chain variable fragment polypeptide, comprising preferably from N-terminus to C-terminus: an scFv domain, a heavy chain constant region 2 (CH2), and a heavy chain constant region 3 (CH3).

In some embodiments, the scFv domain comprises from N-terminus to C-terminus: a second heavy chain variable region, a linker peptide sequence, a second light chain variable region.

In some embodiments, the scFv domain comprises from N-terminus to C-terminus: a second light chain variable region, a linker peptide sequence, a second heavy chain variable region.

In some embodiments, the first heavy chain variable region and the first light chain variable region associate with each other, forming a first antigen binding site that specifically binds to PD-1. In some embodiments, the second heavy chain variable region and the second light chain variable region associate with each other, forming a second antigen binding site that specifically binds to CD40.

In some embodiments, the second heavy chain variable region, the linker peptide sequence, and the second light chain variable region together forms an scFv domain that specifically binds to CD40. In some embodiments, the linker peptide sequence comprises a sequence that is at least 80%, 85%, 90%, 95%, or 100% identical to SEQ ID NO: 152, 153 or 154. In some embodiments, the CH1, CH2, CH3, and/or CL domains are from human IgG (e.g., IgG1 or IgG4). In some embodiments, the human IgG (e.g., IgG1 or IgG4) comprises KIH and/or LALA mutations.

In some embodiments, sequence of the heavy chain polypeptide is set forth in SEQ ID NO: 130. In some embodiments, sequence of the light chain polypeptide is set forth in SEQ ID NO: 131. In some embodiments, sequence of the single-chain variable fragment polypeptide is set forth in SEQ ID NO: 132 or 133 (e.g., SEQ ID NO: 132). In some embodiments, the sequences of the PD-1 heavy chain and light chain of Fab-ScFV-IgG4 are set forth in SEQ ID NO: 130 and SEQ ID NO: 131, respectively; and the sequence of the CD40 arm of Fab-scFv-IgG4 is set forth in SEQ ID NO: 132.

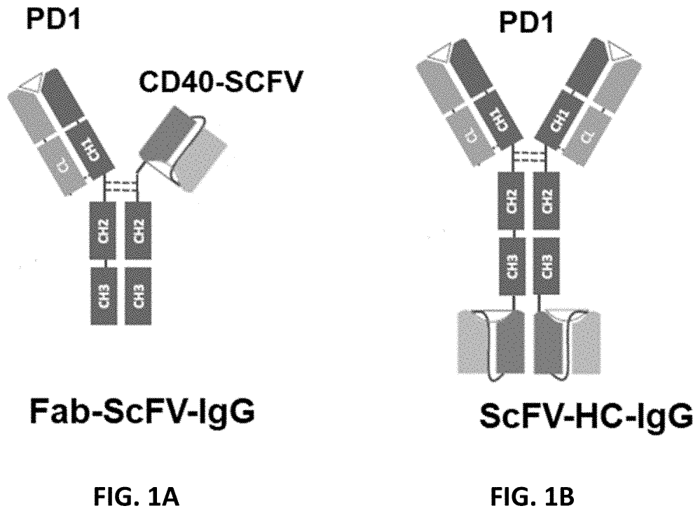

In some embodiments, the bispecific antibody or antigen-binding fragment thereof described herein has a schematic structure shown in FIG. 1A. In some embodiments, the sequence of the heavy chain polypeptide is at least 80%, 85%, 90%, 95%, or 100% identical to SEQ ID NO: 166. In some embodiments, the sequence of the light chain polypeptide is at least 80%, 85%, 90%, 95%, or 100% identical SEQ ID NO: 167. In some embodiments, the sequence of the single-chain variable fragment peptide is at least 80%, 85%, 90%, 95%, or 100% identical SEQ ID NO: 206. In some embodiments, the heavy chain sequence and the light chain sequence of the PD-1 arm of the bispecific antibody or antigen-binding fragment thereof described herein are set forth in SEQ ID NO: 166 and SEQ ID NO: 167, respectively; and the sequence of the CD40 arm of the bispecific antibody or antigen-binding fragment thereof described herein is set forth in SEQ ID NO: 173.

In one aspect, the disclosure provides a bispecific antibody or antigen-binding fragment thereof, comprising

(a) a first heavy chain polypeptide comprising, preferably from N-terminus to C-terminus: a first heavy chain variable region (VH1), a heavy chain constant region 1 (CH1), a heavy chain constant region 2 (CH2), a heavy chain constant region 3 (CH3), a first linker peptide sequence, and an scFv domain;

(b) a first light chain polypeptide comprising, preferably from N-terminus to C-terminus: a first light chain variable region (VL1), and a first light chain constant region (CL).

In some embodiments, the scFv domain comprises from N-terminus to C-terminus: a second light chain variable region (VL2), a second linker peptide sequence, and a second heavy chain variable region (VH2).

In some embodiments, the scFv domain comprises from N-terminus to C-terminus: a second heavy chain variable region (VH2), a second linker peptide sequence, and a second light chain variable region (VL2).

In some embodiments, the first antigen binding site specifically binds to an immune checkpoint molecule. In some embodiments, the first antigen binding site that specifically binds to programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), Glucocorticoid-Induced TNFR-Related Protein (GITR), T-cell immunoglobulin and mucin-domain containing-3 (TIM-3), or TNF Receptor Superfamily Member 4 (TNFRSF4 or OX40). In some embodiments, the first antigen binding site specifically binds to PD-1.

In some embodiments, the first antigen binding site that specifically binds to a cancer specific antigen or a cancer-associated antigen. As used herein, the term “cancer specific antigen” refers to antigens that are specifically expressed on cancer cell surfaces. These antigens can be used to identify tumor cells. Normal cells rarely express cancer specific antigens. Some exemplary cancer specific antigens include, e.g., CD20, PSA, PSCA, PD-L1, Her2, Her3, Her1, β-Catenin, CD19, CEACAM3, EGFR, c-Met, EPCAM, PSMA, CD40, MUC1, and IGF1R, etc. PSA are primarily expressed on prostate cancer cells, and Her2 are primarily expressed on breast cancer cells. As used herein, the term “cancer-associated antigen” refers to antigens that are expressed at a relatively high level on cancer cells but may be also expressed at a relatively low level on normal cells. CD55, CD59, CD46 and many adhesion molecules such as N-cadherin, VE-cadherin, NCAM, Mel-CAM, ICAM, NrCAM, VCAM1, ALCAM, MCAM, etc., are cancer-associated antigens. While both cancer specific antigen and cancer-associated antigen are expressed on cancer cell surface, the difference between a cancer specific antigen and a cancer-associated antigen is that the cancer-associated antigen is also expressed on normal cells, but at a relative low level as compared to the level on cancer cells. In contrast, a cancer specific antigen is rarely expressed on normal cells, and even if it is expressed on normal cells, the amount is extremely low.

In some embodiments, the second heavy chain variable region and the second light chain variable region associate with each other, forming a second antigen binding site that specifically binds to CD40. In some embodiments, the second antigen binding site comprises or consists of a ScFv or a VHH.

In some embodiments, the second antigen binding site is linked to the Fc region of an antibody (e.g., IgG). In some embodiments, the second antigen binding site is linked to the C-terminal of the Fc region. In some embodiments, the second antigen binding site is inserted to the Fc region, e.g., at a 3A site.

The 3A site refers to a region in the Fc, wherein a non-native peptide can be inserted without interfering the function of the immunoglobulins or the biological activity of the fused polypeptide. The 3A site starts from position 344 to position 382 (EU numbering). In some embodiments, the non-native polypeptide replaces 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 amino acids at the fusion site or is inserted between any of the two amino acids at this fusion site. In some embodiments, the fusion site is located at a region from position 351 to 362 (EU numbering). In some embodiments, the non-native polypeptide replaces 1, 2, 3, 4, 5, 6, 7, 8, 9, 10, 11, 12, or all amino acids (e.g., 351-362) at the fusion site, or is inserted between any of the two amino acids at this fusion site, e.g., inserted at the position 351-352, 352-353, 353-354, 354-355, 355-356, 356-357, 357-358, 358-359, 359-360, 360-361, or 361-362. In some embodiments, the two residues are selected from any two of positions 350, 351, 352, 353, 354, 355, 356, 357, 358, 359, 360, 361, 362, and 363 of the heavy chain CH3 domain according to EU numbering. In some embodiments, the non-native polypeptide replaces amino acids 358-362 at the fusion site.

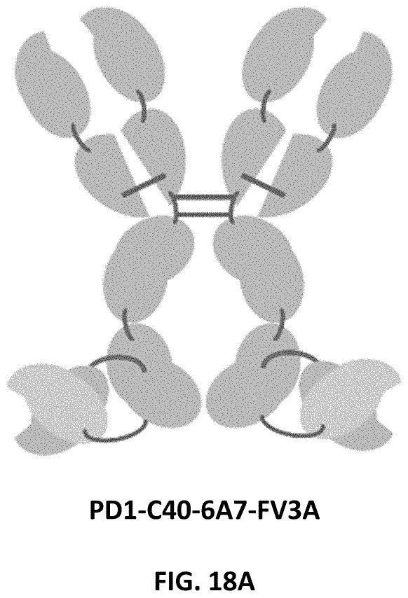

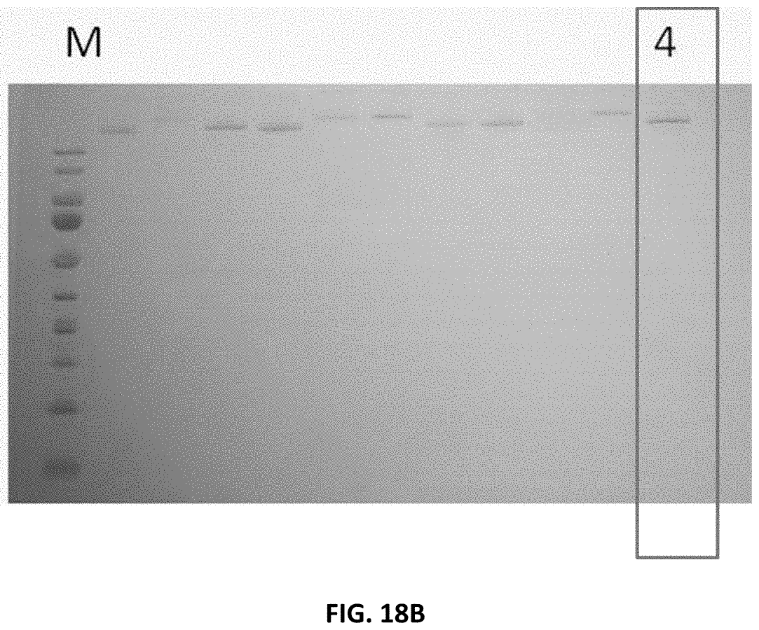

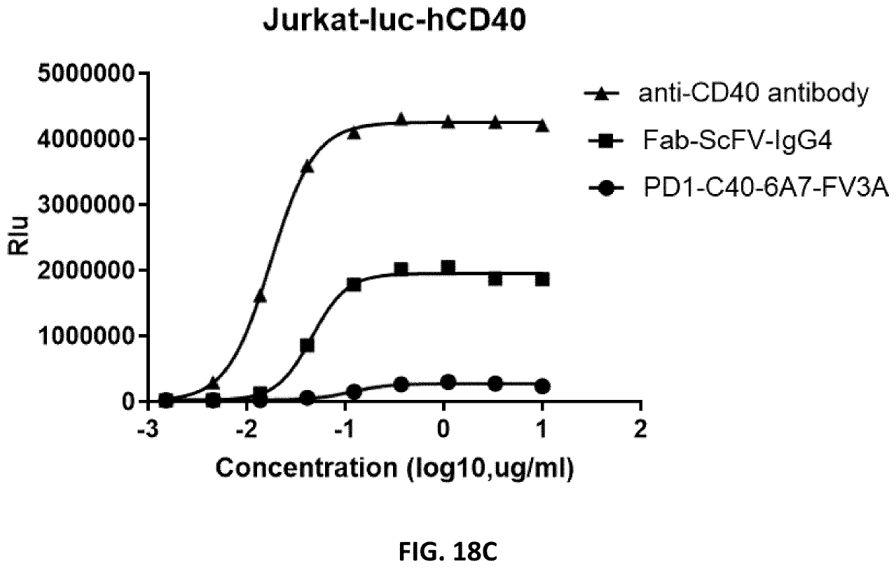

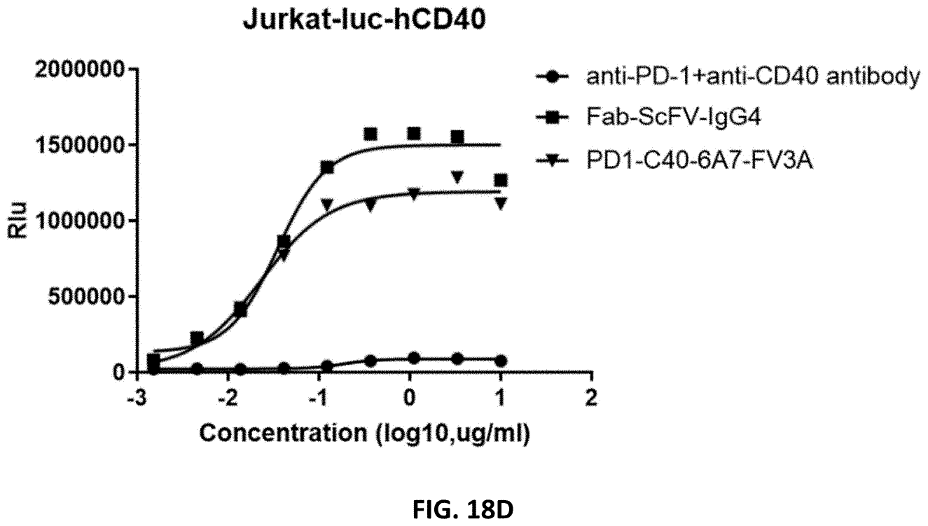

In some embodiments, an anti-CD40 scFv is fused to each of the heavy chain CH3 domain of an anti-PD-1 monoclonal antibody. In some embodiments, the bispecific antibody or antigen-binding fragment thereof described herein has a schematic structure shown in FIG. 18A. In some embodiments, the fused heavy chain polypeptide has a sequence that is at least 80%, 85%, 90%, 95%, or 100% identical to SEQ ID NO: 161. In some embodiments, the light chain polypeptide has a sequence that is at least 80%, 85%, 90%, 95%, or 100% identical to SEQ ID NO: 141. In some embodiments, the fused heavy chain polypeptide comprises an anti-PD-1 heavy chain variable region as described herein. In some embodiments, the light chain polypeptide comprises an anti-PD-1 light chain variable region as described herein.

In some embodiments, the fused heavy chain polypeptide has a sequence that is at least 80%, 85%, 90%, 95%, or 100% identical to SEQ ID NO: 168. In some embodiments, the light chain polypeptide has a sequence that is at least 80%, 85%, 90%, 95%, or 100% identical to SEQ ID NO: 169.

In some embodiments, the bispecific antibody or antigen-binding fragment thereof further comprises: a second heavy chain polypeptide that is at least 90%, 95%, or 100% identical to the first heavy chain polypeptide; and a second light chain polypeptide that is at least 90%, 95%, or 100% identical to the first light chain polypeptide.

In some embodiments, the second light chain variable region, the linker peptide sequence, and the second heavy chain variable region together forms an scFv domain that specifically binds to CD40. In some embodiments, the first linker peptide sequence comprises a sequence that is at least 80%, 85%, 90%, 95%, or 100% identical to SEQ ID NO: 153 or 154. In some embodiments, the second linker peptide sequence comprises a sequence that is at least 80%, 85%, 90%, 95%, or 100% identical to SEQ ID NO: 153 or 154. In some embodiments, the CH1, CH2, CH3, and/or CL domains are from human IgG (e.g., IgG1 or IgG4). In some embodiments, the human IgG (e.g., IgG1 or IgG4) comprises KIH and/or LALA mutations.

In some embodiments, the antibody or antigen-binding fragment thereof described herein can block the PD-1/PD-L1 pathway.

In some embodiments, the bispecific antibody or antigen-binding fragment thereof described herein has a schematic structure shown in FIG. 1B. In some embodiments, sequence of the first heavy chain polypeptide is set forth in SEQ ID NO: 134 or 135. In some embodiments, sequence of the first light chain polypeptide is set forth in SEQ ID NO: 136. In some embodiments, sequence of the first heavy chain polypeptide is set forth in SEQ ID NO: 137 or 138. In some embodiments, sequence of the first light chain polypeptide is set forth in SEQ ID NO: 139. In some embodiments, the sequence of the modified heavy chain of ScFv-HC-IgG4 is set forth in SEQ ID NO: 134, and the sequence of the light chain of ScFv-HC-IgG4 is set forth in SEQ ID NO: 136. In some embodiments, the sequence of the modified heavy chain of ScFv-HC-IgG1-LALA is set forth in SEQ ID NO: 137, and the sequence of the light chain of ScFv-HC-IgG1-LALA is set forth in SEQ ID NO: 139.

In some embodiments, the sequence of the first heavy chain polypeptide comprises a sequence that is at least 80%, 85%, 90%, 95%, or 100% identical to SEQ ID NO: 162, and the sequence of the first light chain polypeptide comprises a sequence that is at least 80%, 85%, 90%, 95%, or 100% identical to SEQ ID NO: 163. In some embodiments, the sequence of the first heavy chain polypeptide comprises a sequence that is at least 80%, 85%, 90%, 95%, or 100% identical to SEQ ID NO: 164, and the sequence of the first light chain polypeptide comprises a sequence that is at least 80%, 85%, 90%, 95%, or 100% identical to SEQ ID NO: 165. In some embodiments, the sequence of the first heavy chain polypeptide comprises a sequence that is at least 80%, 85%, 90%, 95%, or 100% identical to SEQ ID NO: 175, and the sequence of the first light chain polypeptide comprises a sequence that is at least 80%, 85%, 90%, 95%, or 100% identical to SEQ ID NO: 176. In some embodiments, the sequence of the first heavy chain polypeptide comprises a sequence that is at least 80%, 85%, 90%, 95%, or 100% identical to SEQ ID NO: 177, and the sequence of the first light chain polypeptide comprises a sequence that is at least 80%, 85%, 90%, 95%, or 100% identical to SEQ ID NO: 178.

In some embodiments, the sequence of the first heavy chain polypeptide comprises a sequence that is at least 80%, 85%, 90%, 95%, or 100% identical to SEQ ID NO: 172, and the sequence of the first light chain polypeptide comprises a sequence that is at least 80%, 85%, 90%, 95%, or 100% identical to SEQ ID NO: 174. In some embodiments, the disclosure provides a bispecific antibody or antigen-binding fragment thereof, comprising a polypeptide chain comprising, preferably from N-terminus to C-terminus: a single variable domain (VHH) that binds to PD-1, an optional CH1, a CH2, a CH3, a linker peptide sequence, and a scFv domain. In some embodiments, the VHH comprises a sequence that is at least 80%, 85%, 90%, 95%, or 100% identical to SEQ ID NO: 204.

In some embodiments, the variable regions (e.g., VH and/or VL) of a selected anti-PD-1 antibody are used to replace the corresponding anti-PD-1 variable regions (e.g., VH and/or VL) of a bispecific antibody as described herein according to their sequences. For example, sequence of the VH of the selected anti-PD-1 antibody can be selected from SEQ ID NO: 179, 181, 183, 185, 187, 189, 207, or 209; sequence of the VL of the selected anti-PD-1 antibody can be selected from SEQ ID NO: 180, 182, 184, 186, 188, 190, 208 or 210.

In some embodiments, both VH and VL of a selected anti-PD-1 antibody are used to replace the corresponding VH and VL of a bispecific antibody as described herein according to their sequences. For example, the VH of the selected anti-PD-1 antibody is SEQ ID NO: 179 and the VL of the selected anti-PD-1 antibody is SEQ ID NO: 180; the VH of the selected anti-PD-1 antibody is SEQ ID NO: 181 and the VL of the selected anti-PD-1 antibody is SEQ ID NO: 182; the VH of the selected anti-PD-1 antibody is SEQ ID NO: 183 and the VL of the selected anti-PD-1 antibody is SEQ ID NO: 184; the VH of the selected anti-PD-1 antibody is SEQ ID NO: 185 and the VL of the selected anti-PD-1 antibody is SEQ ID NO: 186; the VH of the selected anti-PD-1 antibody is SEQ ID NO: 187 and the VL of the selected anti-PD-1 antibody is SEQ ID NO: 188; the VH of the selected anti-PD-1 antibody is SEQ ID NO: 189 and the VL of the selected anti-PD-1 antibody is SEQ ID NO: 190; the VH of the selected anti-PD-1 antibody is SEQ ID NO: 207 and the VL of the selected anti-PD-1 antibody is SEQ ID NO: 208; the VH of the selected anti-PD-1 antibody is SEQ ID NO: 209 and the VL of the selected anti-PD-1 antibody is SEQ ID NO: 210.

In some embodiments, the variable regions (e.g., VH and/or VL) of a selected anti-CD40 antibody are used to replace the corresponding anti-CD40 variable regions (e.g., VH and/or VL) of a bispecific antibody as described herein according to their sequences. For example, sequence of the VH of the selected anti-PD-1 antibody can be selected from SEQ ID NO: 191, 193, 195, 197, or 199; sequence of the VL of the selected anti-PD-1 antibody can be selected from SEQ ID NO: 192, 194, 196, 198, or 200

In some embodiments, both VH and VL of a selected anti-CD40 antibody are used to replace the corresponding VH and VL of a bispecific antibody as described herein according to their sequences. For example, the VH of the selected anti-CD40 antibody is SEQ ID NO: 191 and the VL of the selected anti-CD40 antibody is SEQ ID NO: 192; the VH of the selected anti-CD40 antibody is SEQ ID NO: 193 and the VL of the selected anti-CD40 antibody is SEQ ID NO: 194; the VH of the selected anti-CD40 antibody is SEQ ID NO: 195 and the VL of the selected anti-CD40 antibody is SEQ ID NO: 196; the VH of the selected anti-CD40 antibody is SEQ ID NO: 197 and the VL of the selected anti-CD40 antibody is SEQ ID NO: 198; the VH of the selected anti-CD40 antibody is SEQ ID NO: 199 and the VL of the selected anti-CD40 antibody is SEQ ID NO: 200.

### Anti-CD40 Antibodies and Antigen-Binding Fragments

The disclosure provides several antibodies and antigen-binding fragments thereof that specifically bind to CD40. The anti-PD-1/CD40 antigen-binding protein constructs (e.g., bispecific antibodies) or various antigen-binding protein constructs can include an antigen binding site that is derived from these antibodies.

The antibodies and antigen-binding fragments described herein are capable of binding to CD40. The disclosure provides e.g., mouse anti-CD40 antibodies 03-7F10 (“7F10”), 06-6A7 (“6A7”), and 07-4H6 (“4H6”), and chimeric antibodies, the humanized antibodies thereof.

The CDR sequences for 7F10, and 7F10 derived antibodies (e.g., humanized antibodies) include CDRs of the heavy chain variable domain, SEQ ID NOs: 59-61, and CDRs of the light chain variable domain, SEQ ID NOs: 62-64 as defined by Kabat numbering. The CDRs can also be defined by Chothia system. Under the Chothia numbering, the CDR sequences of the heavy chain variable domain are set forth in SEQ ID NOs: 77-79, and CDR sequences of the light chain variable domain are set forth in SEQ ID NOs: 80-82.

Similarly, the CDR sequences for 6A7, and 6A7 derived antibodies include CDRs of the heavy chain variable domain, SEQ ID NOs: 65-67, and CDRs of the light chain variable domain, SEQ ID NOs: 68-70, as defined by Kabat numbering. Under Chothia numbering, the CDR sequences of the heavy chain variable domain are set forth in SEQ ID NOs: 83-85, and CDRs of the light chain variable domain are set forth in SEQ ID NOs: 86-88.

The CDR sequences for 4H6, and 4H6 derived antibodies include CDRs of the heavy chain variable domain, SEQ ID NOs: 71-73, and CDRs of the light chain variable domain, SEQ ID NOs: 74-76, as defined by Kabat numbering. Under Chothia numbering, the CDR sequences of the heavy chain variable domain are set forth in SEQ ID NOs: 89-91, and CDRs of the light chain variable domain are set forth in SEQ ID NOs: 92-94.

The amino acid sequence for heavy chain variable region and light variable region of humanized antibodies are also provided. As there are different ways to humanize the mouse antibody (e.g., sequence can be substituted by different amino acids), the heavy chain and the light chain of an antibody can have more than one versions of humanized sequences. The amino acid sequences for the heavy chain variable region of humanized 7F10 antibody are set forth in SEQ ID NOs: 98-100. The amino acid sequences for the light chain variable region of humanized 7F10 antibody are set forth in SEQ ID NOs: 101-104. Any of these heavy chain variable region sequences (SEQ ID NOs: 98-100) can be paired with any of these light chain variable region sequences (SEQ ID NOs: 101-104).

Similarly, the amino acid sequences for the heavy chain variable region of humanized 6A7 antibody are set forth in SEQ ID NOs: 105-108. The amino acid sequences for the light chain variable region of humanized 6A7 antibody are set forth in SEQ ID NOs: 109-111. The heavy chain variable region and the light chain variable region can also be modified to increase the stability or interaction. 6A7-H4 can be further modified to obtain SEQ ID NO: 126. 6A7-K2 can be further modified to obtain SEQ ID NO: 127. Similarly, the heavy chain variable region of mouse 6A7 antibody can be further modified to obtain SEQ ID NO: 128. The light chain variable region of mouse 6A7 antibody can be further modified to obtain SEQ ID NO: 129. Any of these heavy chain variable region sequences (SEQ ID NOs: 105-108, 126 and 128) can be paired with any of these light chain variable region sequences (SEQ ID NOs: 109-111, 127, and 129). The amino acid sequences for the heavy chain variable region of humanized 4H6 antibody are set forth in SEQ ID NOs: 112-115. The amino acid sequences for the light chain variable region of humanized 4H6 antibody are set forth in SEQ ID NOs: 116-119. Any of these heavy chain variable region sequences (SEQ ID NOs: 112-115) can be paired with any of these light chain variable region sequences (SEQ ID NOs: 116-119).

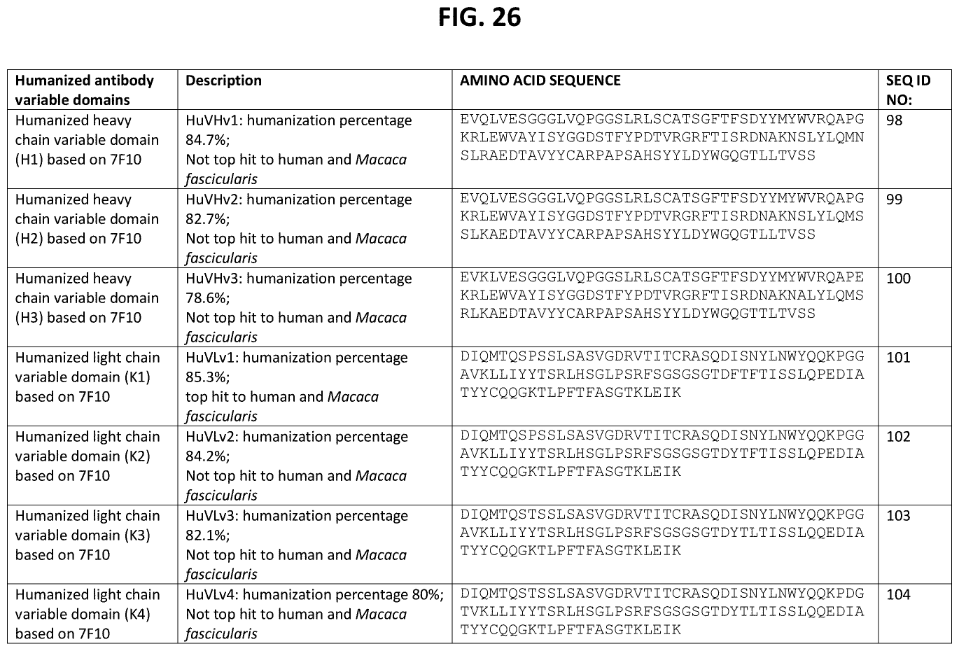

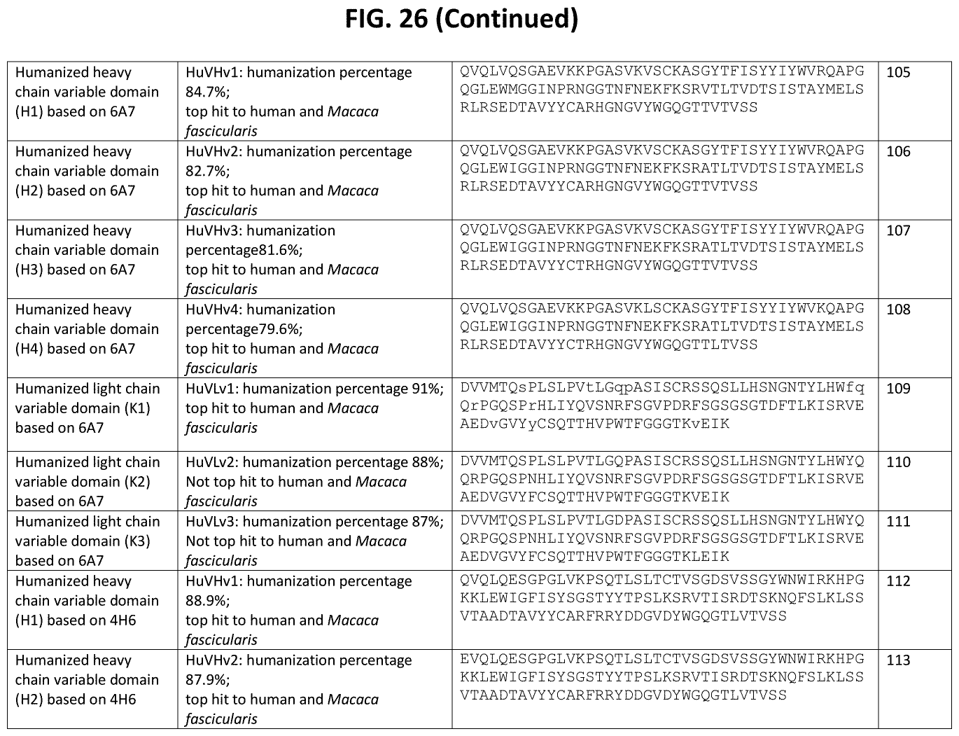

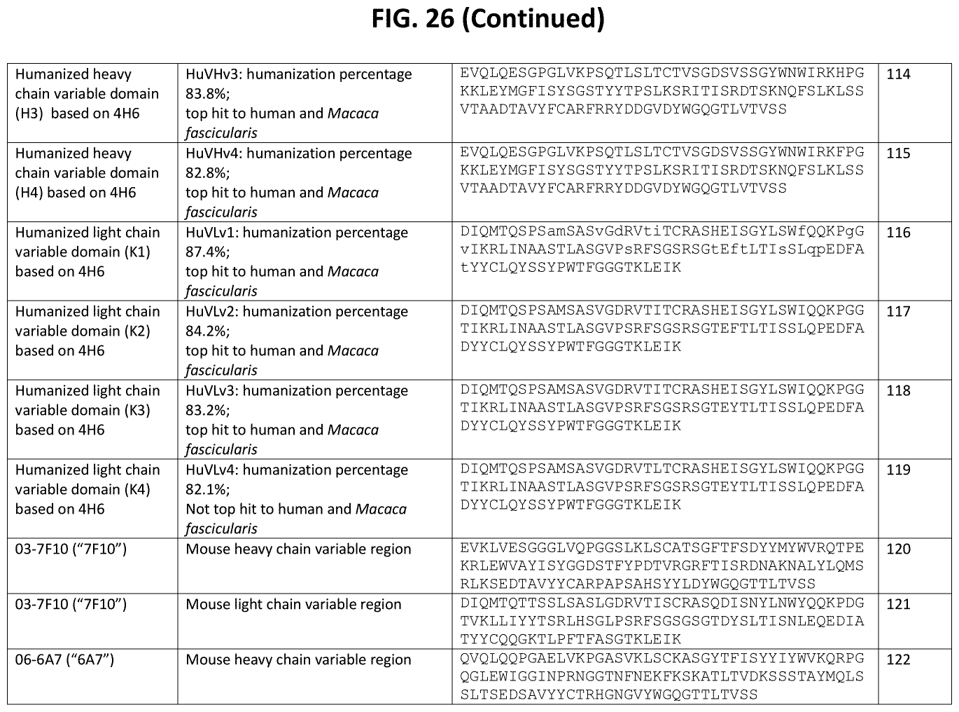

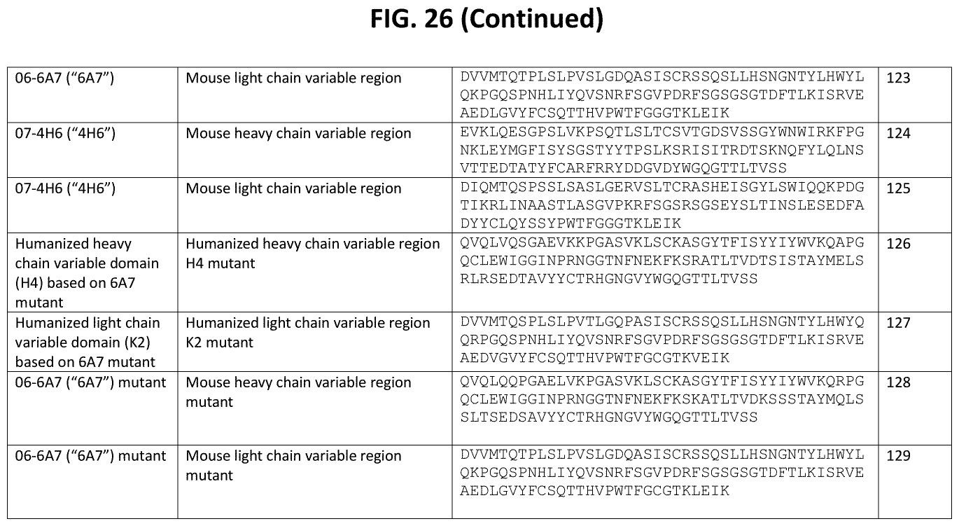

As shown in FIG. 26, humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

Furthermore, in some embodiments, the antibodies or antigen-binding fragments thereof described herein can also contain one, two, or three heavy chain variable region CDRs selected from the group of SEQ ID NOs: 59-61, SEQ ID NOs: 65-67, SEQ ID NOs: 71-73, SEQ ID NOs: 77-79, SEQ ID NOs: 83-85, and SEQ ID NOs: 89-91,; and/or one, two, or three light chain variable region CDRs selected from the group of SEQ ID NOs: 62-64, SEQ ID NOs: 68-70, SEQ ID NOs: 74-76, SEQ ID NOs: 80-82, SEQ ID NOs: 86-88, and SEQ ID NOs: 92-94.

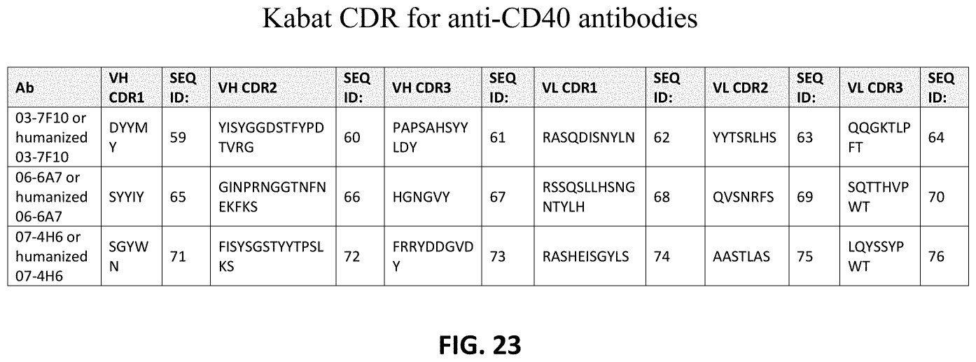

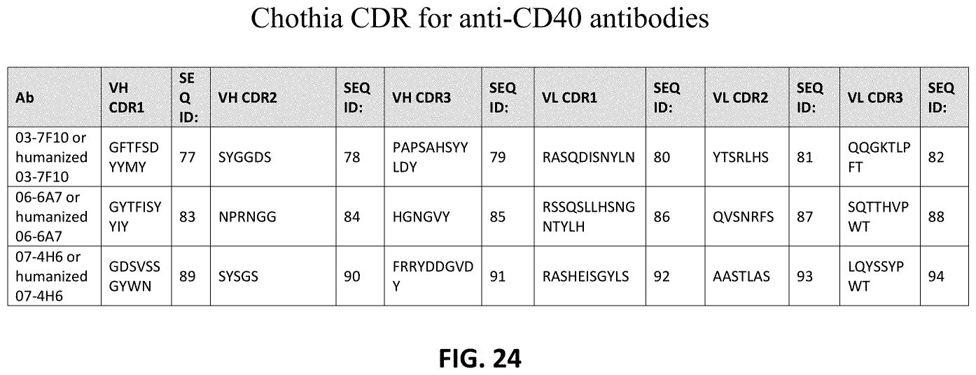

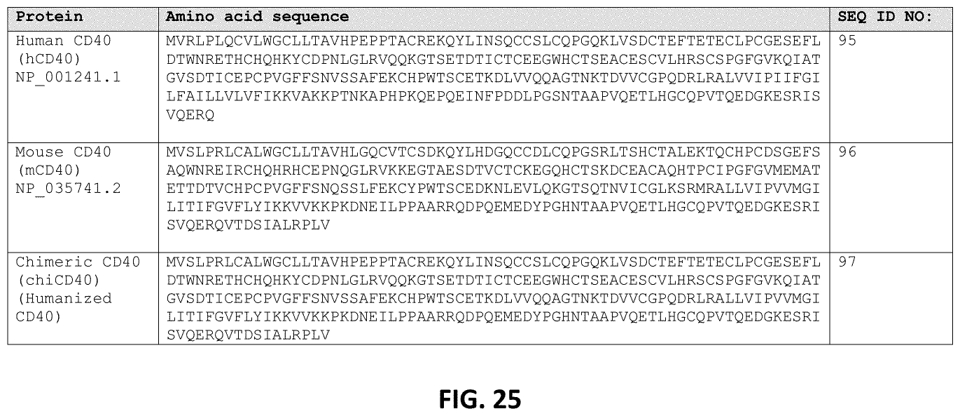

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 23 (Kabat CDR) and FIG. 24 (Chothia CDR).

In some embodiments, the antibody or an antigen-binding fragment described herein can contain a heavy chain variable domain containing one, two, or three of the CDRs of SEQ ID NO: 59 with zero, one or two amino acid insertions, deletions, or substitutions; SEQ ID NO: 60 with zero, one or two amino acid insertions, deletions, or substitutions; SEQ ID NO: 61 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 65 with zero, one or two amino acid insertions, deletions, or substitutions; SEQ ID NO: 66 with zero, one or two amino acid insertions, deletions, or substitutions; SEQ ID NO: 67 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 71 with zero, one or two amino acid insertions, deletions, or substitutions; SEQ ID NO: 72 with zero, one or two amino acid insertions, deletions, or substitutions; SEQ ID NO: 73 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 77 with zero, one or two amino acid insertions, deletions, or substitutions; SEQ ID NO: 78 with zero, one or two amino acid insertions, deletions, or substitutions; SEQ ID NO: 79 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 83 with zero, one or two amino acid insertions, deletions, or substitutions; SEQ ID NO: 84 with zero, one or two amino acid insertions, deletions, or substitutions; SEQ ID NO: 85 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 89 with zero, one or two amino acid insertions, deletions, or substitutions; SEQ ID NO: 90 with zero, one or two amino acid insertions, deletions, or substitutions; SEQ ID NO: 91 with zero, one or two amino acid insertions, deletions, or substitutions.

In some embodiments, the antibody or an antigen-binding fragment described herein can contain a light chain variable domain containing one, two, or three of the CDRs of SEQ ID NO: 62 with zero, one or two amino acid insertions, deletions, or substitutions; SEQ ID NO: 63 with zero, one or two amino acid insertions, deletions, or substitutions; SEQ ID NO: 64 with zero, one or two amino acid insertions, deletions, or substitutions.

In some embodiments, the antibody or an antigen-binding fragment described herein can contain a light chain variable domain containing one, two, or three of the CDRs of SEQ ID NO: 68 with zero, one or two amino acid insertions, deletions, or substitutions; SEQ ID NO: 69 with zero, one or two amino acid insertions, deletions, or substitutions; SEQ ID NO: 70 with zero, one or two amino acid insertions, deletions, or substitutions.

In some embodiments, the antibody or an antigen-binding fragment described herein can contain a light chain variable domain containing one, two, or three of the CDRs of SEQ ID NO: 74 with zero, one or two amino acid insertions, deletions, or substitutions; SEQ ID NO: 75 with zero, one or two amino acid insertions, deletions, or substitutions; SEQ ID NO: 76 with zero, one or two amino acid insertions, deletions, or substitutions.

In some embodiments, the antibody or an antigen-binding fragment described herein can contain a light chain variable domain containing one, two, or three of the CDRs of SEQ ID NO: 80 with zero, one or two amino acid insertions, deletions, or substitutions; SEQ ID NO: 81 with zero, one or two amino acid insertions, deletions, or substitutions; SEQ ID NO: 82 with zero, one or two amino acid insertions, deletions, or substitutions.

In some embodiments, the antibody or an antigen-binding fragment described herein can contain a light chain variable domain containing one, two, or three of the CDRs of SEQ ID NO: 86 with zero, one or two amino acid insertions, deletions, or substitutions; SEQ ID NO: 87 with zero, one or two amino acid insertions, deletions, or substitutions; SEQ ID NO: 88 with zero, one or two amino acid insertions, deletions, or substitutions.

In some embodiments, the antibody or an antigen-binding fragment described herein can contain a light chain variable domain containing one, two, or three of the CDRs of SEQ ID NO: 92 with zero, one or two amino acid insertions, deletions, or substitutions; SEQ ID NO: 93 with zero, one or two amino acid insertions, deletions, or substitutions; SEQ ID NO: 94 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that bind to CD40. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NOs: 98, 99, 100, or 120, and the selected VL sequence is SEQ ID NOs: 101, 102, 103, 104, or 121. In some embodiments, the selected VH sequence is SEQ ID NOs: 105, 106, 107, 108, 122, 126, or 128, and the selected VL sequence is SEQ ID NOs: 109, 110, 111, 123, 127, or 129. In some embodiments, the selected VH sequence is SEQ ID NOs: 112, 113, 114, 115, or 124, and the selected VL sequence is SEQ ID NOs: 116, 117, 118, 119, or 125.

In some embodiments, the antibody or antigen binding fragment thereof can have 3 VH CDRs that are identical to the CDRs of any VH sequences as described herein. In some embodiments, the antibody or antigen binding fragment thereof can have 3 VL CDRs that are identical to the CDRs of any VL sequences as described herein.

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs as shown in FIG. 23 or FIG. 24, or have sequences as shown in FIG. 26. When the polypeptides are paired with corresponding polypeptide (e.g., a corresponding heavy chain variable region or a corresponding light chain variable region), the paired polypeptides bind to CD40 (e.g., human CD40).

The anti-CD40 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CD40 will retain an ability to bind to CD40. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

In some embodiments, the antibody or antigen-binding fragment thereof is a bispecific antibody that comprises one or more (e.g., 1, 2, 3, or 4) scFv domains that binds to CD40 (e.g., human CD40).

In some embodiments, the anti-CD40 scFv is linked to the N-terminus of a heavy chain constant domain 2 (CH2) of an IgG (e.g., IgG4) Fc region. In some embodiments, the anti-CD40 scFv is linked to the C-terminus of an IgG (e.g., IgG1 or IgG4) Fc region. In some embodiments, the anti-CD40 scFv is inserted to the Fc region of an IgG (e.g., IgG1 or IgG4), e.g., at a 3A site. In some embodiments, one polypeptide chain of the antibody or antigen-binding fragment thereof is linked to the anti-CD40 scFv. In some embodiments, two polypeptide chains of the antibody or antigen-binding fragment thereof are linked to the anti-CD40 scFv.

In some embodiments, the anti-CD40 scFv comprises from N-terminus to C-terminus: heavy chain variable region (VH); linker peptide; and light chain variable region (VL). In some embodiments, the anti-CD40 scFv comprises from N-terminus to C-terminus: light chain variable region (VL); linker peptide; and heavy chain variable region (VH). In some embodiments, the VL-linker peptide-VH structure improves the expression (e.g., by at least or about 10%, 20%, 30%, 40%, or 50%) of the antibody or antigen-binding fragment thereof. The immunoglobulin heavy chain (VH) or immunoglobulin light chain (VL) comprises CDRs as shown in FIG. 23 or FIG. 24, or have sequences as shown in FIG. 26. In some embodiments, the linker peptide comprises a sequence that is at least 80%, 85%, 90%, 95%, or 100% identical to SEQ ID NO: 152, 153 or 154. In some embodiments, the anti-CD40 scFv comprises a sequence that is at least 80%, 85%, 90%, or 95% identical to SEQ ID NO: 155, 156, 157, or 158.

### Anti-PD-1 Antibodies and Antigen-Binding Fragments

The disclosure provides antibodies and antigen-binding fragments thereof that specifically bind to PD-1. The anti-PD-1/CD40 antigen-binding protein construct (e.g., bispecific antibodies) can include an antigen binding site that is derived from these antibodies.

The antibodies and antigen-binding fragments described herein are capable of binding to PD-1. The disclosure provides mouse anti-PD-1 antibodies 25-1A7 (“1A7”), 18-3F1 (“3F1”), and 3-6G1 (“6G1”), and the humanized antibodies thereof.

The CDR sequences for 1A7, and 1A7 derived antibodies (e.g., humanized antibodies) include CDRs of the heavy chain variable domain, SEQ ID NOs: 1-3, and CDRs of the light chain variable domain, SEQ ID NOs: 4-6 as defined by Kabat numbering. The CDRs can also be defined by Chothia system. Under the Chothia numbering, the CDR sequences of the heavy chain variable domain are set forth in SEQ ID NOs: 19-21, and CDR sequences of the light chain variable domain are set forth in SEQ ID NOs: 22-24.

Similarly, the CDR sequences for 3F1, and 3F1 derived antibodies include CDRs of the heavy chain variable domain, SEQ ID NOs: 7-9, and CDRs of the light chain variable domain, SEQ ID NOs: 10-12, as defined by Kabat numbering. Under Chothia numbering, the CDR sequences of the heavy chain variable domain are set forth in SEQ ID NOs: 25-27, and CDRs of the light chain variable domain are set forth in SEQ ID NOs: 28-30.

The CDR sequences for 6G1, and 6G1 derived antibodies include CDRs of the heavy chain variable domain, SEQ ID NOs: 13-15, and CDRs of the light chain variable domain, SEQ ID NOs: 16-18, as defined by Kabat numbering. Under Chothia numbering, the CDR sequences of the heavy chain variable domain are set forth in SEQ ID NOs: 31-33, and CDRs of the light chain variable domain are set forth in SEQ ID NOs: 34-36.

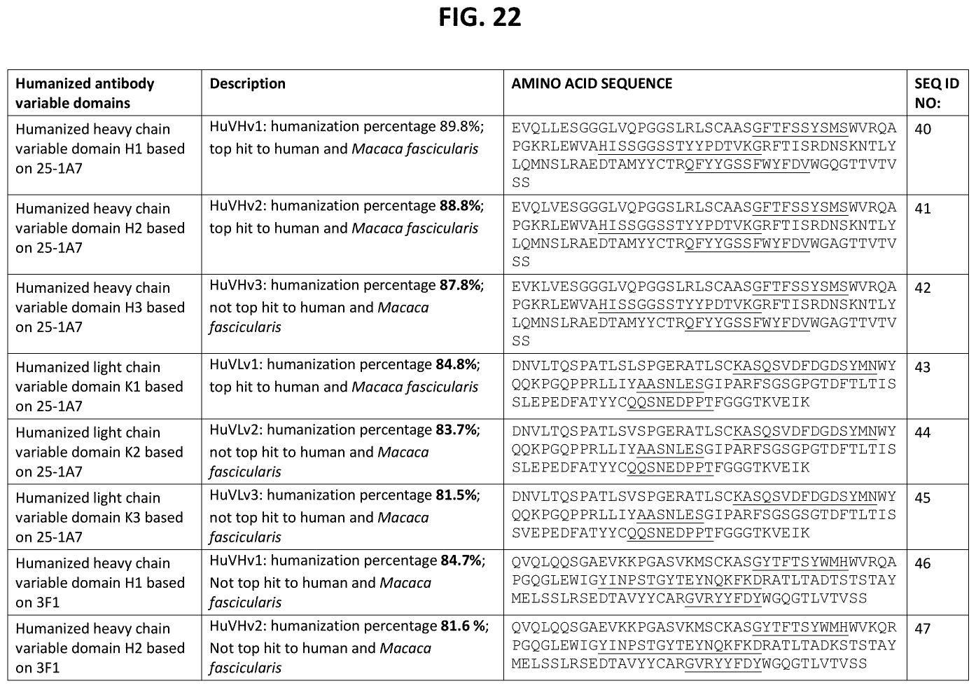

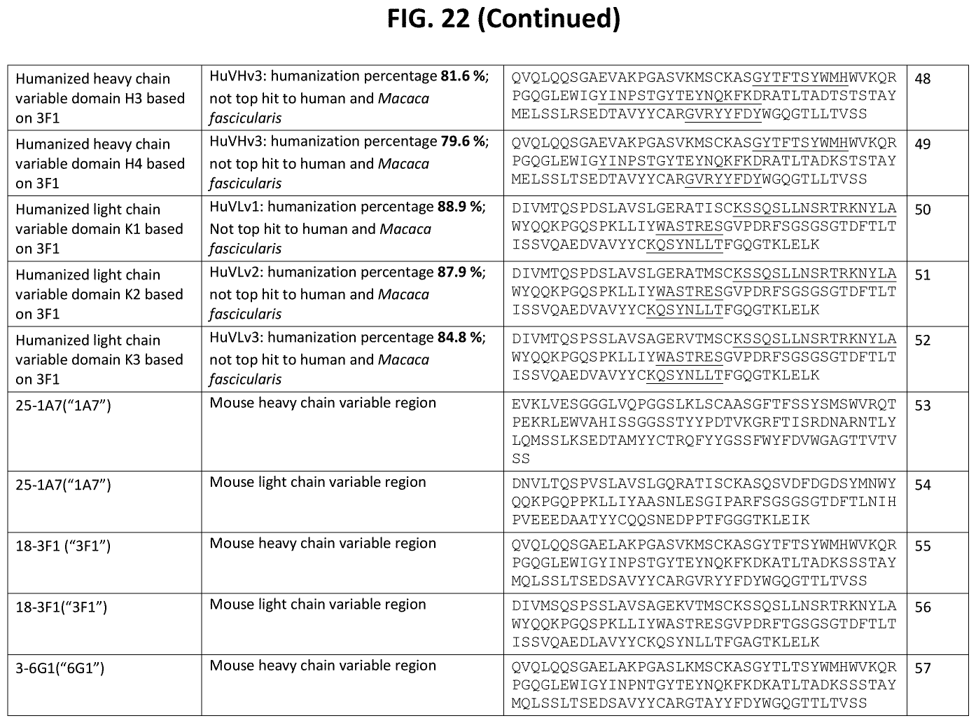

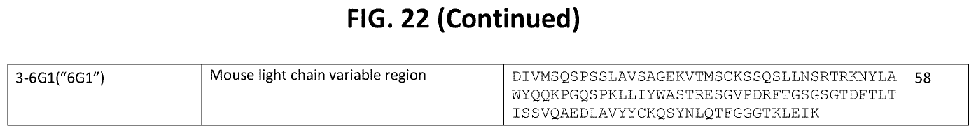

The amino acid sequence for heavy chain variable region and light variable region of humanized antibodies are also provided. As there are different ways to humanize the mouse antibody (e.g., sequence can be substituted by different amino acids), the heavy chain and the light chain of an antibody can have more than one version of humanized sequences. FIG. 22 provides humanization percentages for these humanized sequences.

The amino acid sequences for the heavy chain variable region of humanized 1A7 antibody are set forth in SEQ ID NOs: 40-42. The amino acid sequences for the light chain variable region of humanized 1A7 antibody are set forth in SEQ ID NOs: 43-45. Any of these heavy chain variable region sequences (SEQ ID NOs: 40-42) can be paired with any of these light chain variable region sequences (SEQ ID NOs: 43-45).

Similarly, the amino acid sequences for the heavy chain variable region of humanized 3F1 antibody are set forth in SEQ ID NOs: 46-49. The amino acid sequences for the light chain variable region of humanized 3F1 antibody are set forth in SEQ ID NOs: 50-52. Any of these heavy chain variable region sequences (SEQ ID NOs: 46-49) can be paired with any of these light chain variable region sequences (SEQ ID NOs: 50-52).

Furthermore, in some embodiments, the antibodies or antigen-binding fragments thereof described herein can also contain one, two, or three heavy chain variable region CDRs selected from the group of SEQ ID NOs: 1-3, SEQ ID NOs: 7-9, SEQ ID NOs: 13-15, SEQ ID NOs: 19-21, SEQ ID NOs: 25-27, and SEQ ID NOs: 31-33; and/or one, two, or three light chain variable region CDRs selected from the group of SEQ ID NOs: 4-6, SEQ ID NOs: 10-12, SEQ ID NOs: 16-18, SEQ ID NOs: 22-24, SEQ ID NOs: 28-30, and SEQ ID NOs: 34-36.

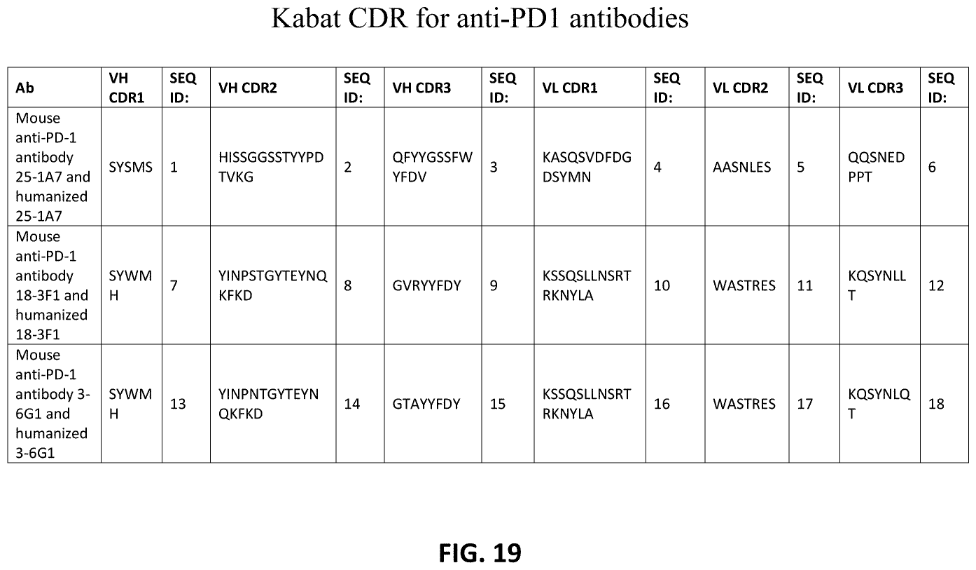

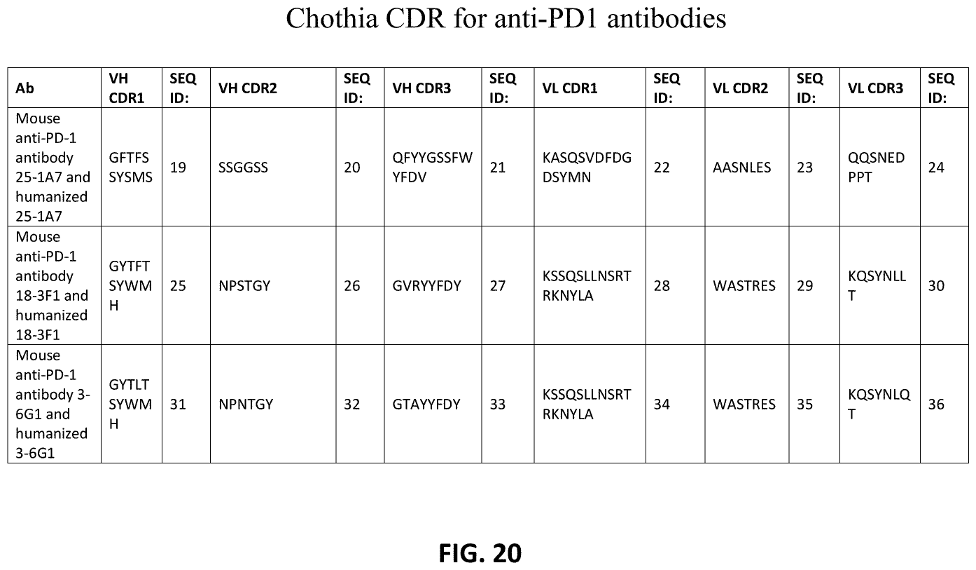

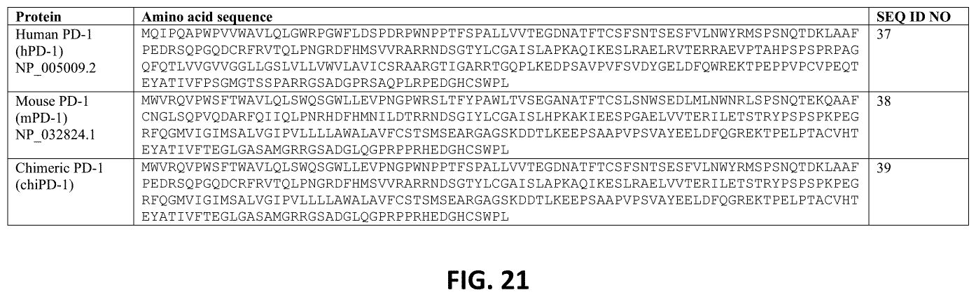

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 19 (Kabat CDR) and FIG. 20 (Chothia CDR).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a light chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a light chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a light chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a light chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a light chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that binds to PD-1. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 40, 41, 42, or 53, and the selected VL sequence is SEQ ID NO: 43, 44, 45, or 54. In some embodiments, the selected VH sequence is SEQ ID NO: 46, 47, 48, 49, or 55, and the selected VL sequence is SEQ ID NO: 50, 51, 52, or 56. In some embodiments, the selected VH sequence is SEQ ID NO: 57, and the selected VL sequence is SEQ ID NO: 58.

In some embodiments, the antibody or antigen binding fragments thereof can have 3 VH CDRs that are identical to the CDRs of any VH sequences as described herein. In some embodiments, the antibody or antigen binding fragments thereof can have 3 VL CDRs that are identical to the CDRs of any VL sequences as described herein.

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs as shown in FIG. 19 or FIG. 20, or have sequences as shown in FIG. 22. When the polypeptides are paired with corresponding polypeptide (e.g., a corresponding heavy chain variable region or a corresponding light chain variable region), the paired polypeptides bind to PD-1.

The anti-PD-1 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to PD-1 will retain an ability to bind to PD-1. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

### Antibody Characteristics

The anti-PD1, anti-CD40, or anti-PD-1/CD40 antigen-binding protein construct (e.g., antibodies, bispecific antibodies, or antibody fragments thereof) can include an antigen binding site that is derived from any anti-PD-1 antibody, anti-CD40 antibody, or any antigen-binding fragment thereof as described herein.

The antibodies, or antigen-binding fragments thereof described herein can bind to PD-1, and block the binding between PD-1 and PD-L1, and/or the binding between PD-1 and PD-L2. By blocking the binding between PD-1 and PD-L1, and/or the binding between PD-1 and PD-L2, the anti-PD-1/CD40 antibodies disrupts the PD-1 inhibitory pathway (e.g., by blocking PD-1 and PD-L1 interaction) and upregulates the immune response.

General techniques can be used to measure the affinity of an antibody for an antigen include, e.g., ELISA, RIA, and surface plasmon resonance (SPR). Affinities can be deduced from the quotient of the kinetic rate constants (KD=koff/ka). In some implementations, the antibodies, the antigen-binding fragments thereof, or the antigen-binding protein construct (e.g., bispecific antibody), can bind to PD-1 (e.g., human PD-1, mouse PD-1, and/or chimeric PD-1) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.0001 s⁻¹, or greater than 0.00001 s⁻¹. In some embodiments, the dissociation rate (koff) is less than 1.4×10⁻³ s⁻¹, 1.3×10⁻³ s⁻¹, or 1.2×10⁻³ s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (ka) is less than 1×10⁵/Ms, less than 1×10⁶/Ms, or less than 1×10⁷/Ms. In some embodiments, kinetic association rates (ka) is greater than 1.0×10⁵/Ms, 1.2×10⁵/Ms, or 1.4×10⁵/Ms.

In some embodiments, the antibodies, the antigen-binding fragments thereof, or the antigen-binding protein construct (e.g., bispecific antibody) can bind to PD-1 (e.g., human PD-1, mouse PD-1, and/or chimeric PD-1) with a KD of less than 1×10⁻⁶M, less than 1×10⁻⁷M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻¹⁰ M, greater than 1×10⁻⁸M, greater than 1×10⁻⁹ M, or greater than 1×10⁻¹⁰ M. In some embodiments, the antibody binds to human PD-1 with KD less than or equal to about 10 nM, 9.5 nM, 9 nM, 8.5 nM, or 8 nM.

The anti-PD-1/CD40 antigen-binding protein construct (e.g., bispecific antibodies) can also include an antigen binding site that is derived from any anti-CD40 antibody or antigen-binding fragment thereof as described herein. In some embodiments, the anti-PD1/CD40 antibodies or antigen-binding fragments thereof described herein can block the binding between CD40 and CD40L. In some embodiments, by binding to CD40, the antibody can also promote CD40 signaling pathway and upregulates the immune response. Thus, in some embodiments, the antibodies or antigen-binding fragments thereof as described herein are CD40 agonist. In some embodiments, the antibodies or antigen-binding fragments thereof are CD40 antagonist.

In some implementations, the antibodies, the antigen-binding fragments thereof, or the antigen-binding protein constructs (e.g., bispecific antibody) can bind to CD40 (e.g., human CD40, mouse CD40, and/or chimeric CD40) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.0001 s⁻¹, or greater than 0.00001 s⁻¹. In some embodiments, the dissociation rate (koff) is less than 5×10⁻⁴ s⁻¹, 4×10⁻⁴ s⁻¹, or 3×10⁻⁴ s⁻¹. n some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (ka) is less than 1×10⁵/Ms, less than 1×10⁶/Ms, or less than 1×10⁷/Ms. In some embodiments, kinetic association rates (ka) is greater than 1.0×10⁵/Ms, 1.5×10⁵/Ms, 2×10⁵/Ms, or 2.5×10⁵/Ms.

Affinities can be deduced from the quotient of the kinetic rate constants (KD=koff/ka). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. In some embodiments, the antibody binds to human CD40 with KD less than or equal to about 3.5 nM, 3 nM, 2.5 nM, 2 nM, or 1.5 nM.

In some embodiments, the antibodies, the antigen-binding fragments thereof, or the antigen-binding protein constructs (e.g., bispecific antibody) as described herein can block PD-1/PD-L1 pathway with a EC50 value (e.g., determined by reporter cell activation assay) that is about 50%, about 80%, about 100%, about 2 folds, about 3 folds, about 4 folds, or about 5 folds as compared to the EC50 value of an PD-1 monoclonal antibody (e.g., 1A7-H2K3-IgG4).

In some embodiments, the antibodies, the antigen-binding fragments thereof, or the antigen-binding protein constructs (e.g., bispecific antibody) as described herein can block PD-1/PD-L1 pathway with a EC50 value (e.g., determined by reporter cell activation assay) that is less than or about 100 μm/ml, 50 μm/ml, 40 μm/ml, 30 μm/ml, 20 μg/ml, 10 μm/ml, 5 μm/ml, 4 μg/ml, 3 μg/ml, 2 μm/ml, or 1 μm/ml.

In some embodiments, the antibodies, the antigen-binding fragments thereof, or the antigen-binding protein constructs (e.g., bispecific antibody) as described herein can bridge T cells (e.g., expressing PD-1) and antigen-presenting cells (e.g., expressing CD40) with a EC50 value (e.g., determined by reporter cell activation assay) that is less than or about 1 μm/ml, 0.5 μg/ml, 0.1 μm/ml, 0.05 μm/ml, 0.04 μm/ml, 0.03 μm/ml, or 0.02 μm/ml.

In some embodiments, the antibodies, the antigen-binding fragments thereof, or the antigen-binding protein constructs (e.g., bispecific antibody) as described herein can activate antigen-presenting cells (e.g., activating CD40 pathway in APC cells). In some embodiments, the presence of PD-1 expressing cells can increase APC cell activation (e.g., in trans activation) by at least or about 1 fold, 2 folds, 5 folds, 10 folds, 20 folds, 50 folds, 100 folds, 200 folds, 500 folds, or more as compared to that when the PD-1 expressing cells are absent.

In some embodiments, bridging of cells expressing PD-1 or other targets (e.g., T cells, B cells, NK cells, or myeloid cells) and APC cells by the antibodies, the antigen-binding fragments thereof, or the antigen-binding protein constructs (e.g., bispecific antibody) as described herein can stimulate CD40 clustering on APC cells by at least 10%, 20%, 30%, 40%, 50%, 60%, 70%, 80%, 90%, 100%, 2 folds, 3 folds, 5 folds, 10 folds, or 20 folds.

In some embodiments, the antibodies, the antigen-binding fragments thereof, or the antigen-binding protein constructs (e.g., bispecific antibody) as described herein can increase immune response, activity of CD40 or CD40 associated pathway, activity or number of APC cells (e.g., dendritic cells or macrophage), and/or activity or number of T cells (e.g., CD8+ and/or CD4+ cells) by at least 10%, 20%, 30%, 40%, 50%, 60%, 70%, 80%, 90%, 100%, 2 folds, 3 folds, 5 folds, 10 folds, or 20 folds.

In some embodiments, the antibodies, the antigen-binding fragments thereof, or the antigen-binding protein constructs (e.g., bispecific antibody) as described herein can activate APC cells (e.g., expressing CD40) by FCγRIIB with a EC50 value (e.g., determined by reporter cell activation assay) that is less than or about 1 μm/ml, 0.5 μm/ml, 0.1 μm/ml, 0.05 μm/ml, 0.04 μg/ml, 0.03 μm/ml, or 0.02 μm/ml.

In some embodiments, the antibodies, the antigen-binding fragments thereof, or the antigen-binding protein constructs (e.g., bispecific antibody) as described herein can decrease the FCγRIIB receptor-mediated cell (e.g., APC cell) activation by at least 10%, 20%, 30%, 40%, 50%, 60%, 70%, 80%, 90%, 100%, 2 folds, 3 folds, 5 folds, 10 folds, 20 folds, 50 folds, 100 folds, or 500 folds as compared to that of an CD40 monoclonal antibody (e.g., 6A7-H4K2-IgG2).

In some embodiments, the antibodies, the antigen-binding fragments thereof, or the antigen-binding protein constructs (e.g., bispecific antibody) as described herein are CD40 agonist. In some embodiments, the antibodies, the antigen-binding fragments thereof, or the antigen-binding protein constructs (e.g., bispecific antibody) can increase CD40 signal transduction in a target cell that expresses CD40.

The blocking effects or cell activation of the antibodies, the antigen-binding fragments thereof, or the antigen-binding protein constructs (e.g., bispecific antibody) can be measured by EC50. Half-maximal effective concentration (EC50) refers to the concentration of the agent which induces a response halfway between the baseline and maximum. In some embodiments, the EC50 is less than or about 50, 40, 30, 20, 10, 5, 1, 0.5, 0.2, 0.1, 0.05, 0.04, 0.03, 0.02, 0.01, 0.008, 0.006, 0.005, or 0.001 μg/ml.

In some embodiments, the antibodies, the antigen-binding fragments thereof, or the antigen-binding protein constructs (e.g., bispecific antibody) as described herein can amplify immune response signals in the tumor microenvironment or tumor-draining lymph node by at least 10%, 20%, 30%, 40%, 50%, 60%, 70%, 80%, 90%, 100%, 2 folds, 3 folds, 5 folds, 10 folds, or 20 folds. In some embodiments, the antibodies, the antigen-binding fragments thereof, or the antigen-binding protein constructs (e.g., bispecific antibody) as described herein can decrease overall immune activation by at least 10%, 20%, 30%, 40%, 50%, 60%, 70%, 80%, 90%, 100%, 2 folds, 3 folds, 5 folds, 10 folds, or 20 folds.

In some embodiments, the antibodies, the antigen-binding fragments thereof, or the antigen-binding protein constructs (e.g., bispecific antibody) as described herein can have similar tumor inhibition effect (e.g., represented by TGI%) with a dosage level that is less than or about 90%, 80%, 70%, 60%, 50%, or less than that of monoclonal antibodies targeting the same antigens (e.g., monoclonal antibodies comprising the same heavy chain variable region and/or light chain variable region).

In some embodiments, the antibodies, the antigen-binding fragments thereof, or the antigen-binding protein constructs (e.g., bispecific antibody) as described herein have a tumor growth inhibition effect that is at least or about 1 fold, 2 folds, 3 folds, 5 folds, 10 folds, 20 folds, or 50 folds higher than that of monoclonal antibodies targeting the same antigens when administered at a similar dosage level. In some embodiments, the presence of PD-1 expressing cells can increase the tumor growth inhibition effect by at least or about 1 fold, 2 folds, 5 folds, 10 folds, 20 folds, 50 folds, 100 folds, 200 folds, 500 folds, or more as compared to that when the PD-1 expressing cells are absent.

In some embodiments, the antibodies, the antigen-binding fragments thereof, or the antigen-binding protein constructs (e.g., bispecific antibody) as described herein have a decreased in vivo toxicity (e.g., blood AST, ALT level) by at least or about 10%, 20%, 30%, 40%, or 50% as compared to monoclonal antibodies targeting the same antigens when administered at a similar dosage level.

In some embodiments, animals (e.g., mice) administered with the antibodies, the antigen-binding fragments thereof, or the antigen-binding protein constructs (e.g., bispecific antibody) as described herein have a decreased inflammatory level (e.g., degree of lesion in liver and/or kidney) by at least 10%, 20%, 30%, 40%, or 50% as compared to animals administered with monoclonal antibodies targeting the same antigens at a similar dosage level.

In some embodiments, the antibodies, the antigen-binding fragments thereof, or the antigen-binding protein constructs (e.g., the anti-CD40 antibody, the anti-PD-1 antibody, or the bispecific antibody) has a tumor growth inhibition percentage (TGI%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI% can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI% or TGI_{TV}%) is calculated using the following formula:

TGI(%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibody, the antigen-binding fragment thereof, or the antigen-binding protein construct (e.g., bispecific antibody) has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4. In some embodiments, the antibody, the antigen-binding fragment thereof, or the antigen-binding protein construct (e.g., bispecific antibody) does not have a functional Fc region. For example, the antibodies or antigen binding fragments are Fab, Fab′, F(ab′)2, and Fv fragments.

### Antibodies and Antigen Binding Fragments

The present disclosure provides antibodies, the antigen-binding fragments thereof, or the antigen-binding protein constructs (e.g., bispecific antibodies). The antigen-binding protein construct (e.g., bispecific antibody) can comprise an anti-CD40 antibody or antigen-binding fragment thereof, and anti-PD-1 antibody or antigen-binding fragment thereof. These antigen-binding protein constructs (e.g., bispecific antibody), anti-CD40 antibodies, anti-PD-1 antibodies, and antigen-binding fragments thereof can have various forms.

In general, antibodies (also called immunoglobulins) are made up of two classes of polypeptide chains, light chains and heavy chains. A non-limiting antibody of the present disclosure can be an intact, four immunoglobulin chain antibodies comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgEl, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen binding site.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety. Unless specifically indicated in the present disclosure, Kabat numbering is used in the present disclosure as a default.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding sites (Antigen binding sites: A and B), and the two antigen binding sites can bind to the respective target antigens with different affinities.

In some embodiments, the antibodies, the antigen-binding fragments thereof, or the antigen-binding protein constructs (e.g., bispecific antibodies) can bind to two different antigens or two different epitopes.

In some embodiments, the antibodies, the antigen-binding fragments thereof, or the antigen-binding protein constructs (e.g., bispecific antibodies) can comprises one, two, or three heavy chain variable region CDRs selected from FIGS. 19, 20, 23, and 24. In some embodiments, the antibodies, the antigen-binding fragments thereof, or the antigen-binding protein constructs (e.g., bispecific antibodies) can comprises one, two, or three light chain variable region CDRs selected from FIGS. 19, 20, 23, and 24.

In some embodiments, the antibodies, the antigen-binding fragments thereof, or the antigen-binding protein constructs (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

Single-chain Fv or (scFv) antibody fragments comprise the VH and VL domains (or regions) of antibody, wherein these domains are present in a single polypeptide chain. Generally, the Fv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding sites. Linear antibodies can be bispecific or monospecific.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies, the antigen-binding fragments thereof, or the antigen-binding protein constructs (e.g., bispecific antibodies) described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

The antibodies, the antigen-binding fragments thereof, or the antigen-binding protein constructs (e.g., bispecific antibodies) can also have various forms. Many different formats of antigen binding constructs are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the antigen-binding protein construct is a BiTe, a (scFv)2, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the antigen-binding protein construct is a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv2.

In some embodiments, the antigen-binding protein construct can be a TrioMab. In a TrioMab, the two heavy chains are from different species, wherein different sequences restrict the heavy-light chain pairing.

In some embodiments, the antigen-binding protein construct has two different heavy chains and one common light chain. Heterodimerization of heavy chains can be based on the knob-in-holes or some other heavy chain pairing technique.

In some embodiments, CrossMAb technique can be used produce bispecific antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi-and multispecific antibodies.” MAbs. Vol. 8. No. 6. Taylor & Francis, 2016, which is incorporated by reference in its entirety. In some embodiments, the CH1 in the heavy chain and the CL domain in the light chain are swapped.

In some embodiments, the antigen-binding protein construct can be a 2:1 CrossMab. An additional Fab-fragment is added to the N-terminus of its VH domain of the CrossMab. The added Fab-fragment to the CrossMab increases the avidity by enabling bivalent binding.

In some embodiments, the antigen-binding protein construct can be a 2:2 CrossMab. This tetravalent bispecific antibody generated by fusing a Fab-fragment to each C-terminus of a CrossMab. These Fab-fragments can be crossed: their CH1 is switched with their CL. VH is fused to their CL and the VL to the CH1. CrossMab technique in Fab-fragments ensure specific pairing. Avidity can be enhanced by double bivalent binding.

The antigen-binding protein construct can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the antigen-binding protein construct can be have a IgG-IgG format. Two intact IgG antibodies are conjugated by chemically linking the C-terminals of the heavy chains.

The antigen-binding protein construct can also have a Fab-scFv-Fc format. In Fab-scFv-Fc format, a light chain, heavy chain and a third chain containing the Fc region and the scFv are assembled. It can ensure efficient manufacturing and purification.

In some embodiments, antigen-binding protein construct can be a TF. Three Fab fragments are linked by disulfide bridges. The TF format does not have an Fc region.

ADAPTIR has two scFvs bound to each sides of an Fc region. It abandons the intact IgG as a basis for its construct, but conserves the Fc region to extend the half-life and facilitate purification.

Bispecific T cell Engager (“BiTE”) consists of two scFvs, VLA VHA and VHB VLB on one peptide chain. It has only binding domains, no Fc region.

In BiTE-Fc, an Fc region is fused to the BiTE construct. The addition of Fc region enhances half-life leading to longer effective concentrations, avoiding continuous IV.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fc, an Fc region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fc region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In modular scFv-scFv-scFv, one scFv directed against the TAA is tagged with a short recognizable peptide is assembled to a bsAb consisting of two scFvs, one directed against CD3 and one against the recognizable peptide.

In ImmTAC, a stabilized and soluble T cell receptor is fused to a scFv recognizing CD3. By using a TCR, the ImmTAC is suitable to target processed, e.g. intracellular, proteins.

Tri-specific nanobody has two single variable domains (nanobodies) with an additional module for half-life extension. The extra module is added to enhance half-life.

In Trispecific Killer Engager (TriKE), two scFvs are connected via polypeptide linkers incorporating human IL-15. The linker to IL-15 is added to increase survival and proliferation of NKs.

In one aspect, the disclosure provides a bispecific antibody or antigen-binding fragment thereof, comprising:

(a) a heavy chain polypeptide comprising, preferably from N-terminus to C-terminus, a first light chain variable region (VL1), a first linker peptide sequence, a second heavy chain variable region (VH2), an optional heavy chain constant region 1 (CH1), a heavy chain constant region 2 (CH2), and a heavy chain constant region 3 (CH3); and

(b) a light chain polypeptide comprising, preferably from N-terminus to C-terminus, a second light chain variable region (VL2), a second linker peptide sequence, a first heavy chain variable region (VH1), a first light chain variable region (VL1), and an optional light chain constant region (CL).

In some embodiments, the VH1 and VL1 interact with each other, forming a first antigen-binding site that specifically binds to PD-1. In some embodiments, the VH2 and VL2 interact with each other, forming a first antigen-binding site that specifically binds to CD40.

In some embodiments, the VH1 and VL1 interact with each other, forming a first antigen-binding site that specifically binds to CD40. In some embodiments, the VH2 and VL2 interact with each other, forming a first antigen-binding site that specifically binds to PD-1.

In some embodiments, the first and/or second linker peptide comprises a sequence that is at least 80%, 85%, 90%, 95%, or 100% identical to SEQ ID NO: 205. In some embodiments, the CH1, CH2, CH3, and/or CL domains are from human IgG (e.g., IgG1 or IgG4). In some embodiments, the human IgG (e.g., IgG1 or IgG4) comprises KIH and/or LALA mutations.

In some embodiments, the bispecific antibody or antigen-binding fragment thereof further comprises: a second heavy chain polypeptide that is at least 90%, 95%, or 100% identical to the first heavy chain polypeptide; and a second light chain polypeptide that is at least 90%, 95%, or 100% identical to the first light chain polypeptide.

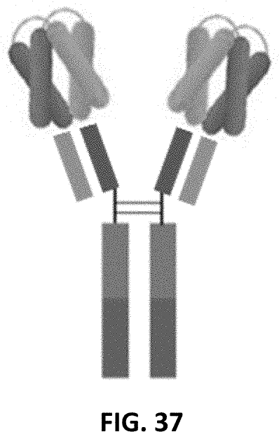

In some embodiments, the bispecific antibody or antigen-binding fragment thereof described herein has a schematic structure shown in FIG. 37. In some embodiments, the heavy chain polypeptide is set forth in SEQ ID NO: 170, and the light chain polypeptide is set forth in SEQ ID NO: 171.

### Methods of Making Antigen-Binding Protein Constructs

An isolated fragment of human protein can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, N.Y.). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibodies, the antigen-binding fragments thereof, or the antigen-binding protein constructs (e.g., bispecific antibodies). These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the Leu234Ala/Leu235Ala (EU numbering) (LALA) mutations are introduced to disrupt antibody effector functions.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, knob-into-hole (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7. No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (EU numbering). In some embodiments, one heavy chain has one or more of the following substitutions Y349C and T366W (EU numbering). The other heavy chain can have one or more the following substitutions E356C, T366S, L368A, and Y407V (EU numbering). Furthermore, a substitution (-ppcpScp-->-ppcpPcp-) can also be introduced at the hinge regions of both substituted IgG.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

Thus, in some embodiments, the amino acid residue at Kabat numbering position 83 is lysine, arginine, or histidine. In some embodiments, the amino acid residues at one or more of the positions 1, 6, 43, 81, and 105 (Kabat numbering) is aspartic acid or glutamic acid. In some embodiments, the amino acid residues at one or more of the positions 13 and 105 (Kabat numbering) is aspartic acid or glutamic acid. In some embodiments, the amino acid residues at one or more of the positions 13 and 42 (Kabat numbering) is lysine, arginine, histidine, or glycine.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bispecific antibodies from antibody fragments are also known in the art. For example, bispecific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bispecific antibody.

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HEK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lad and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished using a Blossum 62 scoring matrix with a gap penalty of 12, a gap extend penalty of 4, and a frameshift gap penalty of 5.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to PD-1. In some embodiments, a pair of vectors is provided, wherein each vector comprises one of the nucleic acids as described herein, wherein together the pair of vectors encodes the VL region and the VH region that together bind to PD-1. In some embodiments, the vector includes two of the nucleic acids as described herein, wherein the vector encodes the VL region and the VH region that together bind to CD40. In some embodiments, a pair of vectors is provided, wherein each vector comprises one of the nucleic acids as described herein, wherein together the pair of vectors encodes the VL region and the VH region that together bind to CD40.

Vectors can also be constructed to express specific antibodies or polypeptides. In some embodiments, a vector can be constructed to co-express one or more antibody polypeptide chains. In some embodiments, a vector can contain sequences of, from 5′ end to 3′ end, cytomegalovirus promotor (CMV), a sequence encoding the first polypeptide chain, polyadenylation (PolyA), CMV, a sequence encoding the second polypeptide chain, PolyA, simian vacuolating virus 40 terminator (SV40) and glutamine synthetase marker (GS). In some embodiments, a vector can be constructed to express anti-CD40 antibody scFv polypeptide chain.

### Methods of Treatment

The methods described herein include methods for the treatment of disorders associated with cancer. Generally, the methods include administering a therapeutically effective amount of engineered antibodies, the antigen-binding fragments thereof, or the antigen-binding protein constructs (e.g., bispecific antibodies) as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies, the antigen-binding fragments thereof, or the antigen-binding protein constructs (e.g., bispecific antibodies), or an antibody drug conjugates disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer.

In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the methods described herein can be used to treat a hot tumor. A hot tumor is a tumor that is likely to trigger a strong immune response. Hot tumors often have many molecules on their surface that allow T cells to attack and kill the tumor cells. The method described herein can further increase the immune response, promote the proliferation and infiltration of CD8+ T cells, and/or and reduce the number of myeloid-derived suppressor cells in the tumor.

In some embodiments, the methods described herein can be used to treat a cold tumor. A cold tumor is a tumor that is not likely to trigger a strong immune response. Cold tumors tend to be surrounded by cells that are able to suppress the immune response and keep T cells from attacking the tumor cells and killing them. The method described herein can effectively promote the proliferation, infiltration, and/or activation of DC cells, thereby increasing the activities of antigen presenting cells. In some embodiments, the methods as described herein can downregulate the expression of PD-1 in T cells, thereby further reducing the interaction between PD-1 and PD-L1 and increasing immune response. As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody, the antigen-binding fragment thereof, or the antigen-binding protein construct (e.g., a bispecific antibody) is 0.01 mg/kg to 100 mg/kg. In some embodiments, the dosage can be less than 100 mg/kg, 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, the antigen-binding fragment thereof, or the antigen-binding protein construct (e.g., a bispecific antibody), antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, the antigen-binding fragment thereof, the antigen-binding protein construct (e.g., a bispecific antibody), or antibody-drug conjugate, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, an anti-LAG-3 antibody, an anti-TIGIT antibody, an anti-BTLA antibody, or an anti-GITR antibody.

In some embodiments, in the absence of PD-1 expressing cells (e.g., T cells), a PD1/CD40 bispecific antibody cannot effectively activate CD40 signaling pathway in antigen-presenting cells (APCs).

In some embodiments, in the presence of PD-1 expressing cells, a PD1/CD40 bispecific antibody can effectively activate CD40 signaling pathway.

### Pharmaceutical Compositions and Routes of Administration

Also provided herein are pharmaceutical compositions that contain at least one (e.g., one, two, three, or four) of the antigen-binding protein constructs, antibodies (e.g., bispecific antibodies), antigen-binding fragments, or antibody-drug conjugates described herein. Two or more (e.g., two, three, or four) of any of the antigen-binding protein constructs, antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antigen-binding protein constructs, antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antigen-binding protein constructs, antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antigen-binding protein constructs, antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antigen-binding protein constructs, antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg). While these doses cover a broad range, one of ordinary skill in the art will understand that therapeutic agents, including antigen-binding protein constructs, antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1. Preparation of Anti-PD-1/CD40 Bispecific Antibody

Two types of anti-PD-1/CD40 bispecific antibodies (BsAbs) were designed. As shown in FIG. 1A, the Fab-ScFV-IgG BsAb has an anti-PD-1 arm comprising a heavy chain and a light chain, and an anti-CD40 arm comprising a single-chain variable fragment (scFv) connected to CH2 and CH3 domains of IgG. As shown in FIG. 1B, the ScFV-HC-IgG BsAb has an anti-CD40 scFv connected to each of the heavy chain C-terminus of an anti-PD-1 monoclonal antibody. Sequences related to the PD-1 antibody 1A7 are described in PCT/CN2018/077016, which is incorporated herein by reference in its entirety. Sequences related to the CD40 antibody 6A7 are described in PCT/CN2018/096494, which is incorporated herein by reference in its entirety.

Vectors expressing respective polypeptide chains of the BsAbs were co-transfected into CHO cells. After 14 days of culture, the cell supernatant was collected and purified by Protein A affinity chromatography, followed by size-exclusion chromatography (SEC) to obtain the bispecific antibodies. Constant domains of the BsAbs were selected from either human IgG1 or IgG4. In particular, mutations within IgG1, e.g., LALA (L234A/LS235A) mutations, were also introduced to reduce Fc receptor binding affinities. These Fc mutations can improve antibody safety by minimizing antibody effector functions.

Various vectors for expressing these antibodies were prepared for the following experiments. In the following experiments, the anti-PD-1 VH and VL in Fab-ScFV-IgG and ScFV-HC-IgG were derived from the 1A7-H2K3 anti-PD1 antibody. The anti-CD40 ScFV VH and VL were derived from the 6A7-H4K2 anti-CD40 antibody.

In the Fab-ScFV-IgG antibodies, knobs-into-holes (KIH) mutations were also introduced to the constant regions. The PD-1 arm of Fab-ScFV-IgG4 included the VH of 1A7-H2K3 and the IgG4 constant region with KIH mutation (SEQ ID NO: 41 and 150). The ScFV arm included the VH and VL of 6A7-H4K2 (SEQ ID NO: 108 and 110; or SEQ ID NO: 126 and 127) and the IgG4 constant region with the corresponding KIH mutation (SEQ ID NO: 151). In ScFV-HC-IgG, the anti-CD40 ScFV was added to the C terminal of the anti-PD-1 antibody with a linker sequence.

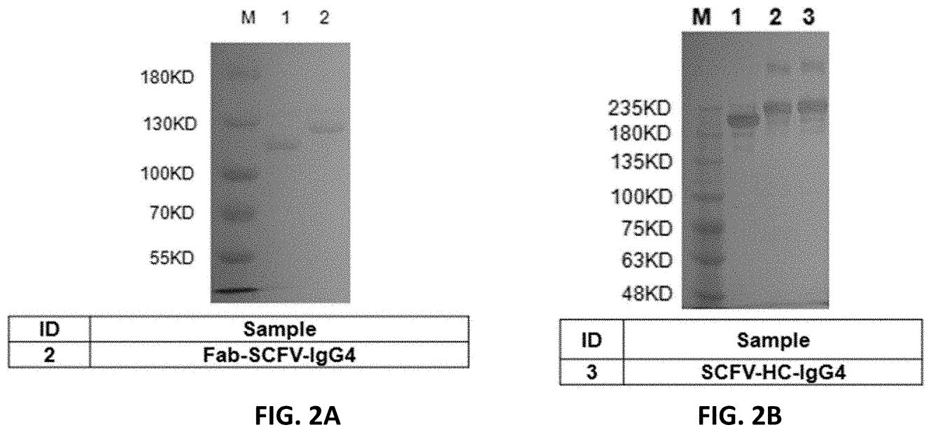

Purified BsAbs were detected by non-reducing gel electrophoresis (6% separation gel) as shown in FIGS. 2A-2B. Each lane was loaded with 2 μg protein. The results showed that all the BsAbs were expressed and correctly assembled.

### Example 2. Antibody Binding Affinity

The binding affinity of the BsAbs against human PD-1 (hPD-1) or human CD40 (hCD40) were determined by Biacore systems. Both the human PD-1 protein (human PD-1/PDCD1 protein, His Tag) and the human CD40 protein (human CD40/TNFRSFS Protein, His Tag) were purchased from ACRO Biosystems with Cat #PD1-H5221 and Cat#CDO-H5228, respectively. Results are summarized in the tables below.

TABLE 1

| | hPD-1 | | |
|---|---|---|---|
| | Kon | Koff | |
| Capture 1 Solution | (1/Ms) | (1/s) | KD (M) |
| Fab-ScFV-IgG4 | 1.20E+05 | 1.17E−03 | 9.78E−09 |
| ScFV-HC-IgG4 | 1.48E+05 | 1.22E−03 | 8.28E−09 |
| PD-1 (1A7-H2K3- | 1.46E+05 | 1.25E−03 | 8.56E−09 |
| IgG4) | | | |

TABLE 2

| | hCD40 | | |
|---|---|---|---|
| | Kon | Koff | |
| Capture 1 Solution | (1/Ms) | (1/s) | KD (M) |
| Fab-ScFV IgG4 | 2.85E+05 | 5.00E−04 | 1.75E−09 |
| ScFV-HC IgG4 | 2.61E+05 | 3.40E−04 | 1.30E−09 |
| CD40 (6A7-H4K2- | 1.05E+05 | 3.22E−04 | 3.06E−09 |
| IgG2) | | | |

The result showed that both types of bispecific antibodies had a binding affinity that was comparable to the parent monoclonal antibodies. The mutations (e.g., LALA mutations) are within the FC region therefore do not affect binding affinity.

### Example 3. Jurkat-luc-hPD1 Reporter Cell Activation Assay

The experiment was performed to test whether the BsAbs Fab-ScFV-IgG4 and ScFV-HC-IgG4 can block the PD-1/PD-L1 pathway.

Basal cells CHO-aAPC-hPD-L1 (Promega, CattJ1255) were seeded in a 96-well plate (cell density 4×10⁴ cells/well) and incubated at 37° C. overnight. The bispecific antibodies Fab-ScFV-IgG4, ScFV-HC-IgG4, and anti-PD-1 monoclonal antibody 1A7-H2K3-IgG4 were serially diluted (3-fold) with the highest concentration at 100 μm/ml. The assay buffer was RPMI 1640 medium supplemented with 10% fetal bovine serum (FBS). On the next day, effector cells Jurkat-Luc-hPD-1 (Promega, Cat#: J1255) were centrifuged at 120 g for 10 minutes, and then seeded in a 96-well plate (cell density 5×10⁴ cells/well). Next, supernatant from the plate seeded with CHO-aAPC-hPD-L1 was discarded, and then 50 μl Jurkat-Luc-hPD-1 cells together with 25 μl antibody were added to each well. The above 96-well plate was incubated in a 37° C. incubator for 6 hours. After the incubation, the plate was taken out, and 75 μl of Bio-lite Luciferase Assay Reagent (Vazyme Biotech Co., Ltd., Cat#: DD1201-02-AB) was added to incubate at room temperature for 5-10 minutes. The plate was then placed in a luminescence detector to detect the fluorescence signal. If the antibody can block the interaction between PD-1 and PD-L1, the effector cells will report a signal.

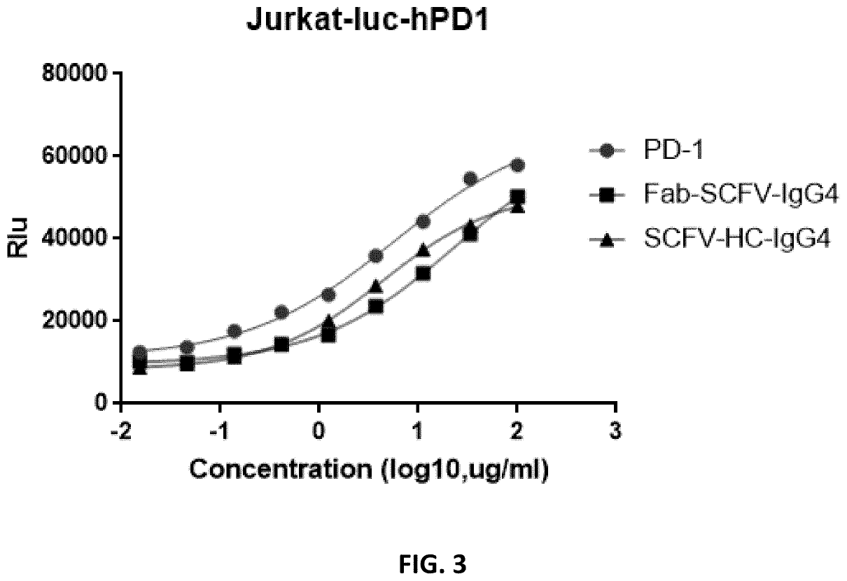

As shown in FIG. 3 and Table 3, the BsAbs exhibited blocking effect to the PD-1/PD-L1 pathway. Because Fab-ScFV-IgG4 binds to PD-1 with a single anti-PD-1 arm, as compared to the two anti-PD-1 arms of 1A7-H2K3-IgG4 and ScFV-HC-IgG4, the EC50 value of Fab-ScFV-IgG4 was relatively higher.

TABLE 3

| Antibody Name | EC50 Value (ug/ml) | R² value |
|---|---|---|
| 1A7-H2K3-IgG4 | 5.605 | 0.9968 |
| Fab-ScFV-IgG4 | 23.37 | 0.999 |
| ScFV-HC-IgG4 | 4.356 | 0.9997 |

### Example 4

### T Cell/APC Cell Bridging Effect of Bispecific Antibodies

The experiment was performed to test whether the BsAbs Fab-ScFV-IgG4 and ScFV-HC-IgG4 can bridge the T cells and APC cells using reporter cells.

Basal cells CHO-K1-hPD1 were seeded in a 96-well plate (cell density 5×10⁴ cells/well) and incubated at 37° C. overnight. The BsAbs Fab-ScFV-IgG4 and ScFV-HC-IgG4 were serially diluted (3-fold) with the highest concentration of 5 μg/ml. Meanwhile, the anti-PD-1 antibody 1A7-H2K3-IgG4 and anti-CD40 antibody 6A7-H4K2-IgG2 were combined at 1:1 ratio and then serially diluted (3-fold) with the highest concentration of 10 μm/ml (5 μm/ml for each monoclonal antibody). The assay buffer was RPMI 1640 medium supplemented with 10% fetal bovine serum (FBS). On the next day, effector cells Jurkat-Luc-hCD40 (Promega, Cat#: JA2125) were centrifuged at 120 g for 10 minutes, and then seeded in a 96-well plate (cell density 5×10⁴ cells/well). Next, supernatant from the plate seeded with CHO-K1-hPD1 was discarded, and then 50 μl Jurkat-Luc-hCD40 cells together with 25 μl antibody were added to each well. The above 96-well plate was incubated in a 37° C. incubator for 6 hours. After the incubation, the plate was taken out, and 75 μl of Bio-lite Luciferase Assay Reagent (Vazyme Biotech Co., Ltd., Cat#: DD1201-02-AB) was added to incubate at room temperature for 5-10 minutes. The plate was then placed in a luminescence detector to detect the fluorescence signal.

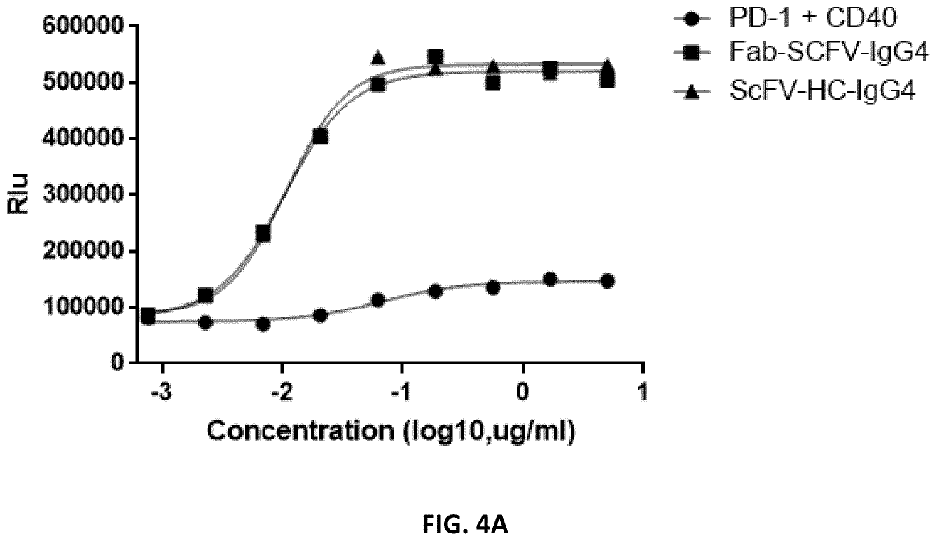

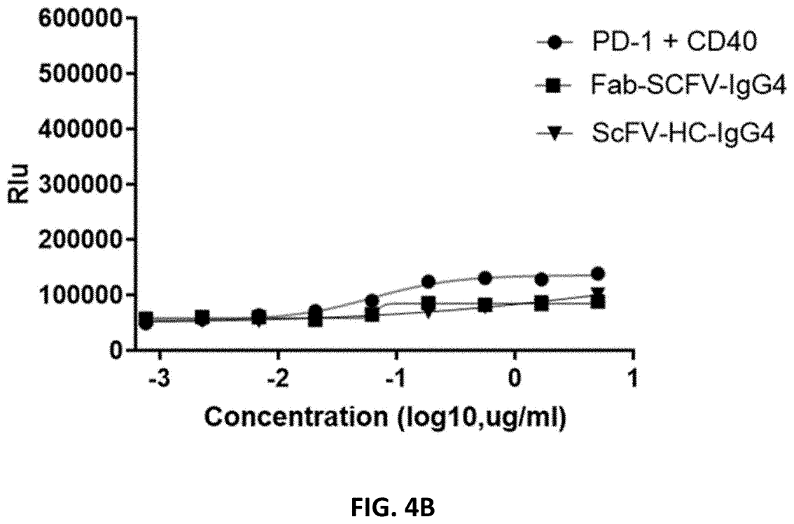

As shown in FIG. 4A, when CHO-K1-hPD1 cells were present, both Fab-ScFV-IgG4 and ScFv-HC-IgG4 activated the reporter cells in trans. By contrast, combination of the monoclonal antibodies did not activate the reporter cells. This is probably because the activation of CD40 requires the clustering of CD40, which was facilitated by the basal cells. As shown in FIG. 4B, in the absence of CHO-K1-hPD1, neither the BsAbs nor the monoclonal antibody combination activated the reporter cells. The EC50 values are shown in the table below. Thus, the tested bispecific antibodies are capable of bridging T cells and APC cells.

TABLE 4

| Antibody Name | EC50 Value (ug/ml) | R² value |
|---|---|---|
| Fab-ScFV-IgG4 | 0.0103 | 0.9938 |
| ScFV-HC-IgG4 | 0.0108 | 0.9943 |

Here, the Jurkat-Luc-hCD40 cells did not express PD-1 and was used to verify CD40 pathway activation in APC cell (e.g., dendritic cells, or macrophage). Bridging of T cells (e.g., expressing PD-1 or other targets) and APC cells by the BsAbs can stimulate CD40 clustering on APC cells, thereby amplifying immune response signals in tumor microenvironment. The results also indicates that activation of the CD40 pathway by the bispecific antibodies depends on their bridging effect with PD-1 expressing cells. As immune cells expressing PD-1 will be recruited to the tumor microenvironment and PD-1 expression is usually up-regulated in the tumor microenvironment, this mechanism can also limit immune activation largely to tumor microenvironment and reduce side effects, e.g., toxicity in liver. In addition, the tumor draining lymph nodes has both antigen presenting cells and T cells expressing PD-1. The bispecific antibodies can effectively increase immune response in the tumor draining lymph nodes. In the meantime, it can suppress suppressor activity of regulatory T cells (Tregs), thereby inhibiting systematic tolerance.

### Example 5. Fc Receptor-Mediated Reporter Cell Activation Assay

The experiment was performed to test whether the BsAbs Fab-ScFV-IgG4 and ScFV-HC-IgG4 can activate reporter cells via FcR crosslinking (e.g. via the FCγRIIB receptor).

Basal cells CHO-K1-hFcγRIM (Promega, Cat#: JA2251) were seeded in a 96-well plate (cell density 5×10⁴ cells/well) and incubated at 37° C. overnight. The anti-CD40 monoclonal antibody 6A7-H4K2-IgG2, and bispecific antibodies Fab-ScFV-IgG4, ScFV-HC-IgG4 were serially diluted (3-fold) with the highest concentration of 10 μg/ml. The assay buffer was RPMI 1640 medium supplemented with 10% fetal bovine serum (FBS). On the next day, effector cells Jurkat-Luc-hCD40 (Promega, Cat#: JA2251) were centrifuged at 120 g for 10 minutes, and then seeded in a 96-well plate (cell density 5×10⁴ cells/well). Next, supernatant from the plate seeded with CHO-K1-hFcγRIM was discarded, and then 50 μl Jurkat-Luc-hCD40 cells together with 25 μl antibody were added to each well. The above 96-well plate was incubated in a 37° C. incubator for 6 hours. After the incubation, the plate was taken out, and 75 μl of Bio-lite Luciferase Assay Reagent (Vazyme Biotech Co., Ltd., Cat#: DD1201-02-AB) was added to incubate at room temperature for 5-10 minutes. The plate was then placed in a luminescence detector to detect the fluorescence signal.

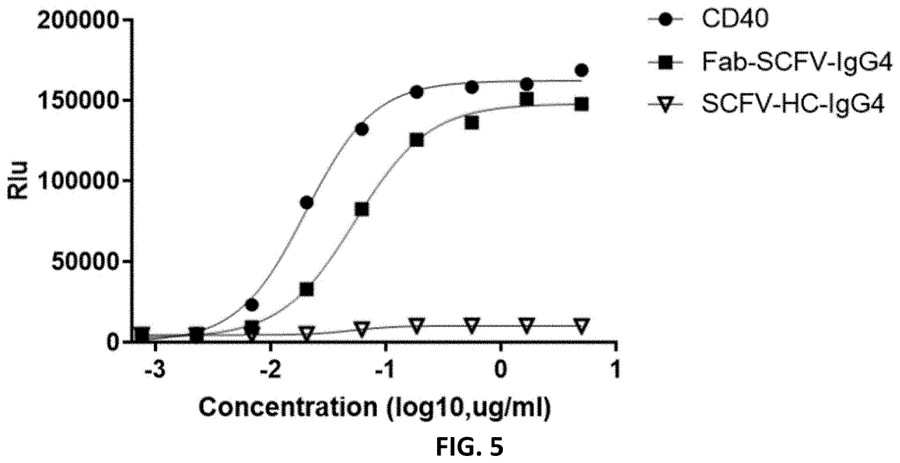

As shown in FIG. 5 and Table 5, when CHO-K1-hFcγRIIB cells were present, Fab-ScFV-IgG4 activated the reporter cells with a comparable EC50 as compared to the anti-CD40 monoclonal antibody 6A7-H4K2-IgG2. However, ScFV-HC-IgG4 did not exhibit FCγRIIB receptor-mediated reporter cell activation.

TABLE 5

| Antibody Name | EC50 Value (ug/ml) | R² value |
|---|---|---|
| 6A7-H4K2-IgG2 | 0.0203 | 0.997 |
| Fab-ScFV-IgG4 | 0.0543 | 0.998 |

CD40 antibody 6A7-H4K2-IgG2 can active the reporter cells via FCγRIIB receptor crosslinking. However, when the anti-CD40 scFv is linked to the Fc region, FCγRIIB cannot activate CD40 pathway through FCγRIIB mediated clustering. Because ScFV-HC-IgG4 did not exhibit FCγRIIB receptor-mediated reporter cell activation, ScFV-HC-IgG4 can effectively reduce toxicity in tissues expressing high level of FCγRIIB, e.g., toxicity in liver. Table 6 summarizes the in vitro activities of the two types of bispecific antibodies Fab-ScFV-IgG and ScFV-HC-IgG. Because ScFV-HC-IgG4 exhibited CD40 pathway activation in APC cell when CHO-K1-hPD1 cells were present, but did not exhibit FCγRIIB receptor-mediated T cell activation, both ScFV-HC-IgG4 and Fab-ScFV-IgG4 can be effective for treating cancer in an FcR-independent manner.

TABLE 6

| | | | mAb or |
|---|---|---|---|
| CD40 activity | Fab-ScFV-IgG4 | ScFV-HC-IgG4 | combination |
| No basal cells | No | No | No |
| CHO-K1-hPD1 | Yes | Yes | No |
| CHO-K1-hFCγRIIB | Yes | No | Yes |

### Example 6. Reporter Cell Activation by Subtypes of ScFV-HC-IgG

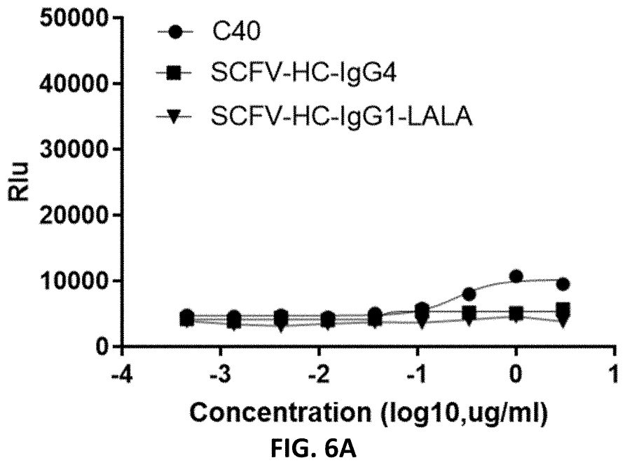

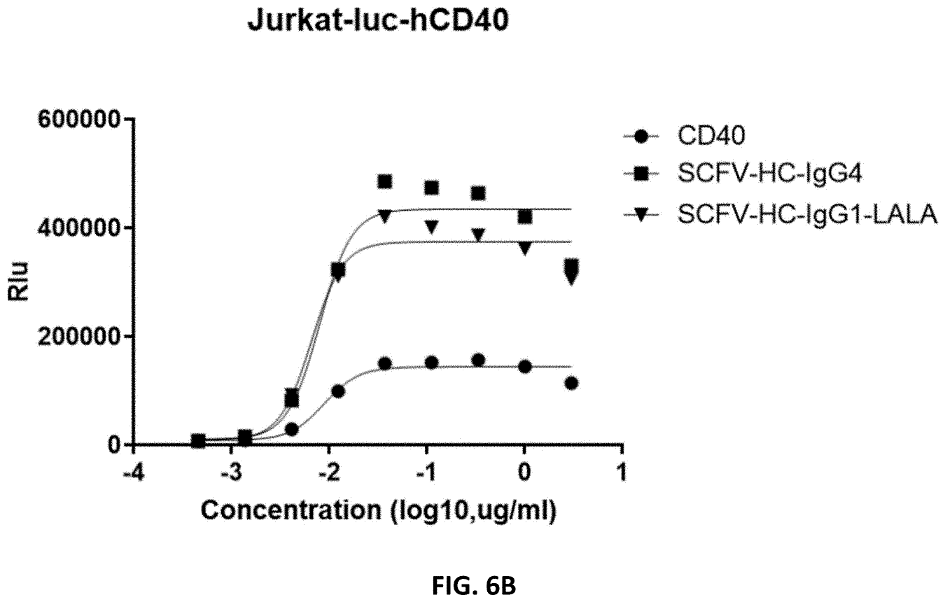

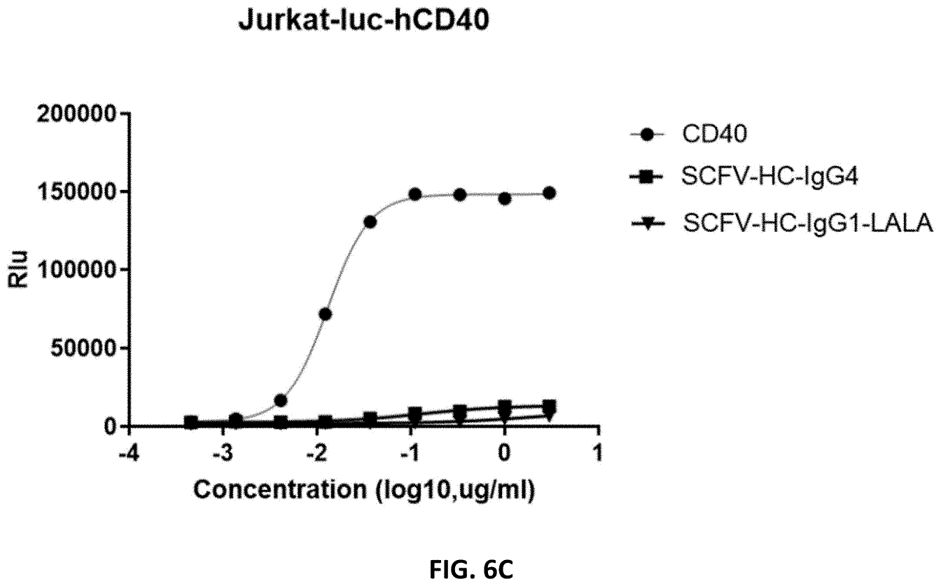

The experiment was performed to test whether subtypes of ScFV-HC-IgG, including ScFV-HC-IgG4 and ScFV-HC-IgG1-LALA, can activate reporter cells in the presence of basal cells expressing PD-1. The experiment was performed similarly to the procedures described in Example 4, except that the basal cells CHO-K1-hPD1 were replaced with Jurkat-hPD1. No Basal cells were used in FIG. 6A. As shown in FIG. 6B and Table 7, both the BsAbs ScFV-HC-IgG4 and ScFV-HC-IgG1-LALA, and the anti-CD40 monoclonal antibody 6A7-H4K2-IgG2 activated the reporter cells.

TABLE 7

| Antibody Name | EC50 Value (ug/ml) | R² value |
|---|---|---|
| CD40 | 0.0089 | 0.9612 |
| ScFV-HC-IgG4 | 0.0079 | 0.9473 |
| ScFV-HC-IgG4-LALA | 0.0065 | 0.9642 |

Reporter cell activation in the presence of CHO-K1-hFcγRIIB cells were also evaluated. As shown in FIG. 6C, neither of ScFV-HC-IgG4 and ScFV-HC-IgG1-LALA exhibited FCγRIIB receptor-mediated reporter cell activation, which was consistent with the result shown in FIG. 5. Thus, ScFV-HC-IgG4 and ScFV-HC-IgG1-LALA mainly relies on PD-1 expressing cells to activate CD40 pathway in APC cells.

### Example 7. In Vivo Testing of Bispecific Antibodies to Inhibit Tumor Growth

In order to test the bispecific antibodies in vivo and to predict the effects of these antibodies in human, a humanized CD40 mouse model was generated. The humanized CD40 mouse model was engineered to express a chimeric CD40 protein (SEQ ID NO: 97) wherein the extracellular region of the mouse CD40 protein was replaced with the corresponding human CD40 extracellular region. The amino acid residues 20-192 of mouse CD40 (SEQ ID NO: 96) were replaced by amino acid residues 20-192 of human CD40 (SEQ ID NO: 95). A double humanized CD40/PD-1 mouse model was also generated by crossing the CD40 humanized mice with PD-1 humanized mice. The humanized PD1 mouse model was engineered to express a chimeric PD1 protein (SEQ ID NO: 39) wherein the extracellular region of the mouse PD1 protein was replaced with the corresponding human PD1 extracellular region. The amino acid residues 31-141 of mouse PD1 (SEQ ID NO: 38) were replaced by amino acid residues 31-141 of human PD1 (SEQ ID NO: 37).

The humanized mouse models (e.g., B-hCD40 mice, or double humanized CD40/PD-1 mice (B-hPD-1/hCD40 mice) provide a new tool for testing new therapeutic treatments in a clinical setting by significantly decreasing the difference between clinical outcome in human and in ordinary mice expressing mouse CD40 or PD-1. A detailed description regarding humanized CD40, humanized PD-1, or double humanized CD40/PD-1 mouse models can be found in PCT/CN2018/091845 and PCT/CN2017/090320; each of which is incorporated herein by reference in its entirety.

### In Vivo Results for BsABs Against Colon Cancer

The bispecific antibodies ScFV-HC-IgG4 and ScFV-HC-IgG1-LALA were tested for their effect on tumor growth in vivo in a model of colon carcinoma. MC-38 cancer tumor cells (colon adenocarcinoma cell) were injected subcutaneously in CD40 humanized B-hCD40 mice. When the tumors in the mice reached a volume of 100-150 mm³, the mice were randomly placed into different groups based on the volume of the tumor.

In each group, B-hCD40 mice were injected with physiological saline (PS) (G1), 4 mg/kg ScFV-HC-IgG4 (G2), or 4 mg/kg ScFV-HC-IgG1-LALA (G3) by intraperitoneal (i.p.) administration. The frequency of administration was twice a week (4 administrations in total).

The injected volume was calculated based on the weight of the mouse at 4 mg/kg. The length of the long axis and the short axis of the tumor were measured and the volume of the tumor was calculated as 0.5×(long axis)×(short axis)². The weight of the mice was also measured before the injection, when the mice were placed into different groups (before the first antibody injection), twice a week during the antibody injection period, and before euthanization.

The tumor growth inhibition percentage (TGI%) was calculated using the following formula: TGI (%)=[1−(Ti−T0)/(Vi−V0)]×100. Ti is the average tumor volume in the treatment group on day i. T0 is the average tumor volume in the treatment group on day zero. Vi is the average tumor volume in the control group on day i. V0 is the average tumor volume in the control group on day zero.

T-test was performed for statistical analysis. A TGI% higher than 60% indicates clear suppression of tumor growth. P<0.05 is a threshold to indicate significant difference.

TABLE 8

| | | | | | Total No. |
|---|---|---|---|---|---|
| | | Dosage | | Fre- | of admin- |
| Group | Antibodies | (mg/kg) | Route | quency | istration |
| G1 | PS (control) | — | i.p. | BIW | 4 |
| G2 | ScFV-HC-IgG4 | 4 mg/kg | i.p. | BIW | 4 |
| G3 | ScFV-HC-IgG1- | 4 mg/kg | i.p. | BIW | 4 |
| | LALA | | | | |

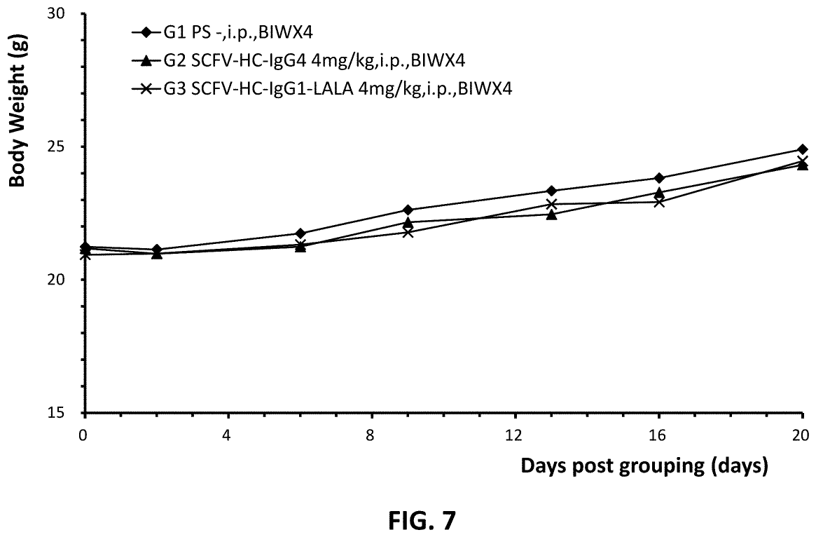

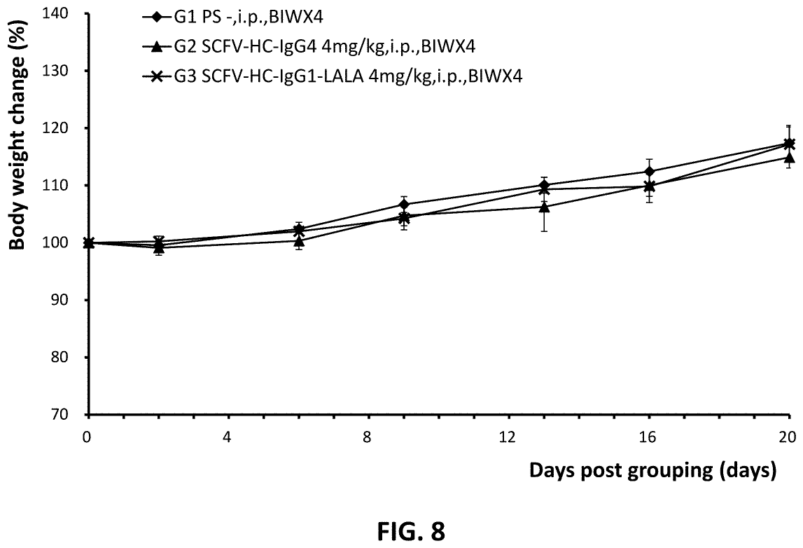

The weight of the mice was monitored during the entire treatment period. The average weight of mice in different groups all increased to different extents (FIG. 7, and FIG. 8). No obvious difference in weight was observed among different groups at the end of the treatment periods. The results showed that ScFV-HC-IgG4 and ScFV-HC-IgG1-LALA were well tolerated and were not obviously toxic to the mice.

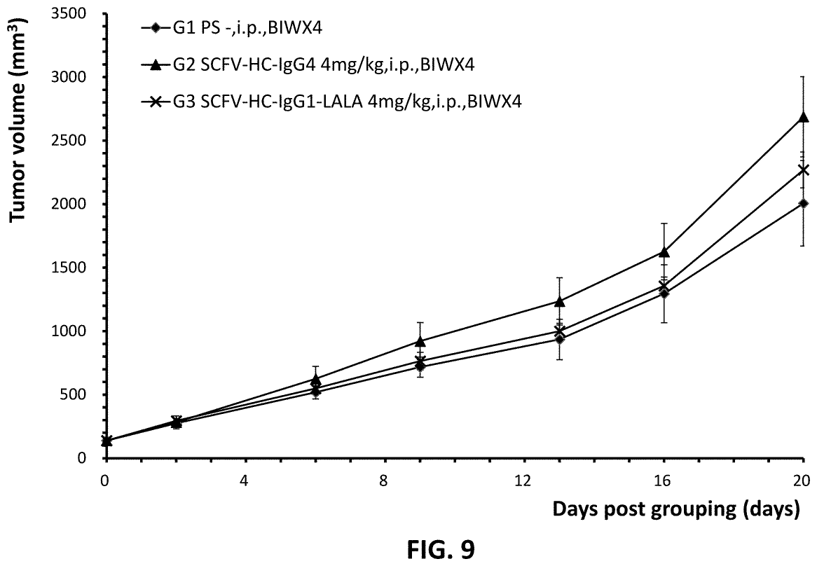

The tumor size in groups treated with ScFV-HC-IgG4 and ScFV-HC-IgG1-LALA is shown in FIG. 9. The TGI% on day 20 (20 days after grouping) was also calculated as shown in the table below.

TABLE 9

| | | Tumor volume (mm³) | | | | | | P value | |
|---|---|---|---|---|---|---|---|---|---|
| | | Day | Day | Day | Day | | | Body | Tumor |
| | | 0 | 6 | 13 | 20 | Survival | TGI_{TV} % | weight | Volume |
| Control | G1 | 139 ± 8 | 519 ± 52 | 935 ± 159 | 2007 ± 336 | 5/5 | n.a. | n.a. | n.a. |
| Treat | G2 | 139 ± 10 | 624 ± 99 | 1235 ± 186 | 2688 ± 316 | 5/5 | −36.5% | 0.626 | 0.178 |
| | G3 | 140 ± 9 | 549 ± 43 | 1001 ± 62 | 2270 ± 141 | 5/5 | −14.1% | 0.717 | 0.492 |

The results showed that bispecific antibodies ScFV-HC-IgG4 and ScFV-HC-IgG1-LALA did not inhibit tumor growth in the absence of cells expressing human PD-1.

### In Vivo Results for BsABs Against Melanoma

The bispecific antibodies ScFV-HC-IgG4 and ScFV-HC-IgG1-LALA were tested for their effect on tumor growth in vivo in a model of melanoma. B16F10 cells (melanoma cells) expressing human PD-L1 (B16F10-hPD-L1) were injected subcutaneously in double humanized CD40/PD-1 mice (B-hPD-1/hCD40 mice). When the tumors in the mice reached a volume of 100-150 mm³, the mice were randomly placed into different groups based on the volume of the tumor.

In each group, B-hPD-1/hCD40 mice were injected with physiological saline (PS) (G1), 3 mg/kg anti-PD1 monoclonal antibody 1A7-H2K3-IgG4 (G2), 3 mg/kg anti-CD40 monoclonal antibody 6A7-H4K2-IgG2 (G3), 4 mg/kg ScFV-HC-IgG1-LALA (G4), 4 mg/kg ScFV-HC-IgG4 (G5), combination of 3 mg/kg anti-PD1 monoclonal antibody 1A7-H2K3-IgG4 and 3 mg/kg anti-CD40 monoclonal antibody 6A7-H4K2-IgG2 (G6), or 4 mg/kg bispecific antibody PD-1-MOCK-ScFV-HC-IgG4 (G7) by intraperitoneal (i.p.) administration. The BsAb PD-1-MOCK-ScFV-HC-IgG4 has the same structure as ScFV-HC-IgG, but the scFv targets OX40, not CD40. The frequency of administration was twice a week (4 administrations in total).

The injected volume was calculated based on the weight of the mouse. The length of the long axis and the short axis of the tumor were measured and the volume of the tumor was calculated as 0.5×(long axis)×(short axis). The weight of the mice was also measured before the injection, when the mice were placed into different groups (before the first antibody injection), twice a week during the antibody injection period, and before euthanization. T-test was performed for statistical analysis. A TGI% higher than 60% indicates clear suppression of tumor growth. P<0.05 is a threshold to indicate significant difference.

TABLE 10

| | | | | | Total No. |
|---|---|---|---|---|---|
| | | Dosage | | Fre- | of admin- |
| Group | Antibodies | (mg/kg) | Route | quency | istration |
| G1 | PS (control) | — | i.p. | BIW | 4 |
| G2 | PD-1 | 3 mg/kg | i.p. | BIW | 4 |
| G3 | CD40 | 3 mg/kg | i.p. | BIW | 4 |
| G4 | ScFV-HC-IgG1- | 4 mg/kg | i.p. | BIW | 4 |
| | LALA | | | | |
| G5 | ScFV-HC-IgG4 | 4 mg/kg | i.p. | BIW | 4 |
| G6 | PD-1 + CD40 | 3 + 3 | i.p. | BIW | 4 |
| | | mg/kg | | | |
| G7 | PD-1-MOCK-ScFV- | 4 mg/kg | i.p. | BIW | 4 |
| | HC-IgG4 | | | | |

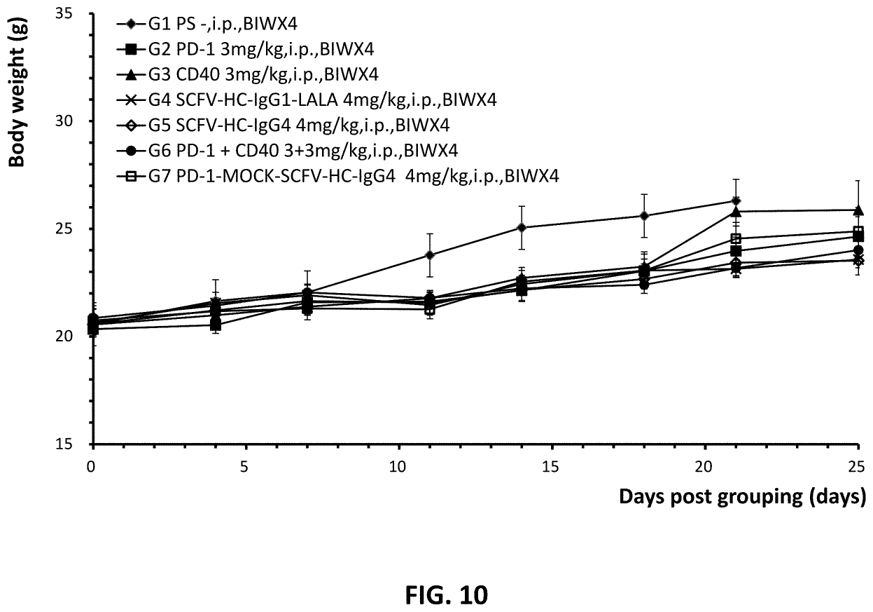

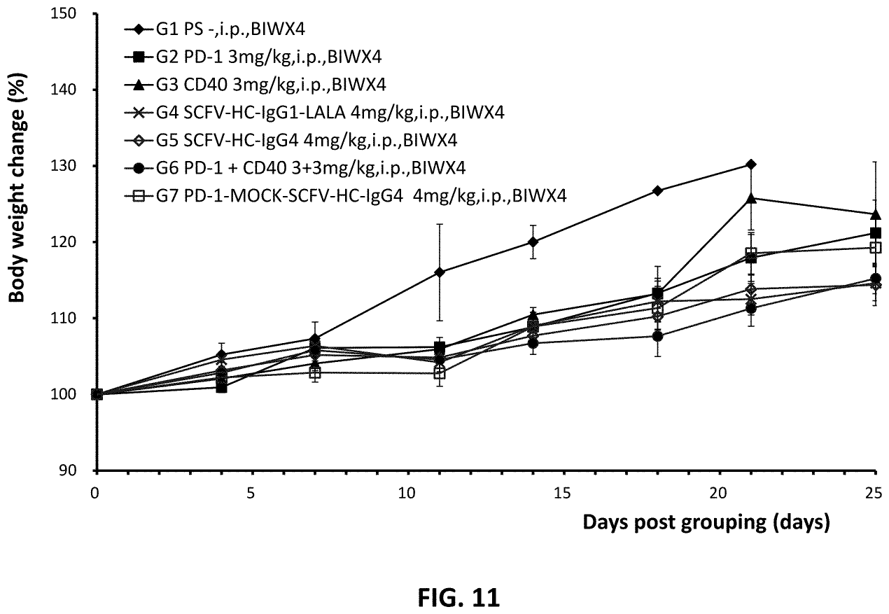

The weight of the mice was monitored during the entire treatment period. The average weight of mice in different groups all increased to different extents (FIG. 10, and FIG. 11). All the mice gained weight among different groups at the end of the treatment period.

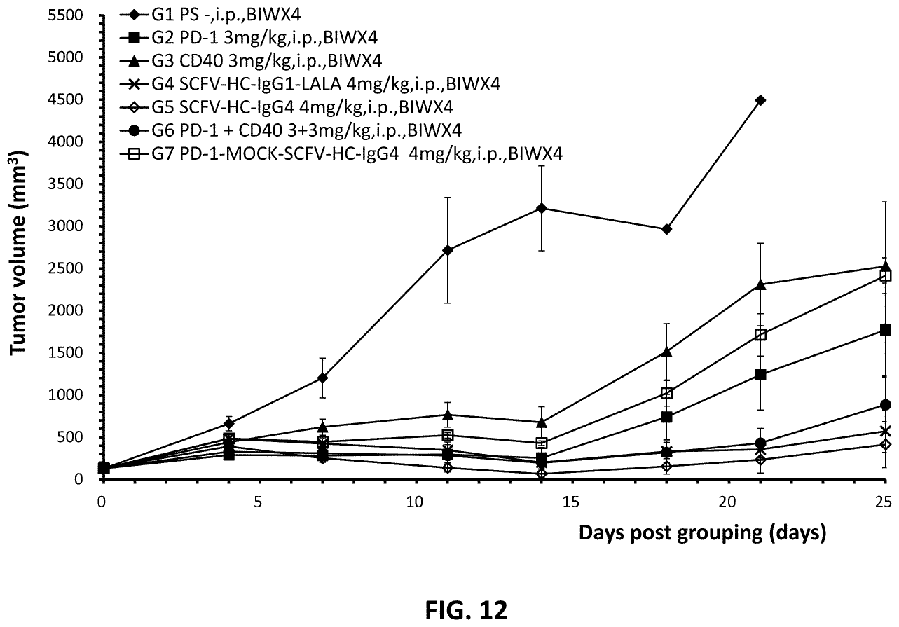

The tumor size in groups treated with the antibodies is shown in FIG. 12. The TGI% on day 14 and day 21 (14 or 21 days after grouping) was also calculated as shown in the table below. P value is based on the data on day 21. FIG. 12 also has data for day 25.

TABLE 11

| | | Tumor volume | | | | | | | P value for | |
|---|---|---|---|---|---|---|---|---|---|---|
| | | (mm³) | | | | Survival | TGI_{TV} | TGI_{TV} | Day 21 | |
| | | Day | Day | Day | Day | on Day | % on Day | % on Day | Body | Tumor |
| | | 0 | 7 | 14 | 21 | 21 | 14 | 21 | weight | Volume |
| Control | G1 | 132 ± 4 | 1203 ± 236 | 3213 ± 503 | 4491 | 1/7* | n.a. | n.a. | n.a. | n.a. |
| Treat | G2 | 132 ± 5 | 284 ± 81 | 254 ± 126 | 1240 ± 417 | 7/7 | 96.0% | 74.6% | <0.007 | <0.001 |
| | G3 | 132 ± 5 | 621 ± 95 | 677 ± 186 | 2311 ± 488 | 7/7 | 82.3% | 50.0% | <0.021 | <0.001 |
| | G4 | 132 ± 7 | 425 ± 61 | 201 ± 78 | 355 ± 127 | 7/7 | 97.8% | 94.9% | <0.008 | <0.001 |
| | G5 | 132 ± 5 | 252 ± 43 | 67 ± 40 | 234 ± 157 | 7/7 | 102.1% | 97.6% | <0.006 | <0.001 |
| | G6 | 132 ± 7 | 310 ± 62 | 197 ± 70 | 431 ± 176 | 7/7 | 97.9% | 93.1% | <0.003 | <0.001 |
| | G7 | 132 ± 6 | 445 ± 38 | 430 ± 26 | 1714 ± 250 | 7/7 | 90.3% | 63.7% | <0.016 | <0.001 |

Note:
*Early euthanization was performed on six mice because the tumor size exceeded 3000 mm³ before Day 21.

The results showed that bispecific antibodies ScFV-HC-IgG4 and ScFV-HC-IgG1-LALA inhibited tumor growth with a higher TGI% than the tested monoclonal antibodies. In particular, ScFV-HC-IgG4 (G5) and ScFV-HC-IgG1-LALA (G4) at 4 mg/kg had similar TGI% as compared to the combined anti-PD1 and anti-CD40 antibodies (G6) at 6 mg/kg. The results also indicate that the anti-CD40 scFv domain has a synergistic effect on tumor suppression.

In addition, the TGI% of PD-1-MOCK-ScFV-HC-IgG4 (G7) was comparable to that of the anti-PD1 monoclonal antibody 1A7-H2K3-IgG4 (G2), but was much lower (e.g., on day 21) than those of ScFV-HC-IgG4 (G5) and ScFV-HC-IgG1-LALA (G4). This indicates that the tumor inhibition effect induced by ScFV-HC-IgG4 (G5) and ScFV-HC-IgG1-LALA (G4) were partially induced by bridging cells expressing PD-1 (e.g., T cells) and cells expressing CD40 (e.g., APC cells).

### In Vivo Results for BsABs Against Colon Cancer

The bispecific antibodies ScFV-HC-IgG4, ScFV-HC-IgG1-LALA, and Fab-ScFV-IgG4 were tested for their effect on tumor growth in vivo in a model of colon carcinoma. MC-38 cancer tumor cells (colon adenocarcinoma cell) expressing human PD-L1 (MC38-hPD-L1) were injected subcutaneously in double humanized CD40/PD-1 mice (B-hPD-1/hCD40 mice). When the tumors in the mice reached a volume of about 400 mm³, the mice were randomly placed into different groups (7 mice per group) based on the volume of the tumor.

In each group, B-hPD-1/hCD40 mice were injected with physiological saline (NC;G1), 1 mg/kg anti-PD1 monoclonal antibody 1A7-H2K3-IgG4 (G2), 1 mg/kg anti-CD40 monoclonal antibody 6A7-H4K2-IgG2 (G3), 1.35 mg/kg ScFV-HC-IgG4 (G4), 1 mg/kg Fab-ScFV-IgG4 (G5), or combination of 1 mg/kg anti-PD1 monoclonal antibody 1A7-H2K3-IgG4 and 1 mg/kg anti-CD40 monoclonal antibody 6A7-H4K2-IgG2 (Combo (PD-1+CD40); G6) by intraperitoneal (i.p.) administration. The frequency of administration was twice a week (5 administrations in total). Details are shown in the table below.

TABLE 12

| | | | | | Total No. |
|---|---|---|---|---|---|
| | | Dosage | | Fre- | of admin- |
| Group | Antibodies | (mg/kg) | Route | quency | istration |
| G1 | PS (NC) | — | i.p. | BIW | 5 |
| G2 | PD-1 | 1 mg/kg | i.p. | BIW | 5 |
| G3 | CD40 | 1 mg/kg | i.p. | BIW | 5 |
| G4 | ScFV-HC-IgG4 | 1.35 mg/kg | i.p. | BIW | 5 |
| G5 | Fab-ScFV-IgG4 | 1 mg/kg | i.p. | BIW | 5 |
| G6 | PD-1 + CD40 | 1 + 1 | i.p. | BIW | 5 |
| | | mg/kg | | | |

The injected volume was calculated based on the weight of the mouse. The length of the long axis and the short axis of the tumor were measured and the volume of the tumor was calculated as 0.5×(long axis)×(short axis).

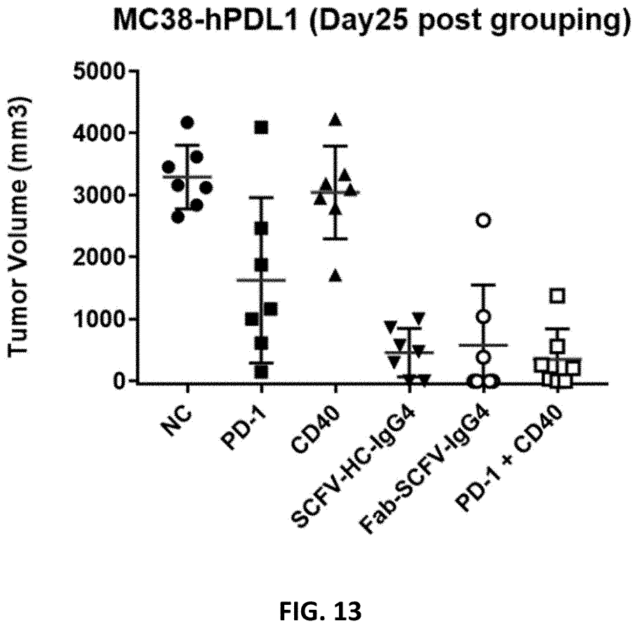

The tumor volumes of mice in the above groups on Day 25 post grouping are shown in FIG. 13. The results showed that bispecific antibodies ScFV-HC-IgG4 and Fab-ScFV-IgG4 inhibit tumor growth. In particular, ScFV-HC-IgG4 (G4; 1.35 mg/kg) and Fab-ScFV-IgG4 (G5; 1 mg/kg) had similar TGITV% as compared to the combined anti-PD1 and anti-CD40 antibodies (G6) at 2 mg/kg.

In a different experiment, ScFv-HC-IgG1-LALA and anti-PD-1 monoclonal antibody Keytruda® (pembrolizumab) (VH SEQ ID NO: 207; VL SEQ ID NO: 208) were also included.

In each group, B-hPD-1/hCD40 mice were injected with physiological saline (NC;G1), 1 mg/kg anti-PD1 monoclonal antibody 1A7-H2K3-IgG4 (G2), 1 mg/kg anti-CD40 monoclonal antibody 6A7-H4K2-IgG2 (G3), 1.35 mg/kg ScFV-HC-IgG4 (G4), 1 mg/kg ScFV-HC-IgG1-LALA (G5), combination of 1 mg/kg anti-PD1 monoclonal antibody 1A7-H2K3-IgG4 and 1 mg/kg anti-CD40 monoclonal antibody 6A7-H4K2-IgG2 (Combo (PD-1+CD40); G6) , or combination of 1 mg/kg anti-PD1 monoclonal antibody pembrolizumab and 1 mg/kg anti-CD40 monoclonal antibody 6A7-H4K2-IgG2 (Combo (pembrolizumab +CD40); G7) by intraperitoneal (i.p.) administration. The frequency of administration was twice a week (5 administrations in total). Details are shown in the table below.

TABLE 13

| | | | | | | Total No. |
|---|---|---|---|---|---|---|
| | | Dosage | | | Fre- | of admin- |
| Group | Antibodies | (mg/kg) | | Route | quency | istration |
| G1 | PS | — | | i.p. | BIW | 6 |
| G2 | PD-1 | 1 | mg/kg | i.p. | BIW | 6 |
| G3 | CD40 | 1 | mg/kg | i.p. | BIW | 6 |
| G4 | ScFV-HC-IgG4 | 1.35 | mg/kg | i.p. | BIW | 6 |
| G5 | ScFV-HC-IgG1- | 1.35 | mg/kg | i.p. | BIW | 6 |
| | LALA | | | | | |

| | | | | | |
|---|---|---|---|---|---|
| G6 | PD-1 + CD40 | 1 + 1 | i.p. | BIW | 6 |
| | | mg/kg | | | |
| G7 | pembrolizumab + | 1 + 1 | i.p. | BIW | 6 |
| | CD40 | mg/kg | | | |

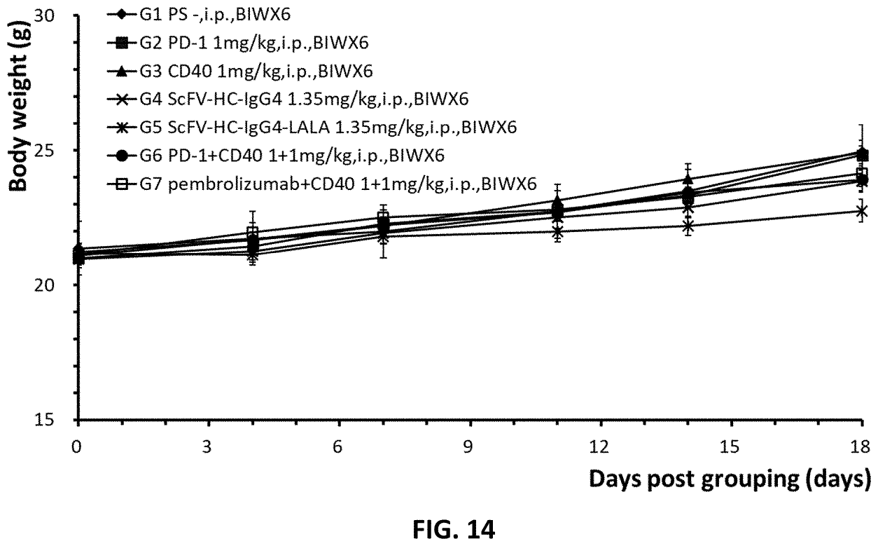

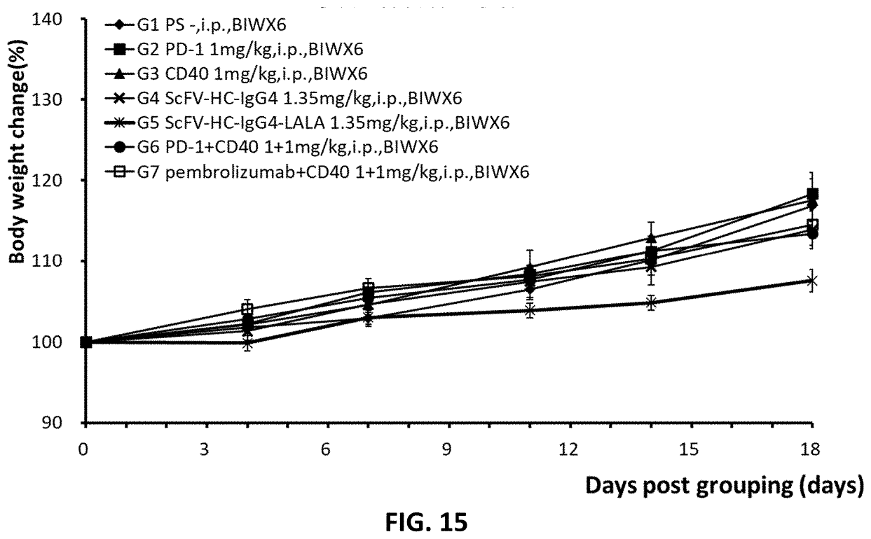

The weight of the mice was monitored during the entire treatment period. The average weight of mice in different groups all increased to different extents (FIG. 14, and FIG. 15). All the mice gained weight among different groups at the end of the treatment period.

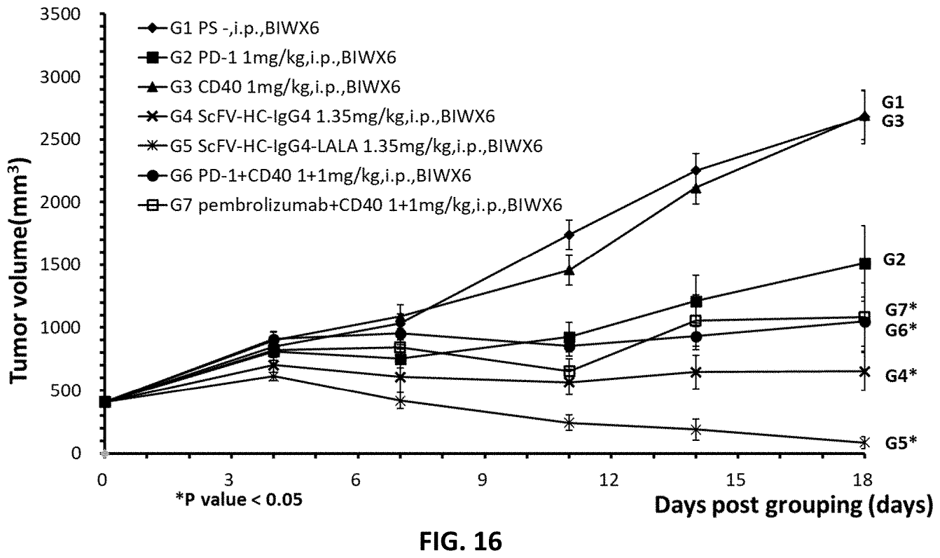

The tumor size in groups treated with the antibodies is shown in FIG. 16. The TGI% on day 18 and day 25 (18 days and 25 days after grouping) was also calculated as shown in the table below. As a large number of mice in the control group died, P value for Day 25 was not available. P value in the following table was calculated based on the data on day 18.

TABLE 14

| | | Tumor volume | | | | | | | | P value for | |
|---|---|---|---|---|---|---|---|---|---|---|---|
| | | (mm³) | | | | Survival | Survival | TGI_{TV} | TGI_{TV} | Day 18 | |
| | | Day | Day | Day | Day | on Day | on Day | % on Day | % on Day | Body | Tumor |
| | | 0 | 11 | 18 | 25 | 18 | 25 | 18 | 25 | weight | Volume |
| Control | G1 | 411 ± 3 | 1739 ± 117 | 2679 ± 214 | 3196 | 7/7 | 1/7 | n.a. | n.a. | n.a. | n.a. |
| Treat | G2 | 411 ± 6 | 926 ± 118 | 1512 ± 302 | 2073 ± 360 | 7/7 | 6/7 | 51.5% | 40.3% | 0.810 | 0.008 |
| | G3 | 411 ± 4 | 1458 ± 120 | 2692 ± 194 | n.a. | 7/7 | 0/7 | −0.5% | n.a. | 0.928 | 0.967 |
| | G4 | 411 ± 4 | 566 ± 99 | 651 ± 152 | 930 ± 334 | 7/7 | 7/7 | 89.4% | 81.4% | 0.077 | 5.40E−06 |
| | G5 | 411 ± 4 | 243 ± 63 | 86 ± 50 | 0 | 7/7 | 7/7 | 114.3% | 113.4% | 0.003 | 5.85E−08 |
| | G6 | 411 ± 4 | 854 ± 79 | 1048 ± 195 | 1561 ± 383 | 7/7 | 7/7 | 71.9% | 58.7% | 0.102 | 1.11E−04 |
| | G7 | 411 ± 5 | 656 ± 96 | 1084 ± 270 | 1326 ± 291 | 7/7 | 6/7 | 70.3% | 67.2% | 0.216 | 0.001 |

The results showed that ScFV-HC-IgG1-LALA (G5) inhibited tumor growth with a higher TGI% (e.g., on day 18) than that of ScFV-HC-IgG4 (G4). In addition, the combination of 1A7-H2K3-IgG4 and 6A7-H4K2-IgG2 (G6) exhibited a comparable tumor inhibition effect as compared to that of combination of pembrolizumab and 6A7-H4K2-IgG2 (G7).

### Example 8. In Vivo Testing of Bispecific Antibody Toxicity

The bispecific antibodies ScFV-HC-IgG4 was tested for its toxicity in vivo in a model of colon carcinoma. MC-38 cancer tumor cells (colon adenocarcinoma cell) expressing human PD-L1 (MC38-hPD-L1) were injected subcutaneously in double humanized CD40/PD-1 mice (B-hPD-1/hCD40 mice). When the tumors in the mice reached a volume of 100-150 mm³, the mice were randomly placed into different groups (3 mice per group) based on the volume of the tumor.

In each group, B-hPD-1/hCD40 mice were injected with phosphate-buffered saline (PBS) (G1), 20 mg/kg Selicrelumab (heavy chain SEQ ID NO: 144; light chain SEQ ID NO: 145) (G2), 20 mg/kg anti-CD40 monoclonal antibody 6A7-H4K2-IgG2 (G3), 20 mg/kg anti-PD1 monoclonal antibody 1A7-H2K3-IgG4 (G4), 26 mg/kg ScFV-HC-IgG4 (G5), or combination of 20 mg/kg anti-PD1 monoclonal antibody 1A7-H2K3-IgG4 and 20 mg/kg anti-CD40 monoclonal antibody 6A7-H4K2-IgG2 (Combo; G6) by intraperitoneal (i.p.) administration. The frequency of administration was three times a week (3 administrations in total). Details are shown in the table below.

TABLE 15

| | | | | | Total No. |
|---|---|---|---|---|---|
| | | Dosage | | Fre- | of admin- |
| Group | Antibodies | (mg/kg) | Route | quency | istration |
| G1 | PBS (control) | — | i.p. | Day 0, | 3 |
| | | | | Day 3, | |
| | | | | Day 6 | |
| G2 | Selicrelumab | 20 mg/kg | i.p. | Day 0, | 3 |
| | | | | Day 3, | |
| | | | | Day 6 | |
| G3 | CD40 | 20 mg/kg | i.p. | Day 0, | 3 |
| | | | | Day 3, | |
| | | | | Day 6 | |
| G4 | PD-1 | 20 mg/kg | i.p. | Day 0, | 3 |
| | | | | Day 3, | |
| | | | | Day 6 | |
| G5 | ScFV-HC-IgG4 | 26 mg/kg | i.p. | Day 0, | 3 |
| | | | | Day 3, | |
| | | | | Day 6 | |
| G6 | PD-1 + CD40 | 20 + 20 | i.p. | Day 0, | 3 |
| | | mg/kg | | Day 3, | |
| | | | | Day 6 | |

### Blood Biochemical Test

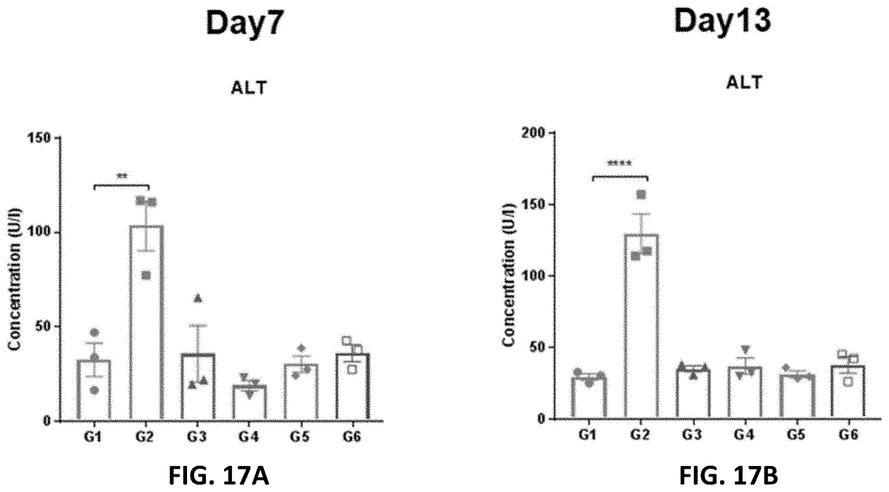

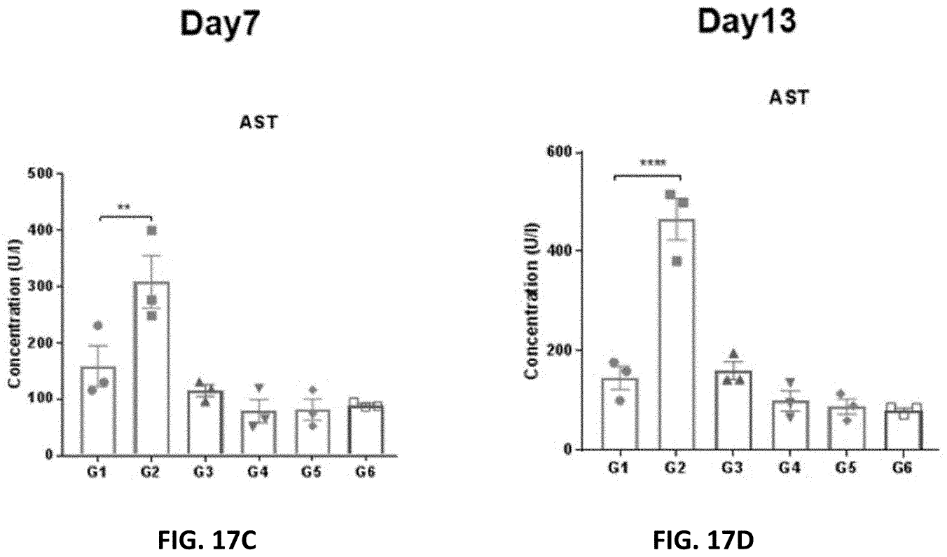

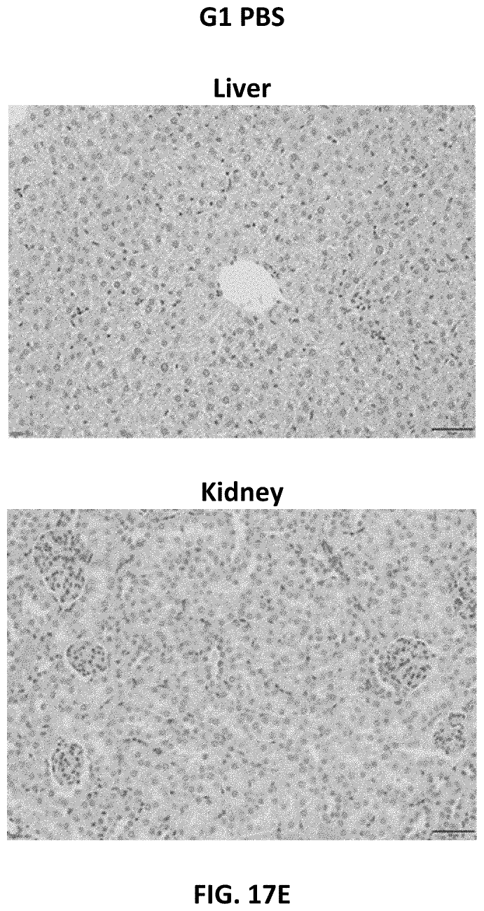

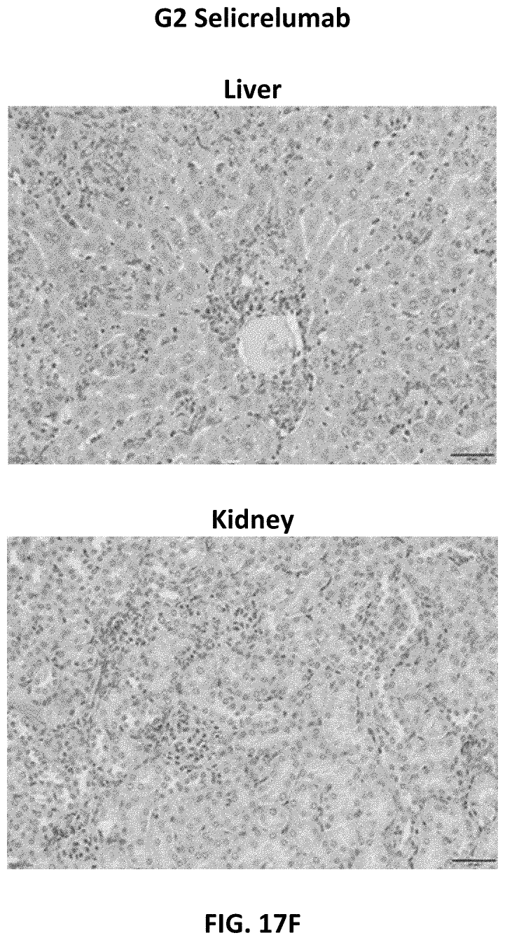

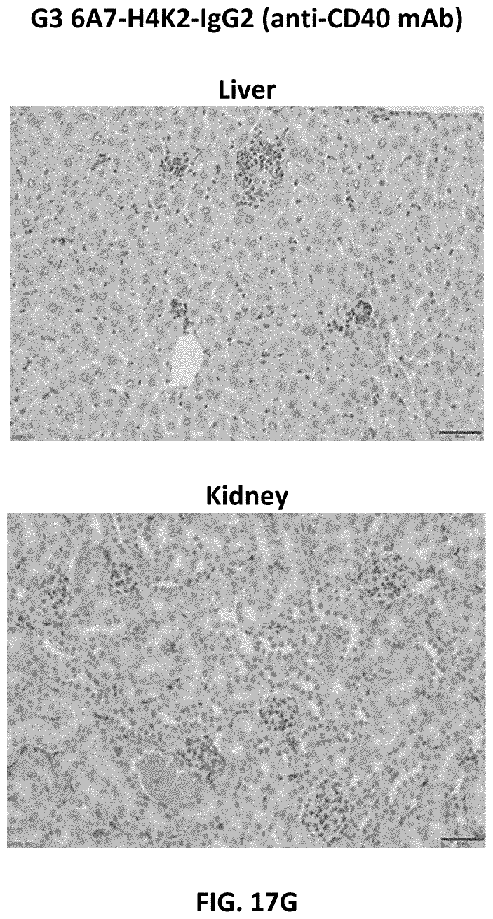

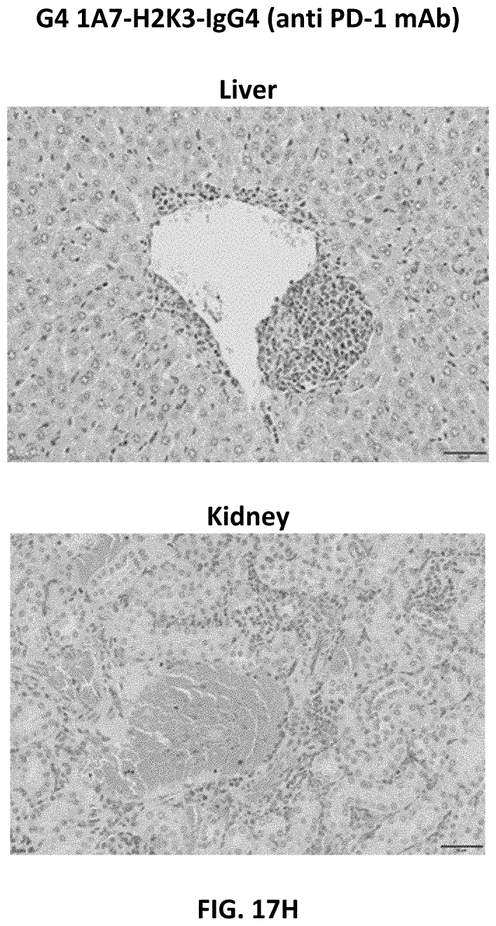

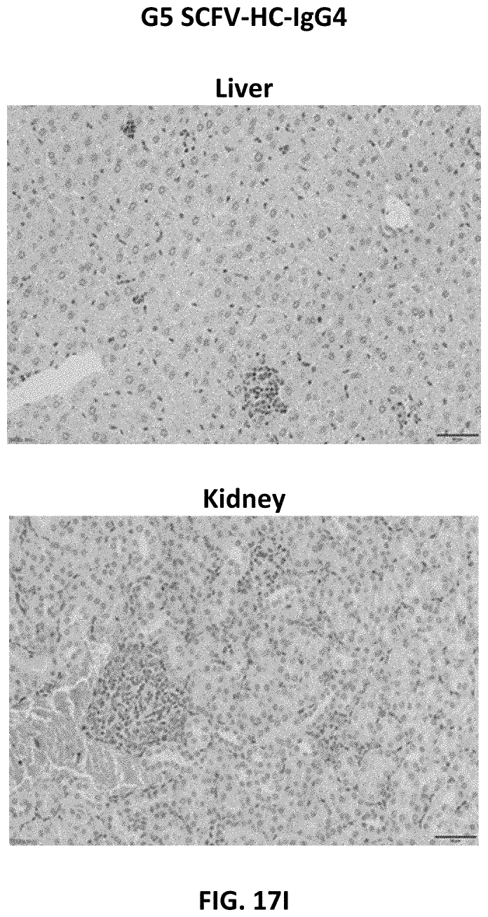

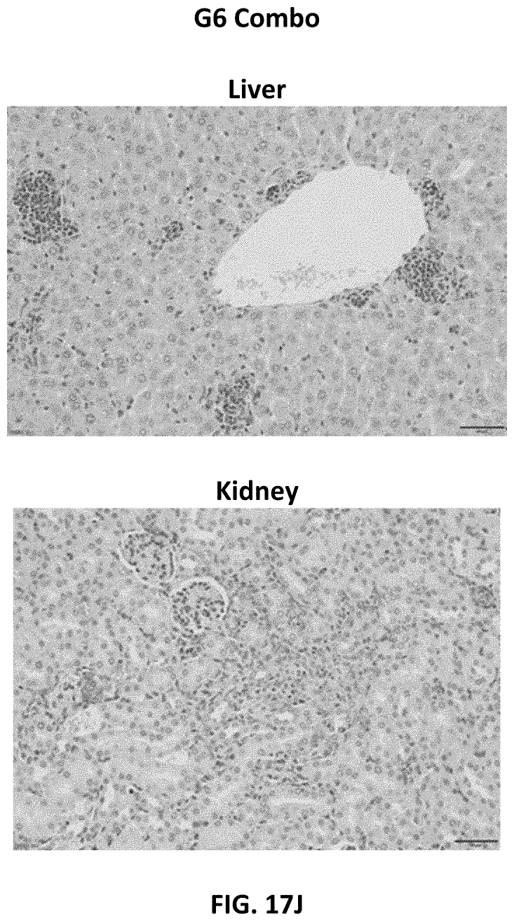

On day 7 and day 13 after grouping, mouse blood was withdrawn to test alanine aminotransferase (ALT) and aspartate aminotransferase (AST) levels. As shown in FIGS. 17A-17D, mice treated with Selicrelumab (G2) exhibited highest level of blood ALT and AST levels as compared to mice in other groups. In addition, mice treated with ScFV-HC-IgG4 (G5) showed similar ALT and AST levels as compared to mice treated with monoclonal antibodies (G3 and G4) or combination thereof (G6).

### Histopathological Examination of Liver

On day 13 after grouping, the mouse liver was isolated and examined under microscope. There were no obvious abnormal changes in the liver of mice in group G1, and G5. Chronic inflammation, e.g., fibroblast proliferation with moderate degree of lesions, was observed in all three mice in group G2. In group G3, all three mice showed focal infiltration of interstitial inflammatory cells in the liver, with slight degree of lesions. In group G4, one mouse showed focal infiltration of perivascular inflammatory cell in the liver, with slight degree of lesions. In Group G7, all three mice showed focal infiltration of interstitial inflammatory cells in the liver. The degree of lesions was slight in one mouse and mild in two mice.

A detailed liver lesion degree is shown in the table below. The degree of lesion was determined by liver interstitial/perivascular inflammatory cell infiltration or chronic liver inflammation (mainly fibroblast proliferation). Representative histological section images of mouse liver and kidney in each group are shown in FIGS. 17E-17J.

TABLE 16

| Degree of lesions | G1 | G2 | G3 | G4 | G5 | G6 |
|---|---|---|---|---|---|---|
| NVL* | 3 | 0 | 0 | 2 | 3 | 0 |
| Slight (+) | 0 | 0 | 3 | 1 | 0 | 1 |
| Mile (++) | 0 | 0 | 0 | 0 | 0 | 2 |
| Moderate (+++) | 0 | 3 | 0 | 0 | 0 | 0 |
| Severe (++++) | 0 | 0 | 0 | 0 | 0 | 0 |

*non-visible lesion

### Example 9. Purification and Reporter Cell Activation by PD1-C40-6A7-FV3A

### Preparation of PD1-C40-6A7-FV3A

As shown in FIG. 18A, the BsAb PD1-C40-6A7-FV3A has an anti-CD40 scFv fused to each of the heavy chain CH3 domain of an anti-PD-1 monoclonal antibody. Specifically, the anti-CD40 scFv was fused to the heavy chain CH3 domain at a region from position 358 to position 362 (according to EU numbering) of the anti-PD-1 monoclonal antibody. The fused heavy chain sequence of PD1-C40-6A7-FV3A is set forth in SEQ ID NO: 161. The light chain sequence of PD1-C40-6A7-FV3A is identical to the light chain of its parent antibody PD1-1A7-H2K3-IgG4, which is set forth in SEQ ID NO: 141. Sequences are derived from the anti-PD-1 antibody 1A7 and the anti-CD40 antibody 6A7.

The purified PD1-C40-6A7-FV3A was detected by non-reducing gel electrophoresis (6% separation gel) as shown in FIG. 18B. A single band was detected on the gel (lane 4), indicating that PD1-C40-6A7-FV3A was expressed with high purity. In addition, the concentration of PD1-C40-6A7-FV3A was determined at 84 μg/ml.

Binding Affinity to hCD40

The binding affinity to human CD4 (hCD40) was determined by Biacore systems. Results of PD1-C40-6A7-FV3A and its parent monoclonal antibody 6A7-H4K2-IgG2 are summarized in the table below.

TABLE 17

| Sample | analysis | ka (1/Ms) | kd (1/s) | KD (M) |
|---|---|---|---|---|
| PD1-C40-6A7- | hCD40 | 6.08E+05 | 2.55E−04 | 4.19E−10 |
| FV3A | | | | |
| C40 (6A7-H4K2- | hCD40 | 1.05E+05 | 3.22E−04 | 3.06E−09 |
| IgG2) | | | | |

The result showed that PD1-C40-6A7-FV3A had a binding affinity that was comparable to the parent monoclonal antibody.

### Fc Receptor-Mediated Reporter Cell Activation Assay

The experiment was performed to test whether PD1-C40-6A7-FV3A can activate reporter cells via the Fc receptor (e.g., FCγRIIB) Similar experimental procedures were carried out as described in Example 5.

As shown in FIG. 18C, when CHO-K1-hFcγRIIB cells were present, both Fab-ScFV-IgG4 and the anti-CD40 monoclonal antibody 6A7-H4K2-IgG2 activated the reporter cells. However, PD1-C40-6A7-FV3A did not exhibit FCγRIIB receptor-mediated reporter cell activation.

The results showed that when the anti-CD40 scFv was fused to the heavy chain CH3 domain of the anti-PD-1 antibody, Fc receptor (e.g., FCγRIIB) cannot activate CD40 pathway through Fc receptor (e.g., FCγRIIB) mediated clustering. Because PD1-C40-6A7-FV3A did not exhibit Fc receptor-mediated reporter cell activation, PD1-C40-6A7-FV3A can effectively reduce toxicity in tissues, particularly tissues expressing high level of FCγRIIB, e.g., liver tissue.

### T Cell/APC Cell Bridging Effect

The experiment was performed to test whether PD1-C40-6A7-FV3A can bridge the T cells and APC cells using reporter cells. Similar experimental procedures were carried out as described in Example 4.

As shown in FIG. 18D, when CHO-K1-hPD1 cells were present, both Fab-ScFV-IgG4 and PD1-C40-6A7-FV3A activated the reporter cells in trans. By contrast, combination of the monoclonal antibodies did not activate the reporter cells.

The results also indicates that activation of the CD40 pathway by PD1-C40-6A7-FV3A depends on its bridging effect with PD-1 expressing cells. As immune cells expressing PD-1 will be recruited to the tumor microenvironment and PD-1 expression is usually up-regulated in the tumor microenvironment, this mechanism can also limit immune activation largely to tumor microenvironment and reduce side effects, e.g., toxicity in liver. In addition, the tumor draining lymph nodes has both antigen presenting cells and T cells expressing PD-1. The bispecific antibodies can effectively increase immune response in the tumor draining lymph nodes. In the meantime, it can suppress suppressor activity of regulatory T cells (Tregs), thereby inhibiting systematic tolerance.

### Binding Affinity to FcRn

The binding affinity to neonatal Fc receptor (FcRn) was determined by Biacore® systems. Results of PD1-C40-6A7-FV3A and an anti-PD-1 monoclonal antibody 1A7 are summarized in the table below.

TABLE 18

| Capture 1 Solution | Analyte 1 Solution | KD (M) |
|---|---|---|
| PD1-C40-6A7-FV3A | FcRn | 1.68E−06 |
| PD-1 (1A7-H2K3- | FcRn | 2.21E−06 |
| IgG4) | | |

The results showed that PD1-C40-6A7-FV3A had a binding affinity to FcRn that was comparable to the anti-PD-1 antibody (1A7-H2K3-IgG4), indicating that fusion of an anti-CD40 scFv at a region from position 358 to position 362 (according to EU numbering) of an anti-PD-1 antibody's heavy chain CH3 domain did not affect FcRn binding.

### Example 10. Comparison of Efficacy between ScFV-HC-IgG1-LALA and Anti-PD-1 Monoclonal Antibodies on the Market

At present, about ten anti-PD-1 monoclonal antibodies have been approved by the U.S. Food and Drug Administration (FDA) and the National Medical Products Administration (NMPA) in China. The approved anti-PD-1 monoclonal antibodies include Keytruda® (pembrolizumab) developed by Merck & Co.; Opdivo® (nivolumab, VH SEQ ID NO: 209; VL SEQ ID NO: 210) developed by Bristol Myers Squibb (BMS); Libtayo® (cemiplimab, VH SEQ ID NO: 185; VL SEQ ID NO: 186) jointly developed by Sanofi S.A. and Regeneron Pharmaceuticals, Inc.; Tyvyt® (sintilimab, VH SEQ ID NO: 183; VL SEQ ID NO: 184) developed by Innovent Biologics, Inc.; Tislelizumab (BGB-A317, VH SEQ ID NO: 187; VL SEQ ID NO: 188) developed by BeiGene; and Toripalimab (VH SEQ ID NO: 181; VL SEQ ID NO: 182) developed by Junshi Biosciences. In addition, there are multiple anti-PD-1 monoclonal antibodies in clinical and preclinical stages. In this experiment, in vivo drug efficacy of the bispecific antibody ScFV-HC-IgG1-LALA were compared against the efficacy of these approved anti-PD-1 monoclonal antibodies.

The experiment was performed as follows. Five anti-PD-1 antibodies pembrolizumab, cemiplimab, sintilimab, tislelizumab, toripalimab, and the bispecific antibody ScFV-HC-IgG1-LALA were tested for their inhibitory effect on tumor growth in vivo in a mouse melanoma model. Specifically, B16F10 cells (melanoma cells) expressing human PD-L1 (B16F10-hPD-L1) were injected subcutaneously in double humanized CD40/PD-1 mice (B-hPD-1/hCD40 mice). When the tumors in the mice reached a volume of about 100-150 mm³, the mice were randomly placed into different groups (7 mice per group) based on the tumor volume. In the control group, the B-hPD-1/hCD40 mice were injected with PBS (G1). In the treatment groups (G2-G7), the B-hPD-1/hCD40 mice were injected with 3 mg/kg pembrolizumab (G2), 3 mg/kg cemiplimab (G3), 3 mg/kg sintilimab (G4), 3 mg/kg tislelizumab (G5), 3 mg/kg toripalimab (G6), or 4 mg/kg of the bispecific antibody ScFV-HC-IgG1-LALA (G7) by intraperitoneal (i.p.) administration. The frequency of administration was twice a week (4 administrations in total). The tumor volume was measured twice a week and body weight of the mice was recorded as well. Euthanasia was performed when tumor volume of a mouse reached 3000 mm³.

TABLE 19

| | | | | | Total No. |
|---|---|---|---|---|---|
| | | Dosage | | Fre- | of admin- |
| Group | Antibodies | (mg/kg) | Route | quency | istration |
| G1 | PBS (control) | — | i.p. | BIW | 4 |
| G2 | Pembrolizumab-IgG4 | 3 mg/kg | i.p. | BIW | 4 |
| G3 | Cemiplimab-IgG4 | 3 mg/kg | i.p. | BIW | 4 |
| G4 | Sintilimab-IgG4 | 3 mg/kg | i.p. | BIW | 4 |
| G5 | Tislelizumab-IgG4 | 3 mg/kg | i.p. | BIW | 4 |
| G6 | Toripalimab-IgG4 | 3 mg/kg | i.p. | BIW | 4 |
| G7 | ScFV-HC-IgG1-LALA | 4 mg/kg | i.p. | BIW | 4 |

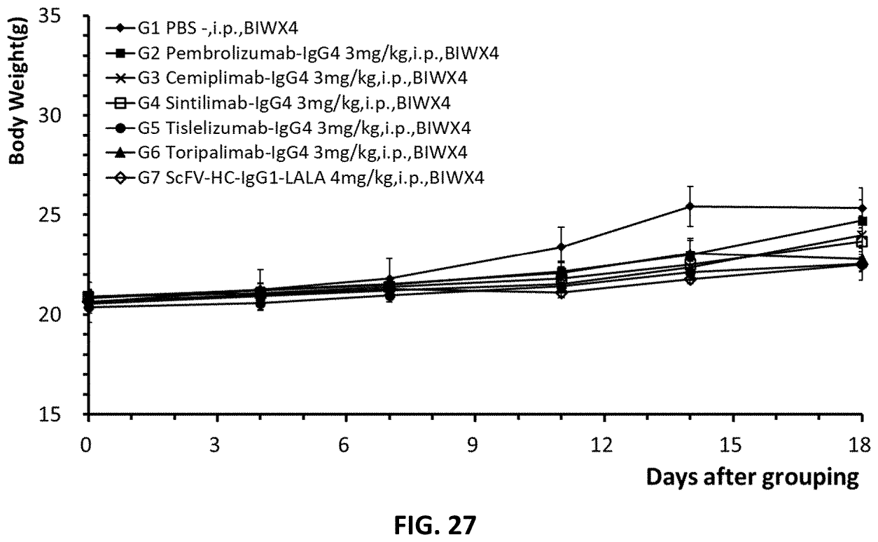

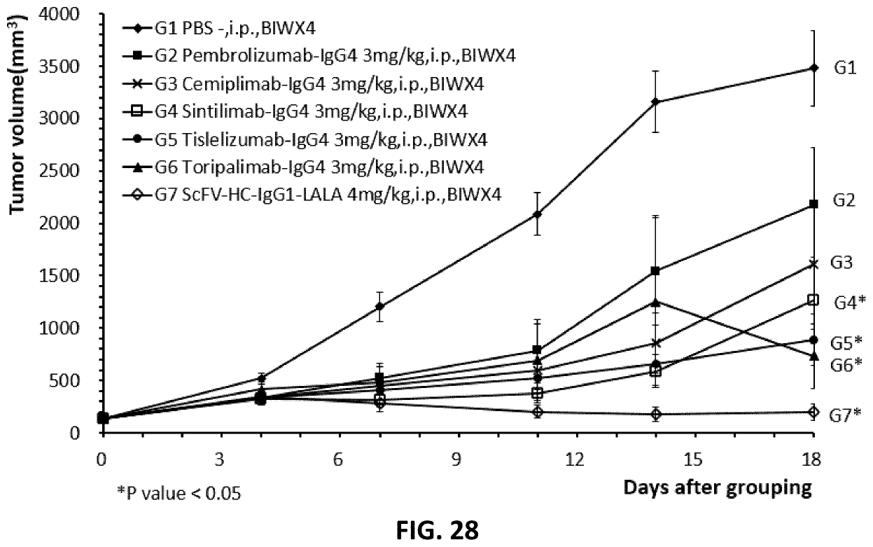

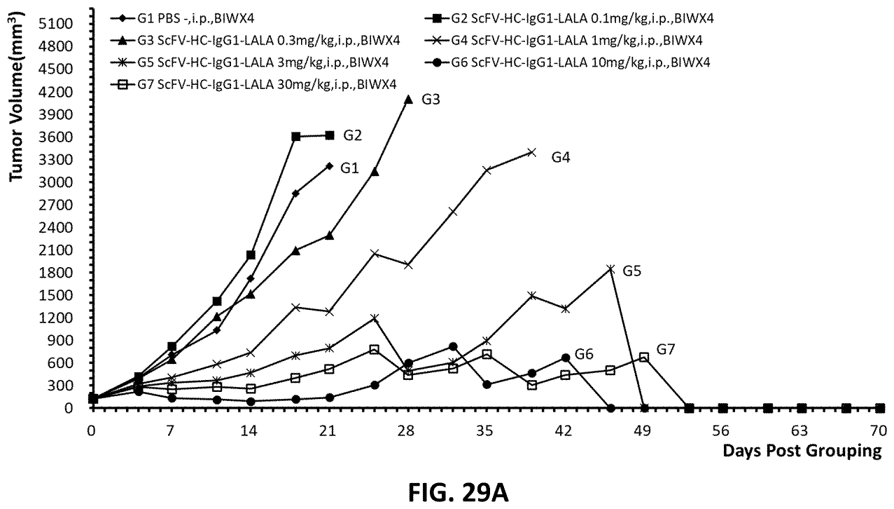

The experimental results showed that at the dosage and frequency of administration, the treatment group mice tolerated all the anti-PD-1 monoclonal antibodies and ScFV-HC-IgG1-LALA well. As shown in FIG. 27, there was no significant difference of the average mouse body weight in the entire experimental period. However, with respect to the tumor volume (FIG. 28), the bispecific antibody ScFV-HC-IgG1-LALA (G7) showed the highest efficacy (TGI_{TV}%).

TABLE 20

| | | Tumor volume | | | | | | | | Tumor | P value on | |
|---|---|---|---|---|---|---|---|---|---|---|---|---|
| | | (mm³) | | | | Survival | Survival | TGI_{TV} | TGI_{TV} | free | Day 18 | |
| | | Day | Day | Day | Day | on Day | on Day | % on Day | % on Day | on day | Body | Tumor |
| | | 0 | 11 | 14 | 18 | 14 | 18 | 14 | 18 | 18 | weight | Volume |
| Control | G1 | 132 ± 5 | 2087 ± 203 | 3159 ± 290 | 3480 ± 363 | 7/7 | 3/7 | n.a. | n.a. | 0 | n.a. | n.a. |
| Treat | G2 | 132 ± 5 | 784 ± 251 | 1543 ± 514 | 2179 ± 545 | 7/7 | 6/7 | 53.4% | 38.9% | 1 | 0.699 | 0.162 |
| | G3 | 132 ± 6 | 592 ± 175 | 859 ± 285 | 1606 ± 615 | 7/7 | 7/7 | 76.0% | 56.0% | 0 | 0.398 | 0.097 |
| | G4 | 132 ± 6 | 381 ± 99 | 590 ± 154 | 1084 ± 410 | 7/7 | 7/7 | 84.9% | 66.1% | 1 | 0.072 | 0.012 |
| | G5 | 132 ± 6 | 520 ± 138 | 653 ± 196 | 884 ± 245 | 7/7 | 7/7 | 82.8% | 77.6% | 0 | 0.059 | 3.85E−04 |
| | G6 | 132 ± 8 | 694 ± 391 | 1253 ± 818 | 734 ± 308 | 7/7 | 6/7 | 63.0% | 82.0% | 1 | 0.041 | 0.001 |
| | G7 | 132 ± 9 | 202 ± 65 | 176 ± 67 | 198 ± 83 | 7/7 | 7/7 | 98.6% | 98.0% | 2 | 0.001 | 1.20E−06 |

### Example 11. In Vivo Efficacy and Toxicity of ScFV-HC-IgG1-LALA

The bispecific antibodies ScFV-HC-IgG1-LALA was tested for its toxicity and efficacy in vivo in multiple tumor models. For example, B16F10-hPD-L1 cells were injected subcutaneously in double humanized CD40/PD-1 mice (B-hPD-1/hCD40 mice). When the tumors in the mice reached a volume of 100-150 mm³, the mice were randomly placed into different groups (7 mice per group) based on the tumor volume.

In control groups G1, the B-hPD-1/hCD40 mice were injected with physiological saline (PS). In treatment groups G2-G7, the B-hPD-1/hCD40 mice were injected with 0.1-30 mg/kg (i.e., 0.1 mg/kg, 0.3 mg/kg, 1 mg/kg, 3 mg/kg, 10 mg/kg, or 30 mg/kg) of the bispecific antibody ScFV-HC-IgG1-LALA by intraperitoneal (i.p.) administration. The frequency of administration was twice a week (4 administrations in total). The tumor volume was measured twice a week and body weight of the mice was recorded as well. Euthanasia was performed when tumor volume of a mouse reached 3000 mm³. The experiment was terminated on Day 70 post grouping.

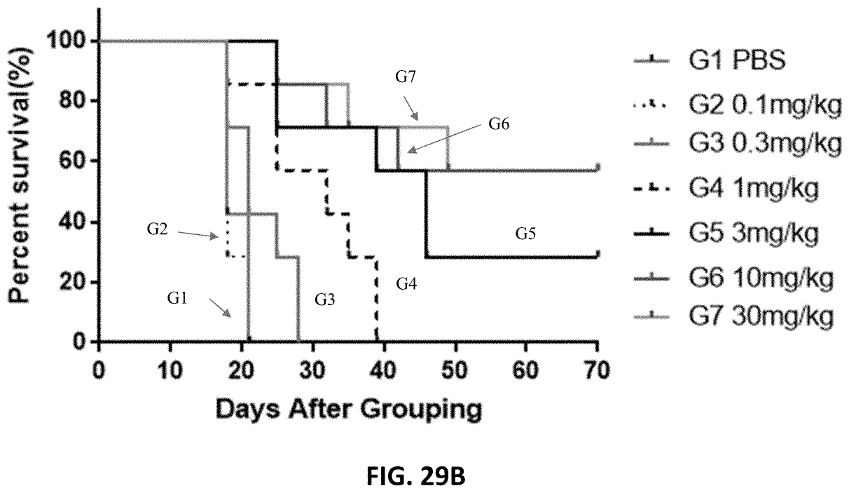

Similar to the previous results, the experimental results showed that under the dose levels and frequency as described above, the mice in the treatment groups tolerated different doses of ScFV-HC-IgG1-LALA very well, and there was no significant difference in the average of mouse body weight in the entire experimental period. However, with respect to the tumor volume (FIG. 29A) and mouse survival (FIG. 29B), the TGI_{TV}% showed an increasing trend, i.e., with an increasing dose level, the survival of the mice was significantly improved. Specifically, on Day 21 post grouping, the survived number of mice were: 3 in the G1 group (PBS); 2 in the G2 group (0.1 mg/kg); 5 in the G3 group (0.3 mg/kg) with an observable therapeutic effect (TGI_{TV}%=29.7%); 6 in the G4 group (1 mg/kg) with a significant treatment effect (TGI_{TV}%=62.6%); 7 in the G5 group (3 mg/kg) including 2 tumor-free mice, with a significant treatment effect (TGI_{TV}%=78.3%); 7 in the G6 group (10 mg/kg) including 5 tumor-free mice, with a significant treatment effect (TGI_{TV}%=99.4%); and 7 in the G7 group (30 mg/kg) including 4 tumor-free mice, with a significant treatment effect (TGI_{TV}%=87.3%).

The rest of the mice (mice not survived) were euthanized because the tumor size exceeded 3000 mm³ by Day 21. Because all mice in the control group reached the standard of euthanasia on Day 21 post grouping, the tumor size and TGI_{TV}% on Day 18 and Day 21; and the survival status at the end of the experimental period (on Day 70) were summarized as follows.

TABLE 21

| | | | | | | | | | | Tumor | | |
|---|---|---|---|---|---|---|---|---|---|---|---|---|
| | | Tumor volume | | | | | Last | | | free | P value on | |
| | | (mm³) | | | Survival | Survival | survival | TGI_{TV} | TGI_{TV} | on | Day 21 | |
| | | Day | Day | Day | on Day | on Day | time | % on Day | % on Day | Day | Body | Tumor |
| | | 0 | 18 | 21 | 21 | 70 | (Day) | 18 | 21 | 21 | weight | Volume |
| Control | G1 | 124 ± 4 | 2853 ± 130 | 3215 ± 97 | 3/7 | 0/7 | 21 | n.a. | n.a. | 0 | n.a. | n.a. |
| Treat | G2 | 124 ± 4 | 3605 ± 311 | 3622 ± 335 | 2/7 | 0/7 | 21 | −27% | −13% | 0 | 0.953 | 0.241 |
| | G3 | 124 ± 5 | 2094 ± 443 | 2298 ± 424 | 5/7 | 0/7 | 28 | 27.8% | 29.7% | 0 | 0.272 | 0.159 |
| | G4 | 124 ± 5 | 1337 ± 634 | 1281 ± 412 | 6/7 | 0/7 | 39 | 55.6% | 62.6% | 0 | 0.130 | 0.015 |
| | G5 | 124 ± 5 | 698 ± 328 | 796 ± 376 | 7/7 | 2/7 | 70 | 79.0% | 78.3% | 2 | 0.005 | 0.004 |
| | G6 | 124 ± 5 | 117 ± 87 | 143 ± 117 | 7/7 | 4/7 | 70 | 100.2% | 99.4% | 5 | 0.082 | 2.45E−07 |
| | G7 | 124 ± 7 | 404 ± 292 | 517 ± 361 | 7/7 | 4/7 | 70 | 89.7% | 87.3% | 4 | 0.019 | 1.20E−06 |

At the end of the experimental period, all mice in groups G1-G4 were euthanized due to excessive tumor volume; and the number of survived mice in groups G5-G7 were 2, 4, and 4, respectively. This indicates that the efficacy of the bispecific antibody ScFV-HC-IgG1-LALA in mice is correlated with the dosage amount. In addition, the therapeutic effects of ScFV-HC-IgG1-LALA in the G6 group (10 mg/kg) and the G7 group (30 mg/kg) were similar, indicating that 10 mg/kg can be a saturated dose level for ScFV-HC-IgG1-LALA in vivo.

### Example 12. Tumor-Infiltrating Lymphocytes (TILs) Analysis

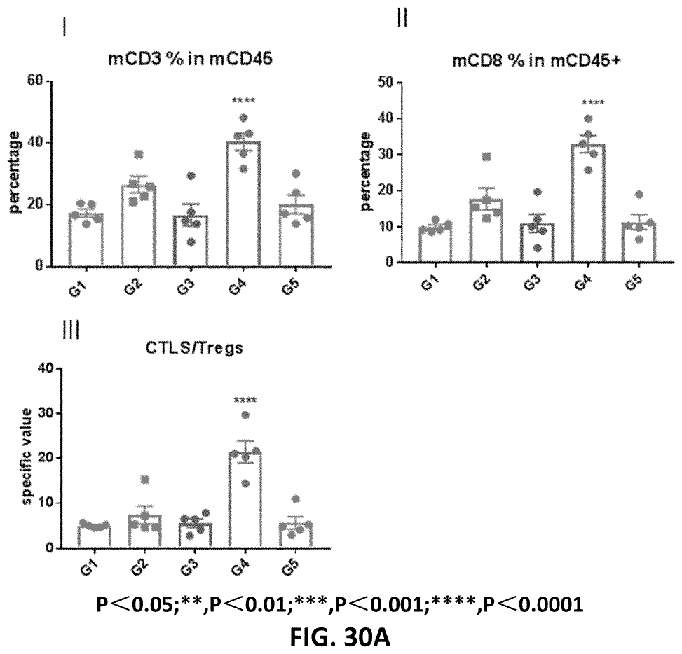

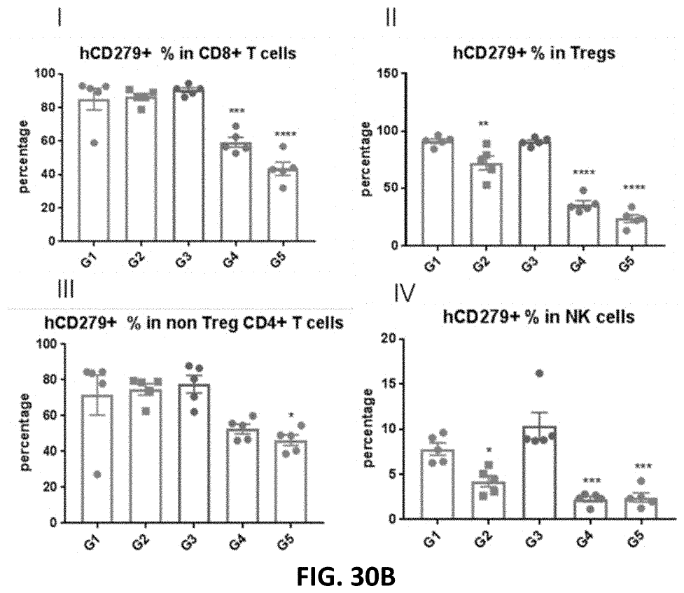

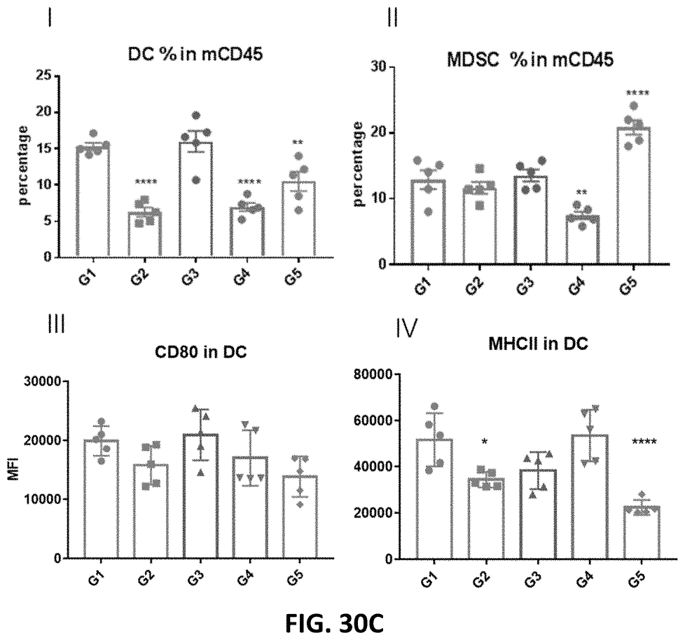

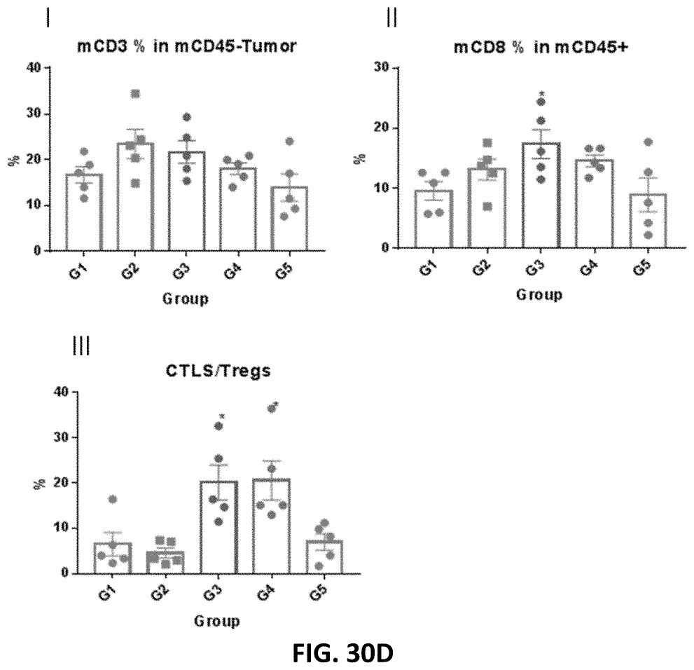

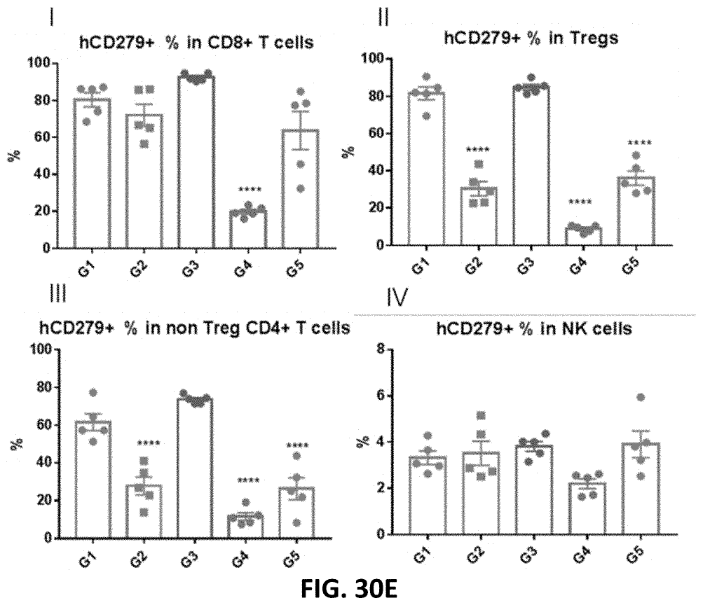

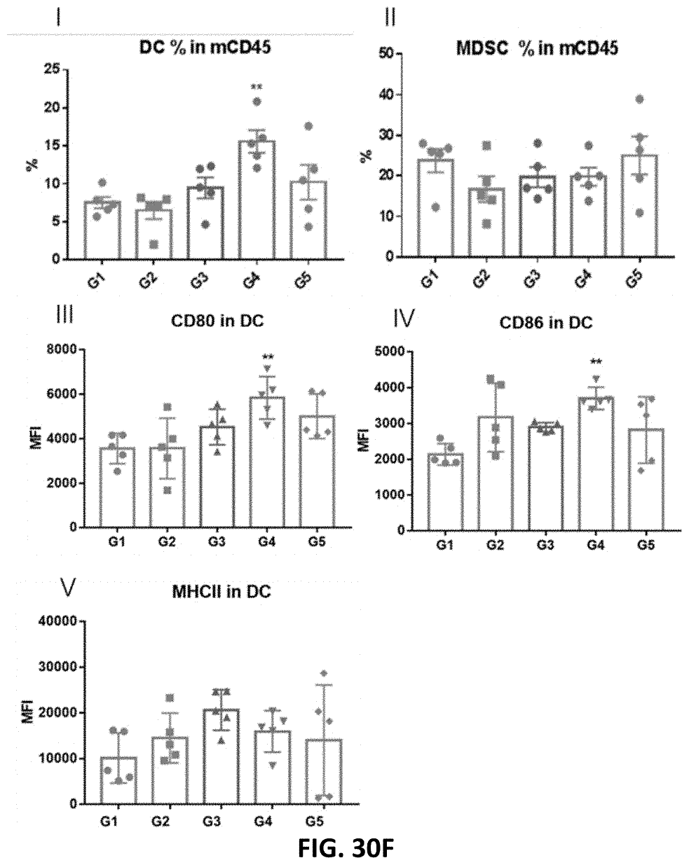

TILs analysis was performed as follows. MC38-hPD-L1 cells were injected subcutaneously in double humanized CD40/PD-1 mice (B-hPD-1/hCD40 mice). On Day 11, Day 14, and Day 18 post the injection, PBS (G1), 3 mg/kg anti-PD-1 monoclonal antibody 1A7-H2K3-IgG4 (G2), 3 mg/kg anti-CD40 monoclonal antibody 6A7-H4K2-IgG2 (G3), 4 mg/kg bispecific antibody ScFV-HC-IgG1-LALA (G4), or 3 mg/kg anti-PD-1 monoclonal antibody 1A7-H2K3-IgG4 in combination with 3 mg/kg anti-CD40 monoclonal antibody 6A7-H4K2-IgG2 (G5) was administered by intraperitoneal (i.p.) administration. On Day 21 post the injection, the tumor tissues from the control group G1 and the treatment groups G2-G5 were subjected to TILs analysis. The test results are shown in FIGS. 30A-30C.

In a similar experiment, B16F10-hPD-L1 cells were injected subcutaneously in double humanized CD40/PD-1 mice (B-hPD-1/hCD40 mice). On Day 9 and Day 13 post the injection, PBS (G1), 3 mg/kg anti-PD-1 monoclonal antibody 1A7-H2K3-IgG4 (G2), 3 mg/kg anti-CD40 monoclonal antibody 6A7-H4K2-IgG2 (G3), 4 mg/kg bispecific antibody ScFV-HC-IgG1-LALA (G4), or 3 mg/kg anti-PD-1 monoclonal antibody 1A7-H2K3-IgG4 in combination with 3 mg/kg anti-CD40 monoclonal antibody 6A7-H4K2-IgG2 (G5) was administered by intraperitoneal (i.p.) administration. On Day 15 post the injection, the tumor tissues from the control group G1 and treatment groups G2-G5 were subjected to TILs analysis. The test results are shown in FIGS. 30D-30F.

According to the results in FIG. 30A-III and FIG. 30D-III, the bispecific antibody ScFV-HC-IgG1-LALA group (G4) effectively increased the ratio of CD8+ T cells to Tregs in T cells (CTLS/Tregs) in both the MC38 tumor model and the B16F10 tumor model, as compared to that of the control group (G1).

In addition, the analysis results of PD-1 expression (FIG. 30B and FIG. 30E) showed that in the MC38 tumor model and the B16F10 tumor model, the proportion of PD-1 positive cells within CD8+ T cells, Treg cells, CD4+ (non-Treg) T cells, or NK cells in the bispecific antibody group (G4) was significantly reduced as compared to that of the monoclonal antibody group (G2 or G3) and the control group (G1). Particularly in the B16F10 tumor model, the proportion of PD-1 positive cells within CD8+ T cells was significantly reduced (P<0.0001, see FIG. 30E-I), indicating that PD-1 expression was down-regulated on the surface of these cells after treatment with ScFV-HC-IgG1-LALA.

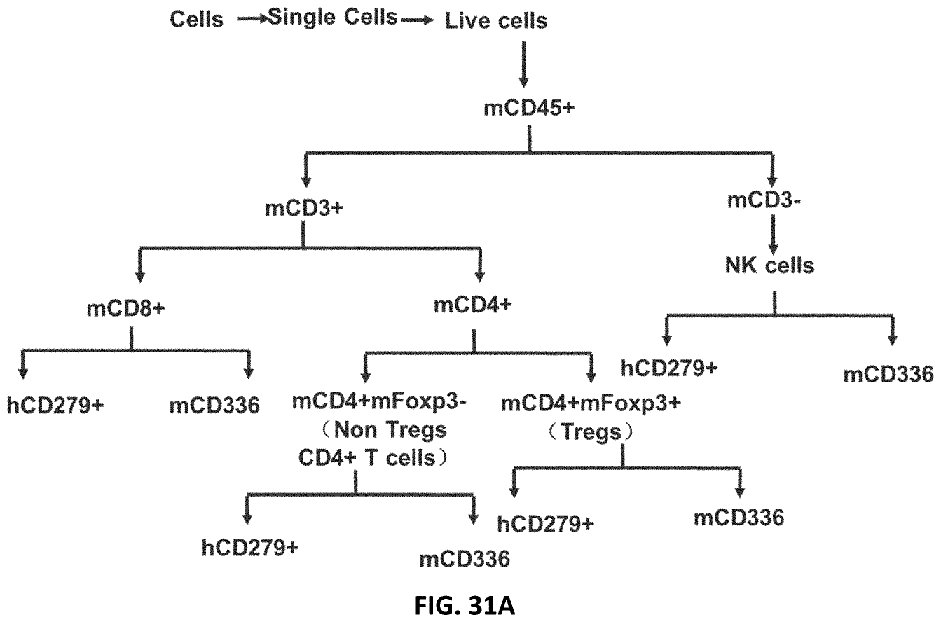

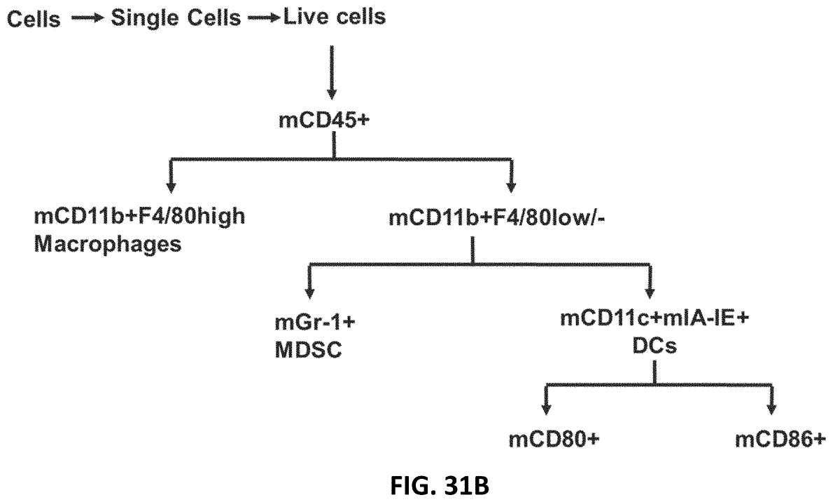

Further, the bone marrow-derived cells was analyzed. In the MC38 tumor model, the percentages of dendritic cells (DC) and myeloid-derived suppressor cells (MDSC) in leukocytes (CD45+) showed a downward trend (FIG. 30C-I and FIG. 30C-II). By contrast, in the B 16F10 tumor model, the percentages of these two cell types in leukocytes (CD45+) showed an upward trend (FIG. 30F-I and FIG. 30F-II). Combined with the detection results of DC cell activation (CD80/CD86+DC and MHCII+DC) in the MC38 tumor model (FIG. 30C-III and FIG. 30C-IV) and in the B16F10 tumor model (FIG. 30F-III and FIG. 30F-IV), it was found that in the MC38 tumor model, the bispecific antibody group (G4) exhibited a better effect to promote the proliferation/infiltration of killer T cells, and to reduce bone marrow-derived inhibitory cells (myeloid-derived suppressor cells, MDSC) ratio, as compared to that of the monoclonal antibody group (G2 or G3) and the antibody combination group (G5); in the B16F10 tumor model, the bispecific antibody group (G4) mainly promoted the proliferation/infiltration and activation of DC cells, as well as the down-regulation of the ratio of PD-1 in CD8+ T cells, as compared to that of the monoclonal antibody group (G2 or G3) and the antibody combination group (G5). The MC38 tumor model has the characteristics of a hot tumor. The results indicate that in a hot tumor model, the bispecific antibody can effectively promote the proliferation and infiltration of CD8+ T cells and reduce the number of myeloid-derived suppressor cells. In the B 16F10-PDL1 tumor model, which has the characteristics of a cold tumor, the bispecific antibody can effectively promote the proliferation, infiltration and activation of DC cells, and downregulate the expression of PD1 in CD8+ T cells. The strategy of flow cytometry analysis is shown in FIGS. 31A-31B.

### Example 13. Replacement of Different PD-1 Antibodies in the ScFV-HC-IgG Structure

Two of the marketed anti-PD-1 monoclonal antibodies, toripalimab and pembrolizumab, were selected, and their variable regions were used to replace the anti-PD-1 variable regions of the ScFV-HC-IgG1-LALA bispecific antibody according to their sequences. The resulting antibodies were named Toripalimab-6A7-HC-IgG1-LALA (with heavy chain sequence set forth in SEQ ID NO: 162 and light chain sequence set forth in SEQ ID NO: 163) and Pembrolizumab-6A7-HC-IgG1-LALA (with heavy chain sequence set forth in SEQ ID NO: 164 and light chain sequence set forth in SEQ ID NO: 165), respectively.

Similar to the previous in vivo drug efficacy experiments, the bispecific antibodies Toripalimab-6A7-HC-IgG1-LALA and Pembrolizumab-6A7-HC-IgG1-LALA were tested for their effect on tumor growth in vivo in a mouse model of colon carcinoma. MC-38 cancer tumor cells (colon adenocarcinoma cell) expressing human PD-L1 (MC38-hPD-L1) were injected subcutaneously in double humanized CD40/PD-1 mice (B-hPD-1/hCD40 mice). When the tumors in the mice reached a volume of about 400 mm³, the mice were randomly placed into different groups (6 mice per group) based on the tumor volume.

In each group, B-hPD-1/hCD40 mice were injected with phosphate-buffered saline (PBS, G1), 1 mg/kg anti-CD40 monoclonal antibody 6A7-H4K2-IgG2 (G2), 1 mg/kg anti-PD1 monoclonal antibody Toripalimab (G3), 1 mg/kg anti-PD1 monoclonal antibody Pembrolizumab (G4), 1.35 mg/kg (based on similar molar amount) Toripalimab-6A7-HC-IgG1-LALA (G5), 1.35 mg/kg Pembrolizumab-6A7-HC-IgG1-LALA(G6), combination of 1 mg/kg anti-PD1 monoclonal antibody Toripalimab and 1 mg/kg anti-CD40 monoclonal antibody 6A7-H4K2-IgG2 (G7), or combination of 1 mg/kg anti-PD1 monoclonal antibody Pembrolizumab and 1 mg/kg anti-CD40 monoclonal antibody 6A7-H4K2-IgG2 (G8) by intraperitoneal (i.p.) administration. The frequency of administration was twice a week (5 administrations in total). Details are shown in the table below.

TABLE 22

| | | | | | | Total No. |
|---|---|---|---|---|---|---|
| | | Dosage | | | Fre- | of admin- |
| Group | Antibodies | (mg/kg) | | Route | quency | istration |
| G1 | PBS | — | | i.p. | BIW | 5 |
| G2 | 6A7-H4K2-IgG2 | 1 | mg/kg | i.p. | BIW | 5 |
| G3 | Toripalimab | 1 | mg/kg | i.p. | BIW | 5 |
| | (IgG4) | | | | | |
| G4 | Pembrolizumab | 1 | mg/kg | i.p | BIW | 5 |
| | (IgG4) | | | | | |
| G5 | Toripalimab-6A7- | 1.35 | mg/kg | i.p. | BIW | 5 |
| | HC-IgG1-LALA | | | | | |
| G6 | Pembrolizumab- | 1.35 | mg/kg | i.p. | BIW | 5 |
| | 6A7-HC-IgG1- | | | | | |
| | LALA | | | | | |
| G7 | Toripalimab + | 1 + 1 | mg/kg | i.p. | BIW | 5 |
| | 6A7-H4K2-IgG2 | | | | | |
| G8 | Pembrolizumab + | 1 + 1 | mg/kg | i.p. | BIW | 5 |

6A7-H4K2-IgG2

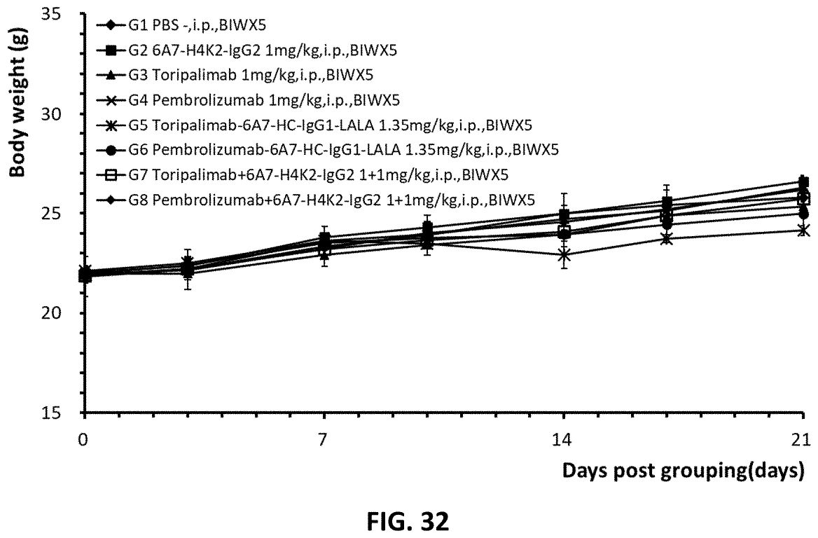

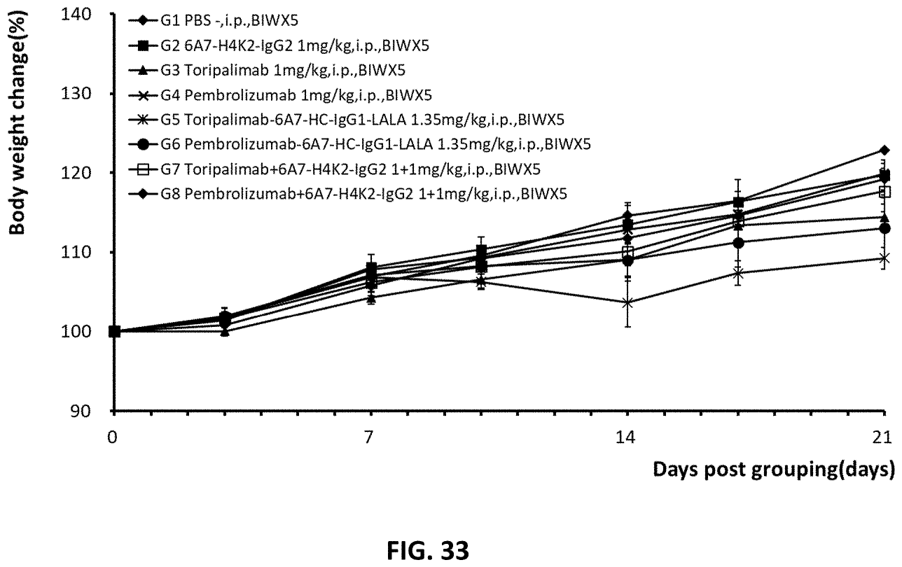

The weight of the mice was monitored during the entire treatment period. The average weight of mice in different groups all increased to different extents (FIG. 32, and FIG. 33). All the mice gained weight among different groups at the end of the treatment period.

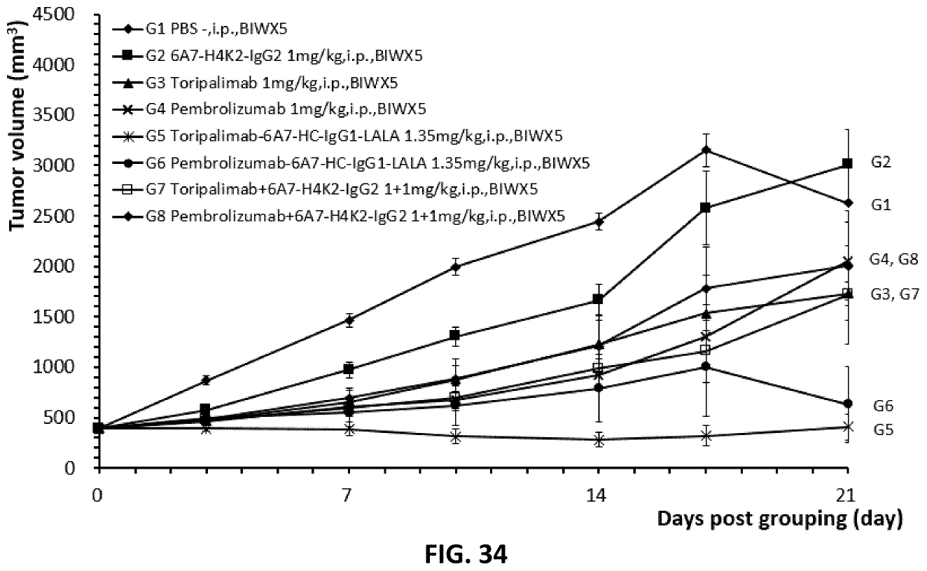

The tumor size in groups treated with the antibodies is shown in FIG. 34. The TGI_{TV}% on Day 17 and Day 21 (17 days and 21 days after grouping) was also calculated as shown in the table below. As a large number of mice in the control group were euthanized due to the tumor volume exceeding the limit, P values for Day 21 were not available. P values in the following table was calculated based on the data on Day 17.

TABLE 23

| | | Tumor volume | | | | | | | | P value for | |
|---|---|---|---|---|---|---|---|---|---|---|---|
| | | (mm³) | | | | Survival | Survival | TGI_{TV} | TGI_{TV} | Day 17 | |
| | | Day | Day | Day | Day | on Day | on Day | % on | % on | Body | Tumor |
| | | 0 | 14 | 17 | 21 | 17 | 21 | Day 17 | Day 21 | weight | Volume |
| Control | G1 | 389 ± 36 | 2443 ± 81 | 3152 ± 163 | 2628 | 6/6 | 1/6 | n.a. | n.a. | n.a. | n.a. |
| Treat | G2 | 389 ± 7 | 1662 ± 156 | 2578 ± 362 | 3006 ± 354 | 6/6 | 4/6 | 20.8% | −16.9% | 0.780 | 0.179 |
| | G3 | 389 ± 9 | 1225 ± 300 | 1534 ± 376 | 1728 ± 113 | 6/6 | 5/6 | 58.6% | 40.2% | 0.396 | 0.003 |
| | G4 | 389 ± 7 | 926 ± 154 | 1304 ± 321 | 2409 ± 386 | 6/6 | 6/6 | 66.9% | 25.9% | 0.723 | 4.46E−04 |
| | G5 | 389 ± 9 | 282 ± 77 | 320 ± 105 | 406 ± 133 | 6/6 | 6/6 | 102.5% | 99.3% | 0.011 | 4.52E−08 |
| | G6 | 389 ± 8 | 788 ± 334 | 1003 ± 495 | 630 ± 377 | 6/6 | 5/6 | 77.8% | 89.2% | 0.288 | 0.02 |
| | G7 | 389 ± 11 | 986 ± 210 | 1160 ± 309 | 1720 ± 486 | 6/6 | 6/6 | 72.1% | 40.6% | 0.502 | 1.97E−04 |
| | G8 | 389 ± 8 | 1208 ± 253 | 1778 ± 414 | 2005 ± 540 | 6/6 | 4/6 | 49.7% | 27.8% | 0.787 | 0.011 |

The results showed that Toripalimab-6A7-HC-IgG1-LALA (G5) and pembrolizumab-6A7-HC-IgG1-LALA(G6) both inhibited tumor growth with a higher TGI_{TV}% (e.g., on Day 21) than that of the monoclonal antibodies (G2, G3, and G4) and the antibody combinations (G7 and G8). In particular, all mice in the G5 group survived and the tumor in one mouse disappeared completely. The results indicates that different anti-PD-1 antigen binding fragments can be used with the anti-CD40 scFv on the structure of ScFV-HC-IgG to obtain better efficacy.

### Example 14. Replacement of Different CD40 scFv in the ScFV-HC-IgG Structure

CD40 is a key immune co-stimulatory pathway receptor, which exists on the surface of antigen-presenting cells (APC) in the immune system, and plays a key role in the activation of the innate and adaptive immune system mechanisms. Anti-CD40 antibodies that are currently under development include, e.g., APX005M (VH SEQ ID NO: 191; VL SEQ ID NO: 192) developed by Apexigen, RG7876 (selicrelumab) developed by Roche, VIB4920 developed by Viela Bio, and ADC-1013 developed by Alligator Biosciences. In this experiment, two of the anti-CD40 monoclonal antibodies, selicrelumab and APX005M were selected to generate bispecific antibodies in combination with pembrolizumab. The resulting antibodies have a structure of ScFV-HC-IgG, as shown in FIG. 1B, and the antibodies were named as Pembrolizumab-Seli-FVHC-IgG4 (with heavy chain sequence set forth in SEQ ID NO: 175 and light chain sequence set forth in SEQ ID NO: 176) and Pembrolizumab-APX005M-FVHC-IgG4 (with heavy chain sequence set forth in SEQ ID NO: 177 and light chain sequence set forth in SEQ ID NO: 178), respectively. In addition, the PD-1 antigen binding sites and the CD40 antigen binding sites in 1A7-selicrelumab-FVHC-IgG4 were exchanged, generating Selicrelumab-1A7-FVHC-IgG4 (with heavy chain sequence set forth in SEQ ID NO: 201 and light chain sequence set forth in SEQ ID NO: 202)

The bispecific antibodies Pembrolizumab-Seli-FVHC-IgG4 and Pembrolizumab-APX005M-FVHC-IgG4 were tested for their effect on tumor growth in vivo in a mouse model of colon carcinoma. Specifically, MC-38 cancer tumor cells (colon adenocarcinoma cell) expressing human PD-L1 (MC38-hPD-L1) were injected subcutaneously in double humanized CD40/PD-1 mice (B-hPD-1/hCD40 mice). When the tumors in the mice reached a volume of about 400 mm³, the mice were randomly placed into different groups (6 mice per group) based on the volume of the tumor.

Similar to the previous in vivo drug efficacy experiments, the bispecific antibodies Pembrolizumab-Seli-FVHC-IgG4, Pembrolizumab-APX005M-FVHC-IgG4 and Selicrelumab-1A7-FVHC-IgG4 were tested for their effect on tumor growth in vivo in a mouse model of colon carcinoma. MC-38 cancer tumor cells (colon adenocarcinoma cell) expressing human PD-L1 (MC38-hPD-L1) were injected subcutaneously in double humanized CD40/PD-1 mice (B-hPD-1/hCD40 mice). When the tumors in the mice reached a volume of about 400 mm³, the mice were randomly placed into different groups (6 mice per group) based on the tumor volume.

In each group, B-hPD-1/hCD40 mice were injected with physiological saline (PS, G1), 1 mg/kg anti-PD1 monoclonal antibody Pembrolizumab (G2), 1 mg/kg anti-CD40 monoclonal antibody APX005M (IgG1-S267E) (G3), selicrelumab-IgG2 (G4), 1.35 mg/kg Pembrolizumab-Seli-FVHC-IgG4 (G5), 1.35 mg/kg Pembrolizumab-APX005M-FVHC-IgG4 (G6), 1.35 mg/kg Selicrelumab-1A7-FVHC-IgG4 (G7), combination of 1 mg/kg anti-PD1 monoclonal antibody Pembrolizumab and 1 mg/kg anti-CD40 monoclonal antibody APX005M (G8), or combination of 1 mg/kg anti-PD1 monoclonal antibody Pembrolizumab and 1 mg/kg anti-CD40 monoclonal antibody selicrelumab (G8) by intraperitoneal (i.p.) administration. The frequency of administration was twice a week (6 administrations in total). Details are shown in the table below.

TABLE 23

| | | | | | | Total No. |
|---|---|---|---|---|---|---|
| | | Dosage | | | Fre- | of admin- |
| Group | Antibodies | (mg/kg) | | Route | quency | istration |
| G1 | PS | — | | i.p. | BIW | 6 |
| G2 | pembrolizumab- | 1 | mg/kg | i.p. | BIW | 6 |
| | IgG4 | | | | | |
| G3 | APX005M-IgG1- | 1 | mg/kg | i.p. | BIW | 6 |
| | S267E | | | | | |
| G4 | selicrelumab- | 1 | mg/kg | i.p. | BIW | 6 |
| | IgG2 | | | | | |
| G5 | Pembrolizumab- | 1.35 | mg/kg | i.p. | BIW | 6 |
| | Seli-FVHC- | | | | | |
| | IgG4 | | | | | |
| G6 | Pembrolizumab- | 1.35 | mg/kg | i.p. | BIW | 6 |
| | APX005M- | | | | | |
| | FVHC-IgG4 | | | | | |
| G7 | Selicrelumab- | 1.35 | mg/kg | i.p. | BIW | 6 |
| | 1A7-FVHC-IgG4 | | | | | |
| G8 | pembrolizumab- | 1 + 1 | mg/kg | i.p. | BIW | 6 |
| | IgG4 + | | | | | |
| | APX005M-IgG1- | | | | | |
| | S267E | | | | | |
| G9 | pembrolizumab- | 1 + 1 | mg/kg | i.p. | BIW | 6 |
| | IgG4 + | | | | | |

selicrelumab-
IgG2

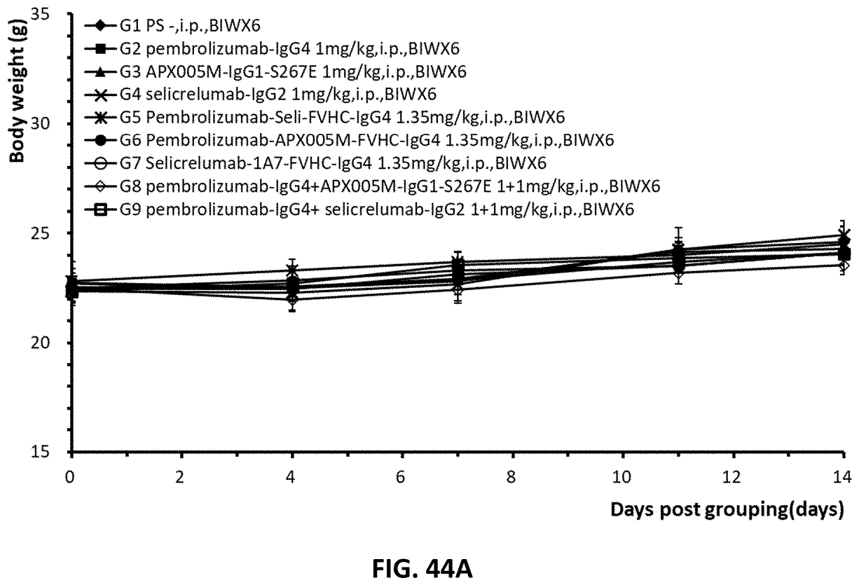

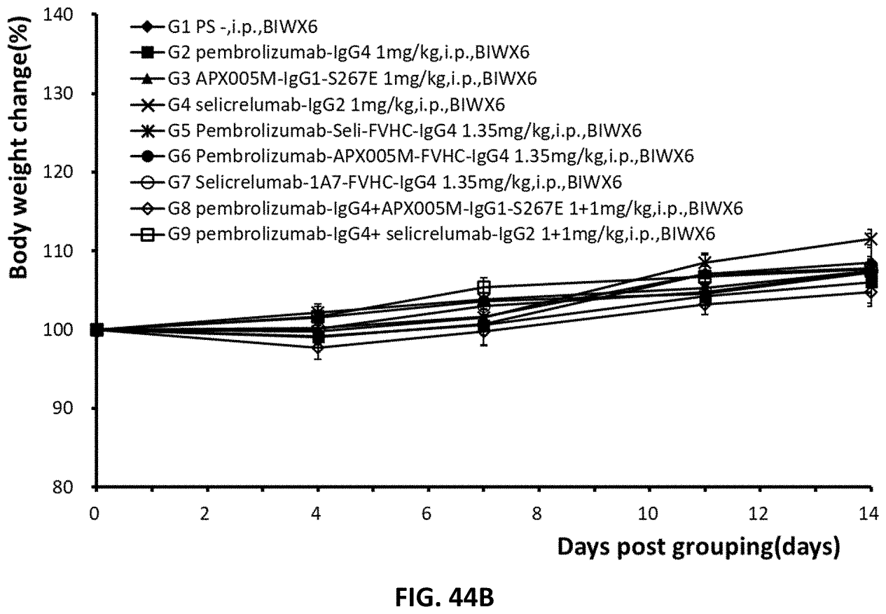

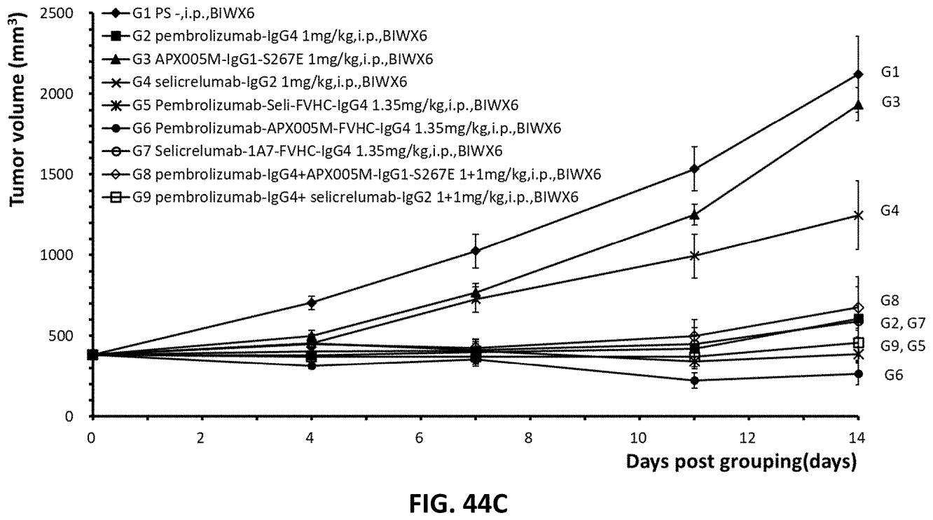

14 days after the grouping, the average weight for each group are not obviously different (FIG. 44A and FIG. 44B). All mice in different groups gained weight. The tumor size for each group is shown in FIG. 44C. The results showed that Pembrolizumab-APX005M-FVHC-IgG4 (G6) had better efficacy than monoclonal antibodies (G2) and (G3) or the combination therapy (G8).

### Example 15. In Vivo Toxicity Testing for Bispecific Antibodies with Different Structures

Multiple structural formats of PD-1/CD40 bispecific antibodies were constructed. Specifically, the sequences of scFv of Selicrelumab and the anti-PD-1 monoclonal antibody 1A7 were used to generate four bispecific antibodies with different structures and their toxicity was tested. The obtained antibodies were named as follows: 1A7-selicrelumab-FVKH-IgG4 (structure shown in FIG. 1A, with heavy chain sequence set forth in SEQ ID NO: 166, SEQ ID NO: 173 and light chain sequence set forth in SEQ ID NO: 167); 1A7-selicrelumab-FV3A-IgG4 (structure shown in FIG. 18A, with heavy chain sequence set forth in SEQ ID NO: 168 and light chain sequence set forth in SEQ ID NO: 169), which comprised a replacement of the anti-CD40 antibody 6A7 sequences with the corresponding sequences of selicrelumab; 1A7-selicrelumab-DART-IgG4 (structure shown in FIG. 37, with heavy chain sequence set forth in SEQ ID NO: 170 and light chain sequence set forth in SEQ ID NO: 171); 1A7-selicrelumab-FVHC-IgG4 (structure shown in FIG. 1B, with heavy chain sequences set forth in SEQ ID NOs: 172, and light chain sequence set forth in SEQ ID NO: 174).

It was known in the field that Selicrelumab is relatively toxic. The toxicity of multiple structural formats of PD-1/CD40 bispecific antibodies were tested in mice. Double humanized CD40/PD-1 mice (about 7-week old) were randomly placed into 8 groups (3 mice per group) according to their body weight. One of the following antibodies was randomly selected and administered on the day of grouping and every 3 days thereafter: anti-CD40 monoclonal antibody Selicrelumab (G2), anti-PD-1 monoclonal antibody 1A7-H2K3-IgG4 (G3), 1A7-selicrelumab-FVHC-IgG4 (G4), 1A7-selicrelumab-FV3A-IgG4 (G5), 1A7-selicrelumab-DART-IgG4 (G6), 1A7-selicrelumab-FVKH-IgG4 (G7), and bispecific antibody ScFV-HC-IgG1-LALA (G8). The control group (G1) was injected with PBS.

TABLE 25

| | | | | | Total No. |
|---|---|---|---|---|---|
| | | Dosage | | Fre- | of admin- |
| Group | Antibodies | (mg/kg) | Route | quency | istration |
| G1 | PBS (control) | — | i.p. | Day 0, | 3 |
| | | | | Day 3, | |
| | | | | Day 6 | |
| G2 | Selicrelumab | 10 mg/kg | i.p. | Day 0, | 3 |
| | | | | Day 3, | |
| | | | | Day 6 | |
| G3 | 1A7-H2K3-IgG4 | 10 mg/kg | i.p | Day 0, | 3 |
| | | | | Day 3, | |
| | | | | Day 6 | |
| G4 | 1A7-selicrelumab- | 13 mg/kg | i.p. | Day 0, | 3 |
| | FVHC-IgG4 | | | Day 3, | |
| | | | | Day 6 | |
| G5 | 1A7-selicrelumab- | 13 mg/kg | i.p. | Day 0, | 3 |
| | FV3A-IgG4 | | | Day 3, | |
| | | | | Day 6 | |
| G6 | 1A7-selicrelumab- | 13 mg/kg | i.p. | Day 0, | 3 |
| | DART-IgG4 | | | Day 3, | |
| | | | | Day 6 | |
| G7 | 1A7-selicrelumab- | 10 mg/kg | i.p. | Day 0, | 3 |
| | FVKH-IgG4 | | | Day 3, | |
| | | | | Day 6 | |
| G8 | ScFV-HC-IgG1-LALA | 13 mg/kg | i.p. | Day 0, | 3 |
| | | | | Day 3, | |
| | | | | Day 6 | |

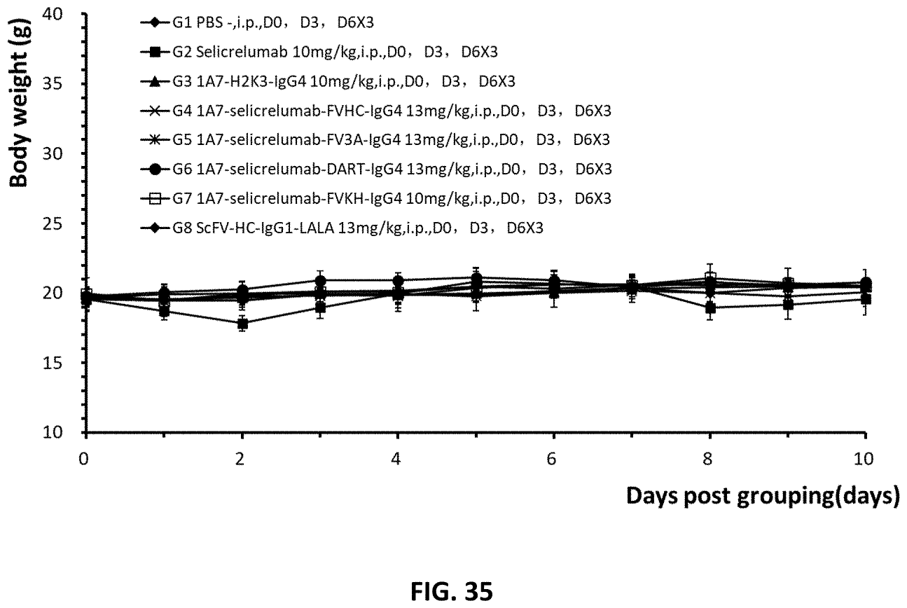

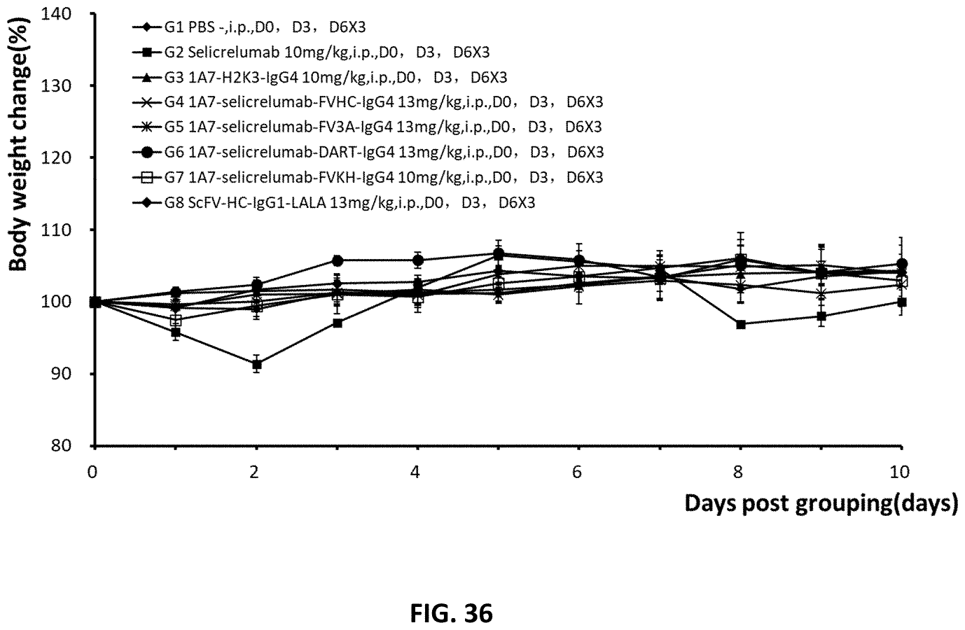

As shown in FIGS. 35-36, the experimental results showed that only the G2 group mice showed significant weight loss, and the body weight of mice in other treatment groups showed no significant difference as compared with the control group mice.

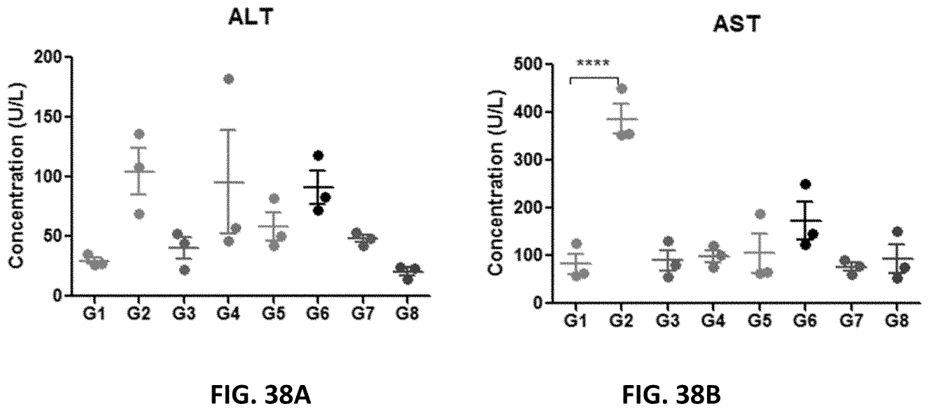

On Day 7 post grouping, peripheral blood was collected to detect the concentration of asparagine aminotransferase (AST) and alanine aminotransferase (ALT). As shown in FIGS. 38A-38B, the ALT and AST detection results showed that the G2 group mice (administered with anti-CD40 monoclonal antibody selicrelumab) had the highest concentration of both ALT and AST aminotransferases. The aminotransferase concentrations in the bispecific antibody groups (G4-G8) were lower than that in the G2 group. Specifically, only the G4 and G6 group mice showed a tendency of increasing aminotransferase concentrations. The aminotransferase concentrations in mice of the other groups were close to that of the G1 group mice.

On Day 10 post grouping, the mouse liver was isolated and examined under microscope. The results are shown in the table below, which showed that the toxicity of the bispecific antibody ScFV-HC-IgG1-LALA (G8) was lower than that of the monoclonal antibodies (G2-G3) and other bispecific antibodies (G4-G7). In fact, the bispecific antibody ScFV-HC-IgG1-LALA did not show any toxic effects.

The results indicate that PD-1/CD40 bispecific antibodies with various formats can significantly reduce toxicity of anti-CD40 antibody.)

| Group | Mouse ID | RESULT |
|---|---|---|
| G1 | 110643 | NVL* |
| | 110660 | NVL |
| | 110656 | NVL |
| G2 | 110650 | Chronic inflammation (+++) |
| | 110658 | Chronic inflammation (++) |
| | 110645 | Chronic inflammation (+++) |
| G3 | 110661 | NVL |
| | 110641 | Inflammatory cell infiltration (+) |
| | 110663 | NVL |
| G4 | 110651 | Chronic inflammation (++) |
| | 110647 | Chronic inflammation (++) |
| | 110657 | Inflammatory cell infiltration (+) |
| G5 | 110659 | Inflammatory cell infiltration (+) |
| | 110642 | NVL |
| | 110653 | Inflammatory cell infiltration (+) |
| G6 | 110649 | Chronic inflammation (++) |
| | 110644 | Inflammatory cell infiltration (+) |
| | 110648 | Chronic inflammation (++) |
| G7 | 110654 | Inflammatory cell infiltration (+) |
| | 110662 | NVL |
| | 110652 | Inflammatory cell infiltration (+) |
| G8 | 110655 | NVL |
| | 110664 | NVL |
| | 110646 | NVL |

*non-visible lesion

### Example 16. In Vivo Results for BsABs Against Colon Cancer

Similar to the previous in vivo drug efficacy experiments, the above antibodies were tested for their effect on tumor growth in vivo in a mouse model of colon carcinoma. MC-38 cancer tumor cells (colon adenocarcinoma cell) expressing human PD-L1 (MC38-hPD-L1) were injected subcutaneously in double humanized CD40/PD-1 mice (B-hPD-1/hCD40 mice). When the tumors in the mice reached a volume of about 400 mm³, the mice were randomly placed into different groups (8 mice per group) based on the tumor volume.

In each group, B-hPD-1/hCD40 mice were injected with phosphate-buffered saline (PBS, G1), 1 mg/kg anti-CD40 monoclonal antibody Selicrelumab (Selicrelumab-IgG2, G2), 1 mg/kg anti-PD-1 monoclonal antibody 1A7-H2K3-IgG4 (G3), 1 mg/kg Selicrelumab in combination with 1 mg/kg 1A7-H2K3-IgG4 (G4), 1.35 mg/kg 1A7-selicrelumab-FV3A-IgG4 (G5), or 1.35 mg/kg 1A7-selicrelumab-FVHC-IgG4 (G6) by intraperitoneal (i.p.) administration. The frequency of administration was twice a week (6 administrations in total). Details are shown in the table below.

TABLE 27

| | | | | | Total No. |
|---|---|---|---|---|---|
| | | Dosage | | Fre- | of admin- |
| Group | Antibodies | (mg/kg) | Route | quency | istration |
| G1 | PBS | — | i.p. | BIW | 6 |
| G2 | Selicrelumab | 1 mg/kg | i.p. | BIW | 6 |
| G3 | 1A7-H2K3-IgG4 | 1 mg/kg | i.p. | BIW | 6 |
| G4 | Selicrelumab + | 1 + 1 | i.p. | BIW | 6 |
| | 1A7-H2K3-IgG4 | mg/kg | | | |
| G5 | 1A7-selicrelumab- | 1.35 | i.p. | BIW | 6 |
| | FV3A-IgG4 | mg/kg | | | |
| G6 | 1A7-selicrelumab- | 1.35 | i.p. | BIW | 6 |
| | FVHC-IgG4 | mg/kg | | | |

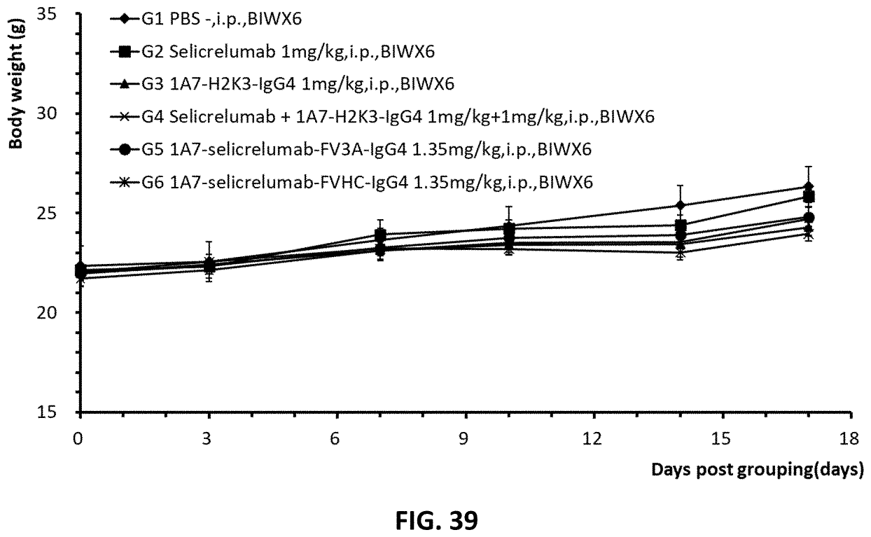

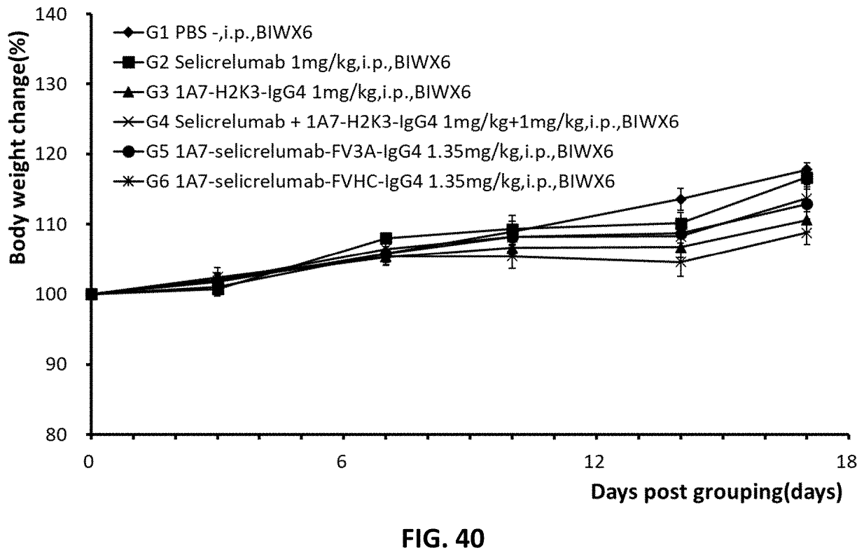

The weight of the mice was monitored during the entire treatment period. The average weight of mice in different groups all increased to different extents (FIG. 39, and FIG. 40). All the mice gained weight among different groups at the end of the treatment period.

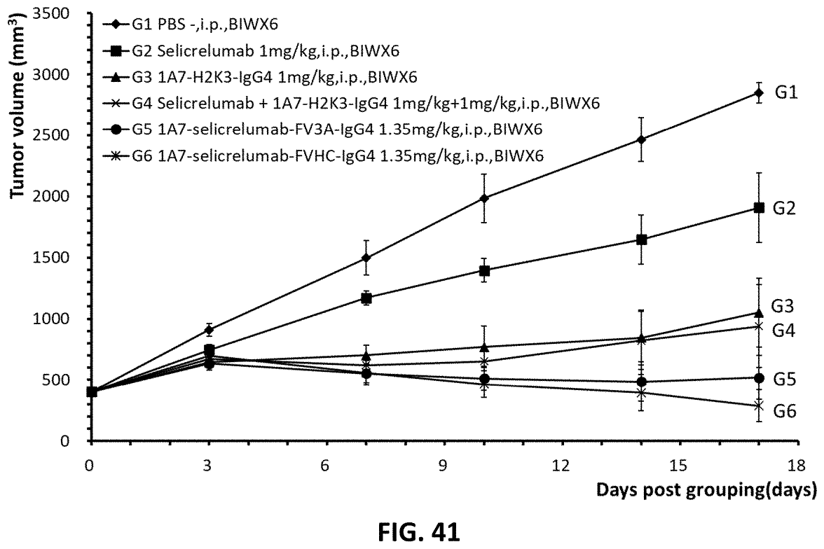

The tumor size in groups treated with the antibodies is shown in FIG. 41. The TGITV% on Day 17 (17 days after grouping) was calculated as shown in the table below. P values in the following table was calculated based on the data on Day 17

TABLE 28

| | | | | | | | P value for | |
|---|---|---|---|---|---|---|---|---|
| | | Tumor volume (mm³) | | | Survival | TGI_{TV} | Day 17 | |
| | | Day | Day | Day | on Day | % on Day | Body | Tumor |
| | | 0 | 14 | 17 | 17 | 17 | weight | Volume |
| Control | G1 | 402 ± 14 | 2464 ± 444 | 2849 ± 183 | 5/6 | n.a. | n.a. | n.a. |
| Treat | G2 | 402 ± 14 | 1647 ± 495 | 1907 ± 701 | 6/6 | 38.5% | 0.573 | 0.018 |
| | G3 | 402 ± 18 | 845 ± 545 | 1051 ± 689 | 6/6 | 73.5% | 0.007 | 3.25E−04 |
| | G4 | 402 ± 19 | 822 ± 578 | 939 ± 826 | 6/6 | 78.1% | 0.106 | 0.001 |
| | G5 | 402 ± 25 | 485 ± 396 | 519 ± 438 | 6/6 | 95.2% | 0.088 | 1.56E−06 |
| | G6 | 402 ± 18 | 394 ± 363 | 288 ± 320 | 6/6 | 104.7% | 0.002 | 7.22E−08 |

The results showed that 1A7-selicrelumab-FV3A-IgG4 (G5) and 1A7-selicrelumab-FVHC-IgG4 (G6) both inhibited tumor growth with a higher TGI_{TV}% (e.g., on Day 17) than that of the monoclonal antibodies (G2 and G3) and the antibody combinations (G4). The results indicate that different structural formats of PD-1/CD40 bispecific antibodies can significantly inhibit tumor growth with superior efficacy.

### Example 17. T Cell/APC Cell Bridging Effect of Bispecific Antibodies

The experiment was performed to test whether CD40 activation induced by PD1/CD40 BsAbs depends on the presence of the PD-1 expressing cells. Two PD-1/CD40 bispecific antibodies were generated as follows:

Pembrolizumab-APX005M-FVHC-IgG4: The scFv sequence of APX005M was linked to the C-terminus of anti-PD-1 monoclonal antibody pembrolizumab heavy chain to obtain the bispecific antibody Pembrolizumab-APX005M-FVHC-IgG4.

SdAb-6A7-FVHC-IgG1-LALA: SdAb is an anti-PD-1 nanobody (or camelid single-domain antibody) (with single variable domain (VHH) sequence set forth in SEQ ID NO: 204 from public information), was tested. The scFv sequence of the anti-CD40 monoclonal antibody 6A7-H4K2-IgG2 was linked to the C-terminus of SdAb to obtain the bispecific antibody SdAb-6A7-FVHC-IgG1-LALA.

Effector cells Jurkat-Luc-hCD40 and basal cells Jurkat-hPD1 were seeded in a 96-well plate (cell density 5×10⁴ cells/well) and incubated at 37° C. The BsAbs Pembrolizumab-6A7-HC-IgG1-LALA, Pembrolizumab-Seli-FVHC-IgG4, SdAb-6A7-FVHC-IgG1-LALA, 1A7-selicrelumab-FV3A-IgG4, 1A7-selicrelumab-FVHC-IgG4, 1A7-selicrelumab-FVKH-IgG4, 1A7-selicrelumab-DART-IgG4, Pembrolizumab-APX005M-FVHC-IgG4; and anti-CD40 antibodies APX005M(IgG1-S267E), Selicrelumab-IgG2 and 6A7-H4K2-IgG2 were serially diluted (3-fold) with the highest concentration of 3 μg/ml. 25 μl antibody was added to each well and incubated in a 37° C. incubator for 6 hours. After the incubation, the plate was taken out, and 75 μl of Bio-lite Luciferase Assay Reagent (Vazyme Biotech Co., Ltd., Cat#: DD1201-02-AB) was added to incubate at room temperature for 5-10 minutes. The plate was then placed in a luminescence detector to detect the fluorescence signal. The control experiment was performed with similar procedures, but in the absence of basal cells Jurkat-luc-hPD1.

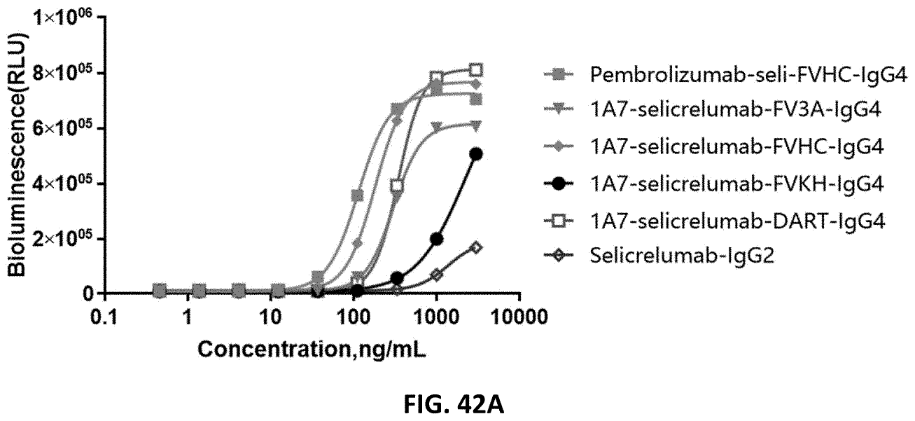

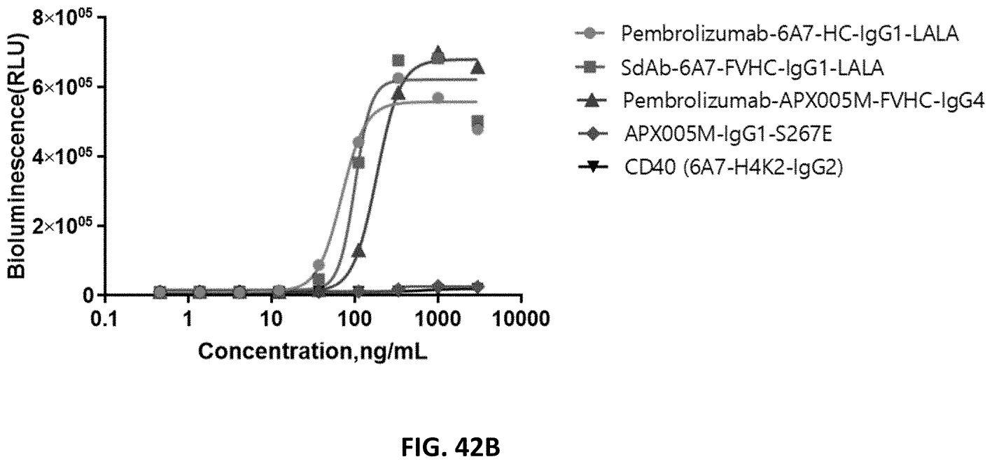

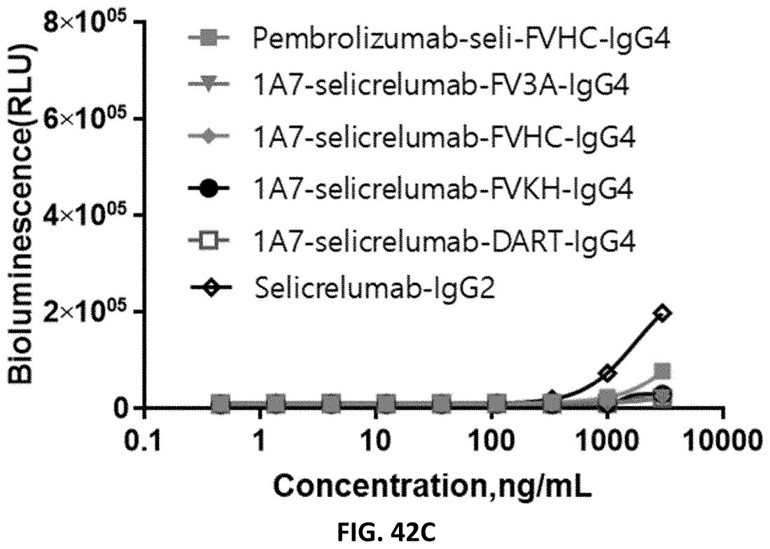

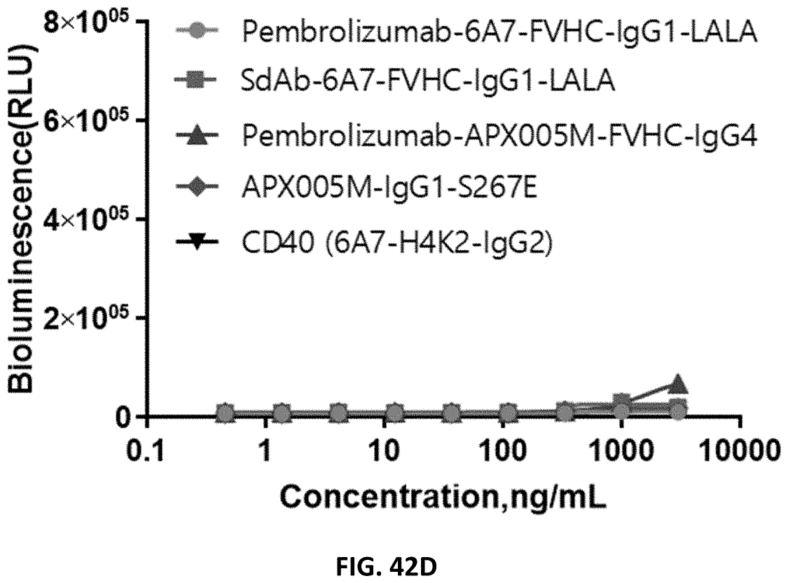

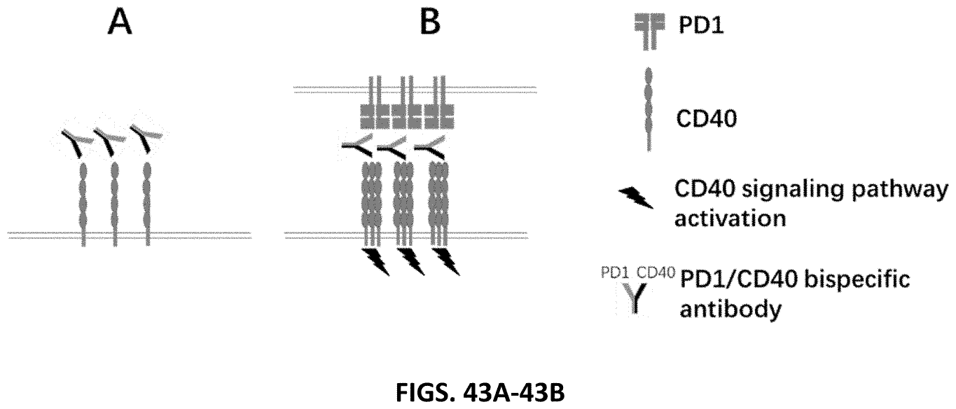

As shown in FIGS. 42A-42B, when basal cells Jurkat-luc-hPD1 were present, only BsAbs (i.e., Pembrolizumab-6A7-HC-IgG1-LALA, Pembrolizumab-Seli-FVHC-IgG4, SdAb-6A7-FVHC-IgG1-LALA, 1A7-selicrelumab-FV3A-IgG4, 1A7-selicrelumab-FVHC-IgG4, 1A7-selicrelumab-FVKH-IgG4, 1A7-selicrelumab-DART-IgG4 and Pembrolizumab-APX005M-FVHC-IgG4) activated the reporter cells in trans. By contrast, the monoclonal anti-CD40 antibodies did not activate the reporter cells except Selicrelumab-IgG2. As shown in FIGS. 42C-42D, in the absence of basal cells Jurkat-luc-hPD1, neither the BsAbs nor the monoclonal antibodies (except Selicrelumab-IgG2) activated the reporter cells.

The results indicates that activation of the CD40 pathway by the bispecific antibodies described herein depends on clustering of CD40, possibly via crosslinking with PD-1 on PD-1 expressing cells. With respect to Selicrelumab-IgG2, this anti-CD40 antibody can activate CD40 without crosslinking activity, e.g., crosslinking with FCγRIIB.

### Example 18. Replacement of Variable Domains of Various anti-PD-1 Antibodies and Anti-CD40 Antibodies

Experiments are further performed to test whether VH and VL or alternatively VHH in various anti-PD-1 antibodies can be used to replace the anti-PD-1 variable regions in the ScFV-HC-IgG1 bispecific antibody. In addition, VH and VL of various anti-CD40 monoclonal antibodies are also tested. These antibodies and their sequences are listed below.

TABLE 29

| | |
|---|---|
| Anti-PD-1 | Anti-CD40 |

| | VH | VL | VH | VL |
|---|---|---|---|---|
| | SEQ ID | SEQ ID | SEQ ID | SEQ ID |
| Structure | NO: | NO: | NO: | NO: |
| ScFV-HC-IgG1 | 179 | 180 | 191 | 192 |
| ScFV-HC-IgG1 | 181 | 182 | 191 | 192 |
| ScFV-HC-IgG1 | 183 | 184 | 191 | 192 |
| ScFV-HC-IgG1 | 185 | 186 | 191 | 192 |
| ScFV-HC-IgG1 | 187 | 188 | 191 | 192 |
| ScFV-HC-IgG1 | 189 | 190 | 191 | 192 |
| ScFV-HC-IgG1 | 207 | 208 | 191 | 192 |
| ScFV-HC-IgG1 | 209 | 210 | 191 | 192 |
| ScFV-HC-IgG1 | 207 | 208 | 191 | 192 |
| ScFV-HC-IgG1 | 209 | 210 | 191 | 192 |
| ScFV-HC-IgG1 | 179 | 180 | 193 | 194 |
| ScFV-HC-IgG1 | 181 | 182 | 193 | 194 |
| ScFV-HC-IgG1 | 183 | 184 | 193 | 194 |
| ScFV-HC-IgG1 | 185 | 186 | 193 | 194 |
| ScFV-HC-IgG1 | 187 | 188 | 193 | 194 |
| ScFV-HC-IgG1 | 189 | 190 | 193 | 194 |
| ScFV-HC-IgG1 | 207 | 208 | 193 | 194 |
| ScFV-HC-IgG1 | 209 | 210 | 193 | 194 |
| ScFV-HC-IgG1 | 207 | 208 | 193 | 194 |
| ScFV-HC-IgG1 | 209 | 210 | 193 | 194 |
| ScFV-HC-IgG1 | 179 | 180 | 195 | 196 |
| ScFV-HC-IgG1 | 181 | 182 | 195 | 196 |
| ScFV-HC-IgG1 | 183 | 184 | 195 | 196 |
| ScFV-HC-IgG1 | 185 | 186 | 195 | 196 |
| ScFV-HC-IgG1 | 187 | 188 | 195 | 196 |
| ScFV-HC-IgG1 | 189 | 190 | 195 | 196 |
| ScFV-HC-IgG1 | 207 | 208 | 195 | 196 |
| ScFV-HC-IgG1 | 209 | 210 | 195 | 196 |
| ScFV-HC-IgG1 | 207 | 208 | 195 | 196 |
| ScFV-HC-IgG1 | 209 | 210 | 195 | 196 |
| ScFV-HC-IgG1 | 179 | 180 | 197 | 198 |
| ScFV-HC-IgG1 | 181 | 182 | 197 | 198 |
| ScFV-HC-IgG1 | 183 | 184 | 197 | 198 |
| ScFV-HC-IgG1 | 185 | 186 | 197 | 198 |
| ScFV-HC-IgG1 | 187 | 188 | 197 | 198 |
| ScFV-HC-IgG1 | 189 | 190 | 197 | 198 |
| ScFV-HC-IgG1 | 207 | 208 | 197 | 198 |
| ScFV-HC-IgG1 | 209 | 210 | 197 | 198 |
| ScFV-HC-IgG1 | 207 | 208 | 197 | 198 |
| ScFV-HC-IgG1 | 209 | 210 | 197 | 198 |
| ScFV-HC-IgG1 | 179 | 180 | 199 | 200 |
| ScFV-HC-IgG1 | 181 | 182 | 199 | 200 |
| ScFV-HC-IgG1 | 183 | 184 | 199 | 200 |
| ScFV-HC-IgG1 | 185 | 186 | 199 | 200 |
| ScFV-HC-IgG1 | 187 | 188 | 199 | 200 |
| ScFV-HC-IgG1 | 189 | 190 | 199 | 200 |
| ScFV-HC-IgG1 | 207 | 208 | 199 | 200 |
| ScFV-HC-IgG1 | 209 | 210 | 199 | 200 |
| ScFV-HC-IgG1 | 204 (VHH) | n.a. | 191 | 192 |
| ScFV-HC-IgG1 | 204 (VHH) | n.a. | 193 | 194 |
| ScFV-HC-IgG1 | 204 (VHH) | n.a. | 195 | 196 |
| ScFV-HC-IgG1 | 204 (VHH) | n.a. | 197 | 198 |
| ScFV-HC-IgG1 | 204 (VHH) | n.a. | 199 | 200 |

Similar to the previous in vivo drug efficacy experiments, these bispecific antibodies are tested for their effects on tumor growth in vivo in a mouse model. Cancer cells expressing human PD-L1 (e.g., MC38-hPD-L1) are injected subcutaneously in double humanized CD40/PD-1 mice (B-hPD-1/hCD40 mice). When the tumors in the mice reach a volume of about 300-500 mm³, the mice are randomly placed into different groups (e.g., 6 mice per group) based on the tumor volume.

In each group, B-hPD-1/hCD40 mice are injected with phosphate-buffered saline (PBS, G1), 1 mg/kg anti-CD40 monoclonal antibody (G2), 1 mg/kg anti-PD1 monoclonal antibody (G3), 1.35 mg/kg ScFV-HC-IgG1-LALA (G4), and a combination of 1 mg/kg anti-PD1 monoclonal antibody and 1 mg/kg anti-CD40 monoclonal antibody (G5) by intraperitoneal (i.p.) administration. The frequency of administration is twice a week with an appropriate number of administrations (e.g., 5 administrations in total). The weight and the tumor size of each mouse are monitored during the entire treatment period. It is expected that these anti-PD1/CD40 bispecific antibodies with ScFV-HC-IgG1-LALA format (e.g., FIG. 1B) have superior efficacy in treating cancer and a low level of toxicity.

Furthermore, experiments are performed to test the efficacy of these antibodies in FV3A format (e.g., FIG. 18A). Under this structure, the anti-CD40 antigen-binding site is inserted to the 3A site of an anti-PD-1 antibody.

TABLE 30

| | Anti-PD-1 | | Anti-CD40 | |
|---|---|---|---|---|
| | VH | VL | VH | VL |
| | SEQ ID | SEQ ID | SEQ ID | SEQ ID |
| Structure | NO: | NO: | NO: | NO: |
| PD1-C40-FV3A | 179 | 180 | 191 | 192 |
| PD1-C40-FV3A | 181 | 182 | 191 | 192 |
| PD1-C40-FV3A | 183 | 184 | 191 | 192 |
| PD1-C40-FV3A | 185 | 186 | 191 | 192 |
| PD1-C40-FV3A | 187 | 188 | 191 | 192 |
| PD1-C40-FV3A | 189 | 190 | 191 | 192 |
| PD1-C40-FV3A | 207 | 208 | 191 | 192 |
| PD1-C40-FV3A | 209 | 210 | 191 | 192 |
| PD1-C40-FV3A | 179 | 180 | 193 | 194 |
| PD1-C40-FV3A | 181 | 182 | 193 | 194 |
| PD1-C40-FV3A | 183 | 184 | 193 | 194 |
| PD1-C40-FV3A | 185 | 186 | 193 | 194 |
| PD1-C40-FV3A | 187 | 188 | 193 | 194 |
| PD1-C40-FV3A | 189 | 190 | 193 | 194 |
| PD1-C40-FV3A | 207 | 208 | 193 | 194 |
| PD1-C40-FV3A | 209 | 210 | 193 | 194 |
| PD1-C40-FV3A | 179 | 180 | 195 | 196 |
| PD1-C40-FV3A | 181 | 182 | 195 | 196 |
| PD1-C40-FV3A | 183 | 184 | 195 | 196 |
| PD1-C40-FV3A | 185 | 186 | 195 | 196 |
| PD1-C40-FV3A | 187 | 188 | 195 | 196 |
| PD1-C40-FV3A | 189 | 190 | 195 | 196 |
| PD1-C40-FV3A | 207 | 208 | 195 | 196 |
| PD1-C40-FV3A | 209 | 210 | 195 | 196 |
| PD1-C40-FV3A | 179 | 180 | 197 | 198 |
| PD1-C40-FV3A | 181 | 182 | 197 | 198 |
| PD1-C40-FV3A | 183 | 184 | 197 | 198 |
| PD1-C40-FV3A | 185 | 186 | 197 | 198 |
| PD1-C40-FV3A | 187 | 188 | 197 | 198 |
| PD1-C40-FV3A | 189 | 190 | 197 | 198 |
| PD1-C40-FV3A | 207 | 208 | 197 | 198 |
| PD1-C40-FV3A | 209 | 210 | 197 | 198 |
| PD1-C40-FV3A | 179 | 180 | 199 | 200 |
| PD1-C40-FV3A | 181 | 182 | 199 | 200 |
| PD1-C40-FV3A | 183 | 184 | 199 | 200 |
| PD1-C40-FV3A | 185 | 186 | 199 | 200 |
| PD1-C40-FV3A | 187 | 188 | 199 | 200 |
| PD1-C40-FV3A | 189 | 190 | 199 | 200 |
| PD1-C40-FV3A | 207 | 208 | 199 | 200 |
| PD1-C40-FV3A | 209 | 210 | 199 | 200 |
| PD1-C40-FV3A | 204 (VHH) | n.a. | 191 | 192 |
| PD1-C40-FV3A | 204 (VHH) | n.a. | 193 | 194 |
| PD1-C40-FV3A | 204 (VHH) | n.a. | 195 | 196 |
| PD1-C40-FV3A | 204 (VHH) | n.a. | 197 | 198 |
| PD1-C40-FV3A | 204 (VHH) | n.a. | 199 | 200 |

These bispecific antibodies are tested for their effects on tumor growth in vivo in a mouse model. Cancer cells expressing human PD-L1 (e.g., MC38-hPD-L1) are injected subcutaneously in double humanized CD40/PD-1 mice (B-hPD-1/hCD40 mice). When the tumors in the mice reach a volume of about 300-500 mm³, the mice are randomly placed into different groups (e.g., 6 mice per group) based on the tumor volume.

In each group, B-hPD-1/hCD40 mice are injected with phosphate-buffered saline (PBS, G1), 1 mg/kg anti-CD40 monoclonal antibody (G2), 1 mg/kg anti-PD1 monoclonal antibody (G3), 1.35 mg/kg PD1-C40-FV3A (G4), and a combination of 1 mg/kg anti-PD1 monoclonal antibody and 1 mg/kg anti-CD40 monoclonal antibody (G5) by intraperitoneal (i.p.) administration. The frequency of administration is twice a week with an appropriate number of administrations (e.g., 5 administrations in total). The weight and the tumor size are monitored during the entire treatment period. It is expected that these anti-PD1/CD40 bispecific antibodies with PD1-C40-FV3A format (e.g., FIG. 18A) have superior efficacy in treating cancer and a low level of toxicity.

Furthermore, experiments are performed to test the efficacy of these antibodies in Fab-ScFV-IgG4 format (e.g., FIG. 1A). Under this structure, an anti-PD-1 arm comprising a heavy chain and a light chain, and an anti-CD40 arm comprising a single-chain variable fragment (scFv) connected to CH2 and CH3 domains of IgG4. Alternatively, the anti-PD-1 arm only has one heavy chain, and a VHH is connected to an optional CH1, CH2, and CH3.

TABLE 31

| | Anti-PD-1 | | Anti-CD40 | |
|---|---|---|---|---|
| | VH | VL | VH | VL |
| | SEQ ID | SEQ ID | SEQ ID | SEQ ID |
| Structure | NO: | NO: | NO: | NO: |
| Fab-ScFV-IgG4 | 179 | 180 | 191 | 192 |
| Fab-ScFV-IgG4 | 181 | 182 | 191 | 192 |
| Fab-ScFV-IgG4 | 183 | 184 | 191 | 192 |
| Fab-ScFV-IgG4 | 185 | 186 | 191 | 192 |
| Fab-ScFV-IgG4 | 187 | 188 | 191 | 192 |
| Fab-ScFV-IgG4 | 189 | 190 | 191 | 192 |
| Fab-ScFV-IgG4 | 207 | 208 | 191 | 192 |
| Fab-ScFV-IgG4 | 209 | 210 | 191 | 192 |
| Fab-ScFV-IgG4 | 179 | 180 | 193 | 194 |
| Fab-ScFV-IgG4 | 181 | 182 | 193 | 194 |
| Fab-ScFV-IgG4 | 183 | 184 | 193 | 194 |
| Fab-ScFV-IgG4 | 185 | 186 | 193 | 194 |
| Fab-ScFV-IgG4 | 187 | 188 | 193 | 194 |
| Fab-ScFV-IgG4 | 189 | 190 | 193 | 194 |
| Fab-ScFV-IgG4 | 207 | 208 | 193 | 194 |
| Fab-ScFV-IgG4 | 209 | 210 | 193 | 194 |
| Fab-ScFV-IgG4 | 179 | 180 | 195 | 196 |
| Fab-ScFV-IgG4 | 181 | 182 | 195 | 196 |
| Fab-ScFV-IgG4 | 183 | 184 | 195 | 196 |
| Fab-ScFV-IgG4 | 185 | 186 | 195 | 196 |
| Fab-ScFV-IgG4 | 187 | 188 | 195 | 196 |
| Fab-ScFV-IgG4 | 189 | 190 | 195 | 196 |
| Fab-ScFV-IgG4 | 207 | 208 | 195 | 196 |
| Fab-ScFV-IgG4 | 209 | 210 | 195 | 196 |
| Fab-ScFV-IgG4 | 179 | 180 | 197 | 198 |
| Fab-ScFV-IgG4 | 181 | 182 | 197 | 198 |
| Fab-ScFV-IgG4 | 183 | 184 | 197 | 198 |
| Fab-ScFV-IgG4 | 185 | 186 | 197 | 198 |
| Fab-ScFV-IgG4 | 187 | 188 | 197 | 198 |
| Fab-ScFV-IgG4 | 189 | 190 | 197 | 198 |
| Fab-ScFV-IgG4 | 207 | 208 | 197 | 198 |
| Fab-ScFV-IgG4 | 209 | 210 | 197 | 198 |
| Fab-ScFV-IgG4 | 179 | 180 | 199 | 200 |
| Fab-ScFV-IgG4 | 181 | 182 | 199 | 200 |
| Fab-ScFV-IgG4 | 183 | 184 | 199 | 200 |
| Fab-ScFV-IgG4 | 185 | 186 | 199 | 200 |
| Fab-ScFV-IgG4 | 187 | 188 | 199 | 200 |
| Fab-ScFV-IgG4 | 189 | 190 | 199 | 200 |
| Fab-ScFV-IgG4 | 207 | 208 | 199 | 200 |
| Fab-ScFV-IgG4 | 209 | 210 | 199 | 200 |
| Fab-ScFV-IgG4 | 204 (VHH) | n.a. | 191 | 192 |
| Fab-ScFV-IgG4 | 204 (VHH) | n.a. | 193 | 194 |
| Fab-ScFV-IgG4 | 204 (VHH) | n.a. | 195 | 196 |
| Fab-ScFV-IgG4 | 204 (VHH) | n.a. | 197 | 198 |
| Fab-ScFV-IgG4 | 204 (VHH) | n.a. | 199 | 200 |

Furthermore, experiments are performed to test the efficacy of these antibodies in Fc-containing DART format (FIG. 37) for the VH, VL, and VHH that are listed in Table 31. It is expected that these anti-PD1/CD40 bispecific antibodies also have superior efficacy in treating cancer and a low level of toxicity.

### Example 19. In Vivo Results for Bispecific Against Colon Cancer

Atezolizumab is a humanized anti-PD-L1 monoclonal antibody developed by Genentech (VH SEQ ID NO: 211; VL SEQ ID NO: 212). Avelumab is a human anti-PD-L1 monoclonal antibody developed by Merck/Pfizer (VH SEQ ID NO: 213; VL SEQ ID NO: 214). In this experiment, the VH and VL sequences of the anti-PD-1 arm of the bispecific antibodies described herein (e.g., as shown in FIG. 1B) were replaced with the VH and VL sequences of an anti-PD-Ll antibody. In addition, efficacy of the obtained PD-L1/CD40 bispecific antibody was compared to that of the corresponding PD-1/CD40 bispecific antibodies disclosed herein. Similar to the previous in vivo drug efficacy experiments, the antibodies ScFV-HC-IgG1-LALA, Atezolizumab-6A7-FVHC-IgG1-LALA (with heavy chain sequence set forth in SEQ ID NO: 215 and light chain sequence set forth in SEQ ID NO: 216) and Avelumab-6A7-FVHC-IgG1-LALA (with heavy chain sequence set forth in SEQ ID NO: 217 and light chain sequence set forth in SEQ ID NO: 218) were tested for their effect on tumor growth in vivo in a mouse model of colon carcinoma. MC-38 cancer tumor cells (colon adenocarcinoma cell) expressing human PD-L1 (MC38-hPD-L1) were injected subcutaneously in humanized PD1/PD-L1/CD40 mice (B-hPD-1/hPD-L1/hCD40 mice). When the tumors in the mice reached a volume of about 100-150 mm³, the mice were randomly placed into different groups (6 mice per group) based on the tumor volume. Details of humanized PD-L1 mouse can be found, e.g., in PCT Application No. PCT/CN2017/099574 and U.S. Pat. No. 10,945,418B2, which are incorporated herein by reference in the entirety.

In each group, B-hPD-1/hPD-L1/hCD40 mice (mice with humanized PD-1, humanized PD-L1, and humanized CD40 gene) were injected with phosphate-buffered saline (PBS, G1), 1 mg/kg anti-PD-1/CD40 antibody ScFV-HC-IgG1-LALA (G2), 1 mg/kg anti-PD-L1/CD40 bispecific antibodies Atezolizumab-6A7-FVHC-IgG1-LALA (G3), 1 mg/kg anti-PD-L1/CD40 bispecific antibodies Avelumab-6A7-FVHC-IgG1-LALA (G4), 3 mg/kg ScFV-HC-IgG1-LALA (G5), 3 mg/kg Atezolizumab-6A7-FVHC-IgG1-LALA (G6), or 3 mg/kg Avelumab-6A7-FVHC-IgG1-LALA (G7) by intraperitoneal (i.p.) administration. The frequency of administration was twice a week (4 administrations in total). Details are shown in the table below.

TABLE 32

| | | | | | Total No. |
|---|---|---|---|---|---|
| | | Dosage | | Fre- | of admin- |
| Group | Antibodies | (mg/kg) | Route | quency | istration |
| G1 | PBS | — | i.p. | BIW | 4 |
| G2 | ScFV-HC-IgG1-LALA | 1 mg/kg | i.p. | BIW | 4 |
| G3 | Atezolizumab-6A7- | 1 mg/kg | i.p. | BIW | 4 |
| | FVHC-IgG1-LALA | | | | |
| G4 | Avelumab-6A7-FVHC- | 1 mg/kg | i.p. | BIW | 4 |
| | IgG1-LALA | | | | |
| G5 | ScFV-HC-IgG1-LALA | 3 mg/kg | i.p. | BIW | 4 |
| G6 | Atezolizumab-6A7- | 3 mg/kg | i.p. | BIW | 4 |
| | FVHC-IgG1-LALA | | | | |
| G7 | Avelumab-6A7-FVHC- | 3 mg/kg | i.p. | BIW | 4 |
| | IgG1-LALA | | | | |

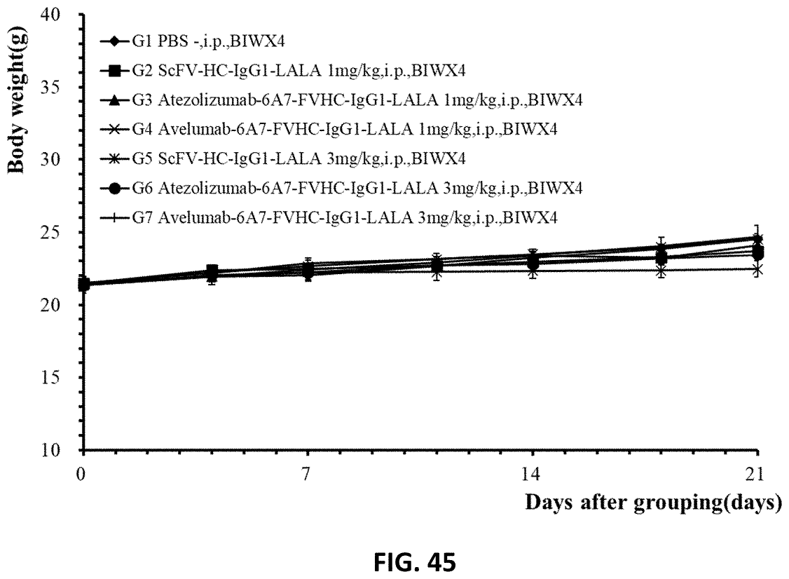

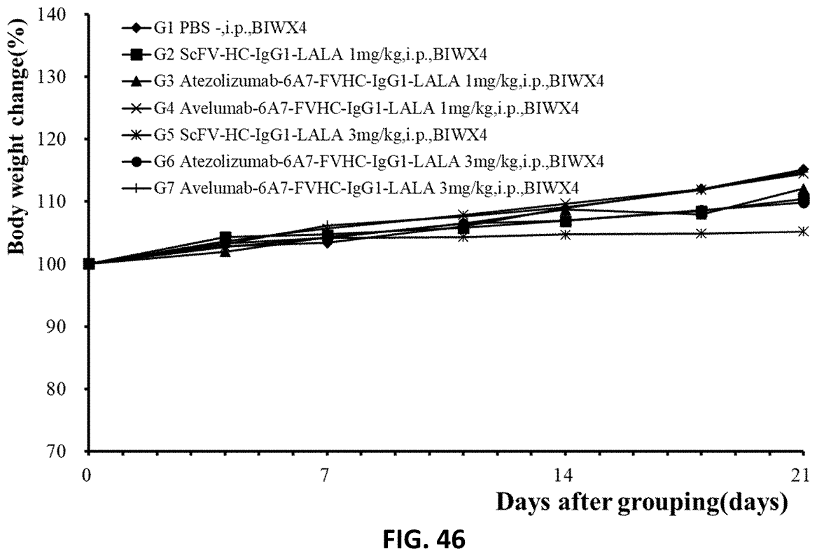

The weight of the mice was monitored during the entire treatment period. The average weight of mice in different groups all increased to different extents (FIG. 45, and FIG. 46). All the mice gained weight among different groups at the end of the treatment period.

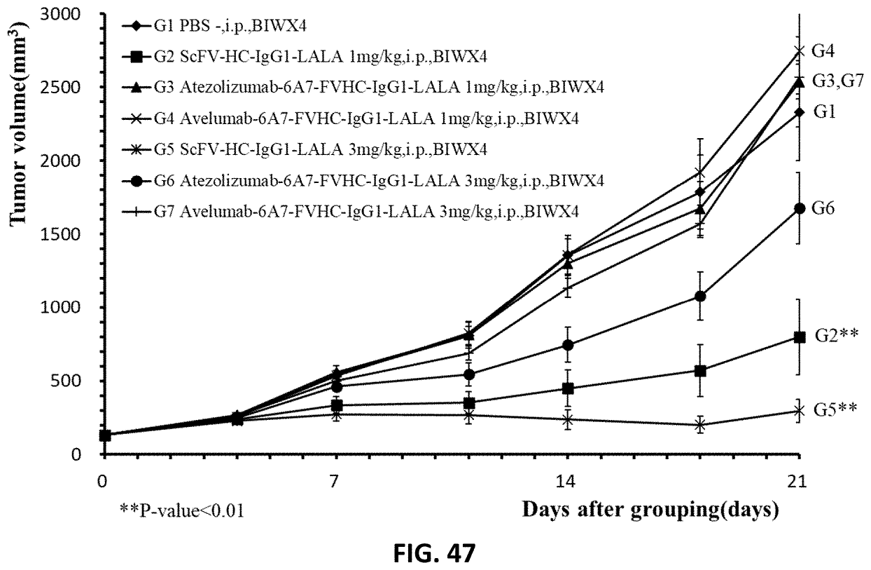

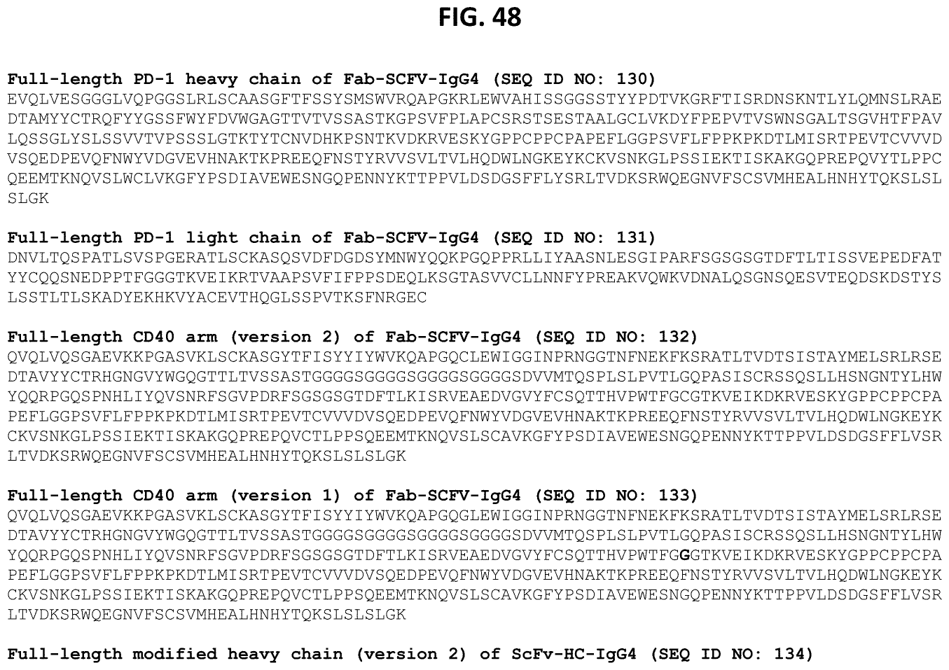

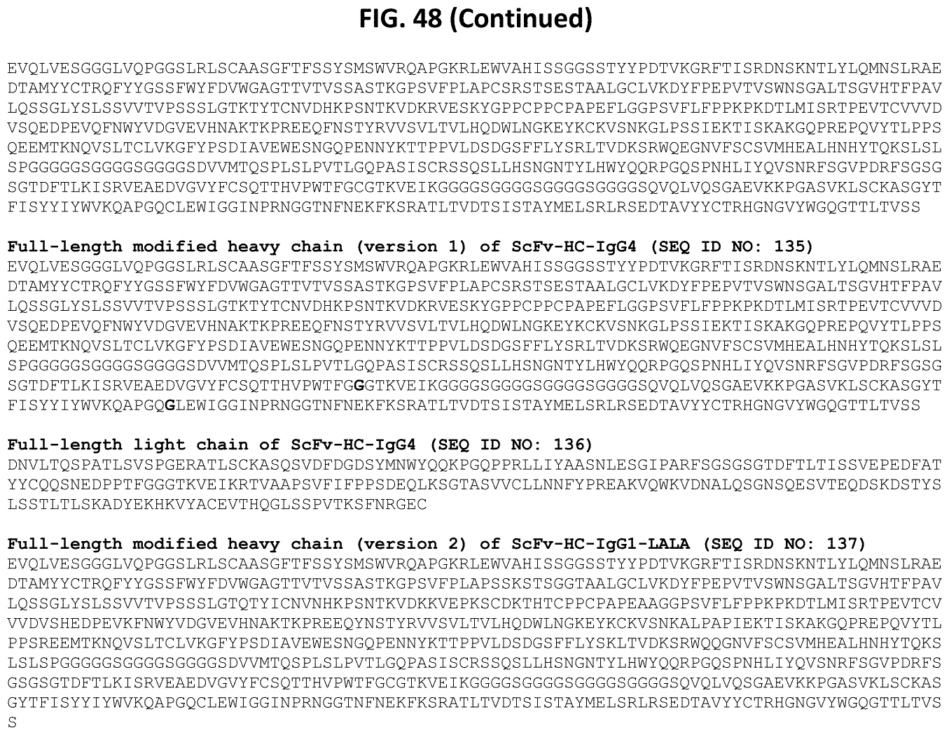

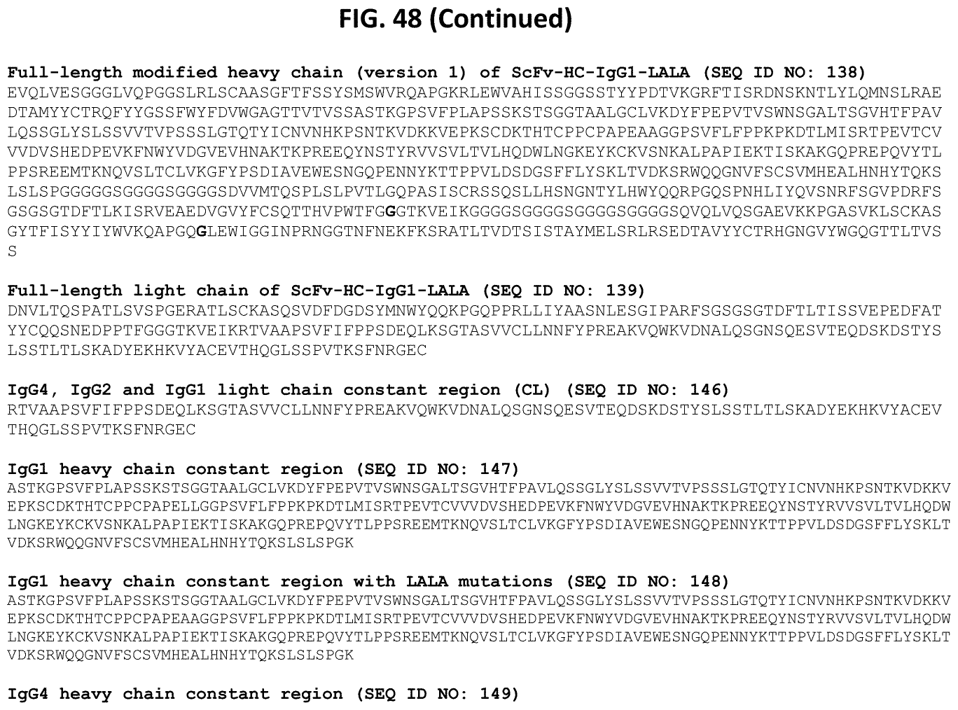

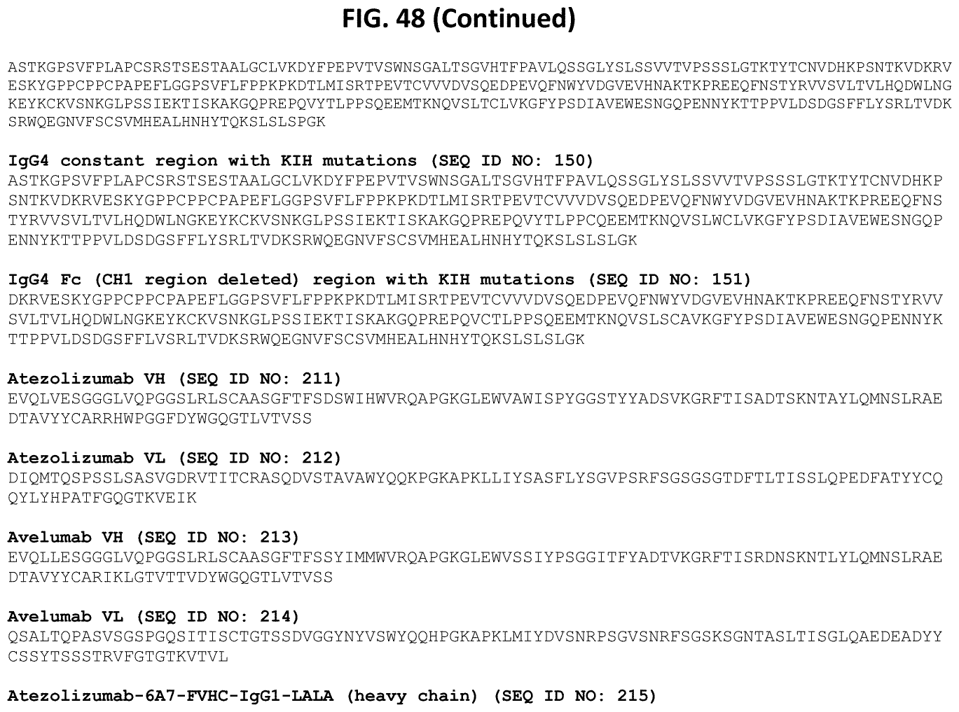

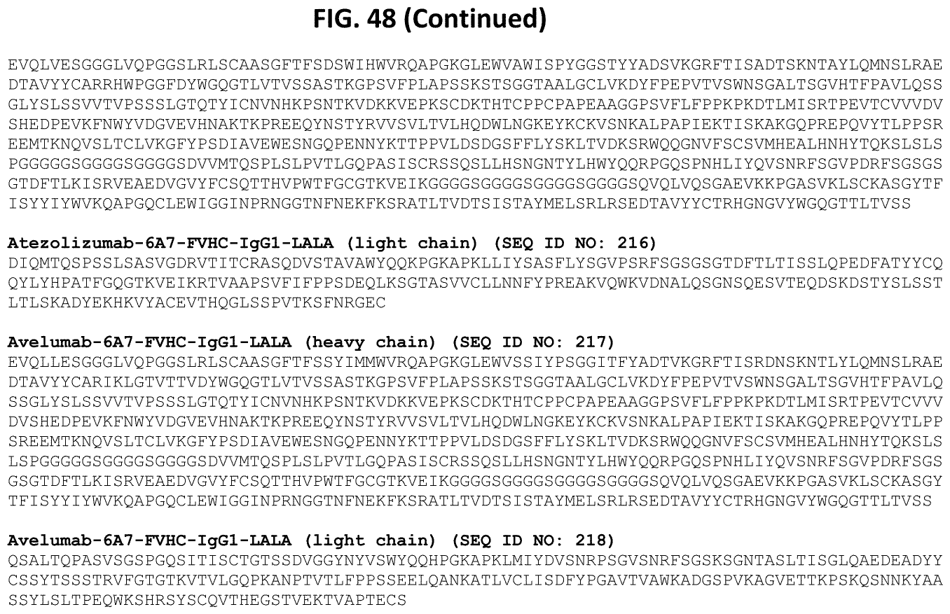

The tumor size in groups treated with the antibodies is shown in FIG. 47. The TGI_{TV}% on Day 21 (21 days after grouping) was calculated as shown in the table below. P values in the following table was calculated based on the data on Day 21.

TABLE 33

| | | | | | | | P value for | |
|---|---|---|---|---|---|---|---|---|
| | | Tumor volume (mm³) | | | Survival | TGI_{TV} | Day 21 | |
| | | Day | Day | Day | on Day | % on Day | Body | Tumor |
| | | 0 | 14 | 21 | 21 | 21 | weight | Volume |
| Control | G1 | 131 ± 3 | 1354 ± 137 | 2328 ± 328 | 6/6 | n.a. | n.a. | n.a. |
| Treat | G2 | 131 ± 3 | 450 ± 125 | 799 ± 257 | 6/6 | 69.6% | 0.036 | 0.004 |
| | G3 | 131 ± 4 | 1300 ± 166 | 2536 ± 306 | 6/6 | −9.5% | 0.419 | 0.635 |
| | G4 | 131 ± 4 | 1359 ± 133 | 2747 ± 326 | 6/6 | −19.1% | 1.000 | 0.386 |
| | G5 | 131 ± 4 | 237 ± 66 | 296 ± 77 | 6/6 | 92.5% | 0.005 | 1.28E−04 |
| | G6 | 131 ± 5 | 745 ± 119 | 1676 ± 245 | 6/6 | 29.7% | 0.033 | 0.142 |
| | G7 | 131 ± 6 | 1135 ± 64 | 2569 ± 115 | 6/6 | −11.0% | 0.663 | 0.504 |

The results showed that the ScFV-HC-IgG1-LALA antibody showed significantly better in vivo efficacy than Atezolizumab-6A7-FVHC-IgG1-LALA or Avelumab-6A7-FVHC-IgG1-LALA at the same dose level. The higher the dose level, the more effective the treatment was. More specifically, the results showed that ScFV-HC-IgG1(G2, G5) inhibited tumor growth with a higher TGI_{TV}% (69.6%, 92.5%) than that of the antibodies Atezolizumab-6A7-FVHC-IgG1-LALA (G3, G6) or Avelumab-6A7-FVHC-IgG1-LALA (G4, G7) at different dose levels, and higher doses led to better therapeutic effects. Therefore, it demonstrated that different molecules having the ScFV-HC-IgG1 format can have different therapeutic effects inside B-hPD-1/hPD-L1/hCD40 mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having cancer, the method comprising administering a therapeutically effective amount of a composition comprising an antigen-binding protein construct to the subject, wherein the antigen-binding protein construct comprises a first antigen-binding site that specifically binds to PD-1, and a second antigen-binding site that specifically binds to CD40.

2. The method of claim 1, wherein the antigen-binding protein construct is capable of activating CD40 pathway, wherein the activation of CD40 pathway depends on the binding of the antigen-binding protein construct to a cell expressing PD-1.

3. The method of claim 1, wherein the antigen-binding protein construct induces CD40 pathway activities in the presence of one or more cells expressing PD-1.

4. The method of claim 1, wherein the antigen-binding protein construct induces CD40 pathway activities in a tumor microenvironment or a tumor-draining lymph node.

5. The method of claim 1, wherein the antigen-binding protein construct comprises an Fc region, wherein the second antigen-binding site is linked to the Fc region.

6. The method of claim 1, wherein the antigen-binding protein construct comprises an Fc region, wherein the second antigen-binding site is linked to the C-terminal of the Fc region.

7. The method of claim 1, wherein the antigen-binding protein construct comprises an Fc region, wherein the antigen-binding protein construct is incapable of activating CD40 pathway through Fc receptor-mediated activity.

8. The method of claim 1, wherein the first antigen-binding site that specifically binds to PD-1 comprises a ScFv, a VHH domain, or a PD-1 ligand or a soluble portion thereof

9. The method of claim 1, wherein the second antigen-binding site that specifically binds to CD40 comprises a ScFv, a VHH domain, or a CD40 ligand or a soluble portion thereof

10. An antigen-binding protein construct, comprising
a first heavy chain variable region and a first light chain variable region, wherein the first heavy chain variable region and the first light chain variable region associate with each other, forming a first antigen binding site that specifically binds to PD-1; and
a second heavy chain variable region and a second light chain variable region, wherein the second heavy chain variable region and the second light chain variable region associate with each other, forming a second antigen binding site that specifically binds to CD40.

11. The antigen-binding protein construct of claim 10, wherein the antigen-binding protein construct comprises
a first polypeptide comprising the first heavy chain variable region, a first heavy chain constant region 2 (CH2), and a first heavy chain constant region 3 (CH3); and
a second polypeptide comprising the second heavy chain variable region, a second heavy chain constant region 2 (CH2), and a second heavy chain constant region 3 (CH3).

12. The antigen-binding protein construct of claim 11, wherein the second polypeptide further comprises the second light chain variable region.

13. The antigen-binding protein construct of claim 12, wherein the antigen-binding protein construct comprises a third polypeptide comprising the first light chain variable region.

14. The antigen-binding protein construct of claim 10, wherein the antigen-binding protein construct comprises
a first polypeptide comprising the first heavy chain variable region, the second heavy chain variable region, and the second light chain variable region; and
a second polypeptide comprising the first light chain variable region.

15. The antigen-binding protein construct of claim 14, wherein the first polypeptide further comprises a heavy chain constant region 1 (CH1), a heavy chain constant region 2 (CH2), and a heavy chain constant region 3 (CH3).

16. The method of claim 1, wherein the antigen-binding protein construct is a TrioMab, a bispecific antibody with a common light chain, a CrossMab, a 2:1 CrossMab, a 2:2 CrossMab, a Duobody, a Dual-variable-domain antibody (DVD-Ig), a scFv-IgG, a IgG-IgG format antibody, a Fab-scFv-Fc format antibody, a TF, an ADAPTIR, a Bispecific T cell Engager (BiTE), a BiTE-Fc, a Dual affinity retargeting (DART), a DART-Fc, a tetravalent DART, a Tandem diabody (TandAb), a scFv-scFv-scFv, an ImmTAC, a Tri-specific nanobody, or a Trispecific Killer Engager (TriKE).

17. An antigen-binding protein construct, comprising a first antigen-binding site that specifically binds to PD-1, and a second antigen-binding site that specifically binds to CD40.

18. The antigen-binding protein construct of claim 17, comprising
a heavy chain polypeptide comprising a first heavy chain variable region;
a light chain polypeptide comprising a first light chain variable region; and
a single-chain variable fragment polypeptide comprising a second heavy chain variable region, and a second light chain variable region,
wherein the first heavy chain variable region and the first light chain variable region associate with each other, forming the first antigen binding site that specifically binds to PD-1, and the second heavy chain variable region and the second light chain variable region associate with each other, forming the second antigen binding site that specifically binds to CD40.

19. The antigen-binding protein construct of claim 17, comprising
a heavy chain polypeptide comprising a first heavy chain variable region, and
a light chain polypeptide comprising a first light chain variable region;
wherein a single-chain variable fragment polypeptide is linked to the C-terminus of the heavy chain polypeptide; wherein the single-chain variable fragment polypeptide comprises a second heavy chain variable region and a second light chain variable region;
wherein the first heavy chain variable region and the first light chain variable region associate with each other, forming the first antigen binding site that specifically binds to PD-1, and the second heavy chain variable region and the second light chain variable region associate with each other, forming the second antigen binding site that specifically binds to CD40.

20. A method of decreasing the rate of tumor growth, the method comprising administering to a subject in need thereof an effective amount of a composition comprising the antigen-binding protein construct of claim 17, to the subject.

21. A method of killing a tumor cell, the method comprising contacting the tumor cell with an effective amount of a composition comprising the antigen-binding protein construct of claim 17.

22. A pharmaceutical composition comprising the antigen-binding protein construct of claim 17, and a pharmaceutically acceptable carrier.
